(19) United States
(12) Patent Application Publication
(10) Pub. No.: US 2025/0170221 A1
(43) Pub. Date: May 29, 2025

(54) USE OF LENTIVECTOR-TRANSDUCED T-RAPA CELLS FOR AMELIORATION OF LYSOSOMAL STORAGE DISORDERS

(71) Applicant: The Medical College of Wisconsin, Inc., Milwaukee, WI (US)

(72) Inventors: Jeffrey A. Medin, Shorewood, WI (US); Daniel H. Fowler, Milwaukee, WI (US); Murtaza S. Nagree, Milwaukee, WI (US); Tania Felizardo, Milwaukee, WI (US)

(21) Appl. No.: 19/041,531

(22) Filed: Jan. 30, 2025

Related U.S. Application Data
(62) Division of application No. 17/050,949, filed on Oct. 27, 2020, now Pat. No. 12,239,693.
(60) Provisional application No. 62/663,786, filed on Apr. 27, 2018.

(51) Int. Cl. A61K 38/44; A61K 38/47; A61K 40/11; A61K 40/40; A61P 43/00; C12N 5/0783; C12N 15/79; C12N 15/86

(52) U.S. Cl. CPC A61K 38/443; A61K 38/47; A61K 40/11; A61K 40/40; A61P 43/00; C12N 5/0636; C12N 15/86; A61K 2239/31; A61K 2239/38; C12N 15/79; C12N 2510/00; C12Y 101/01205; C12Y 302/01022

(57) ABSTRACT

The present disclosure provides methods of treating lysosomal storage disorders, e.g., Fabry disease, Gaucher disease, Farber disease, and Pompe disease. The method comprises producing vector-transduced T-Rapa cells that express a transgene of interest and administering the cells to a patient in need thereof. The T-Rapa cells may be transduced with a dual promoter lentivirus vector.

### CROSS-REFERENCE TO RELATED APPLICATIONS

This application is a divisional of U.S. patent application Ser. No. 17/050,949, filed Oct. 27, 2020, which is the U.S. National Stage entry of PCT/US2019/029639, filed Apr. 29, 2019, which claims priority to U.S. Provisional Application No. 62/663,786, filed on Apr. 27, 2018, the contents of each of which are incorporated by reference in their entireties.

### STATEMENT REGARDING FEDERALLY SPONSORED RESEARCH

This invention was made with government support under Project Number ZIA BC 011219 awarded by the National Cancer Institute. The government has certain rights in the invention.

### REFERENCE TO A SEQUENCE LISTING

A Sequence Listing accompanies this application and is submitted as an xml file of the sequence listing named “650053_01130.xml” which is 88,477 bytes in size and was created on Jan. 28, 2025. The sequence listing is electronically submitted via Patent Center and is incorporated by reference herein in its entirety.

### BACKGROUND OF THE INVENTION

The field of the invention is treatment of lysosomal storage disorders (LSD, lysosomal storage diseases). LSD are associated with dysregulation or deficiency of a single protein (such as Fabry disease) or combinations of an enzyme deficiency and co-activator proteins.

Lysosomes are membrane-bound organelles in eukaryotic cells that contain more than 60 different enzymes capable of digesting nearly any biomolecule. They carry out many critical biological functions, including acting as the cell's waste disposal system by digesting unwanted materials in the cytoplasm, both from outside of the cell and obsolete components inside the cell. Lysosomal Storage Disorders (LSD) are a group of more than 60 rare inherited metabolic disorders that result from lysosome dysfunction, usually as a consequence of a deficiency in a single enzyme required for the intracellular digestion of lipids, glycoproteins or polysaccharides. As a result of such deficiencies, the molecules that would normally be degraded accumulate inside the cell, leading to dysfunction or death of the cell.

Fabry disease is a LSD resulting from a deficiency in the enzyme 60 -galactosidase A (α-gal A encoded by the AGA transgene), an enzyme that hydrolyses α-galactose from glycosphingolipids, in particular globotriaosylceramide (Gb₃).

The standard-of-care treatment for Fabry disease is enzyme replacement therapy (ERT). The efficacy of ERT is outlined by Rombach et al. (Orphanet J Rare Dis. 8:47-10.1186/1750-1172-8-47 (2013)). While some benefits can be obtained, disease progression is not halted. ERT requires lengthy intravenous infusions of recombinant α-gal A administered every couple of weeks, often at an outpatient center. Fabry patients often require treatment for pain, gastrointestinal dysfunction, arrhythmias and other heart problems, as well as needing blood thinners and blood pressure medications to protect kidney functions. Although Fabry disease is relatively rare, there are about 4000 patients in the US, treatment costs are on the order of $300,000/year/patient ($1.2 B/year for all US patients).

Hematopoietic stem cells (HSCs) are “multipotent” cells residing in bone marrow that can ultimately differentiate into all blood cell types. A characteristic of HSCs is their expression of a cell surface glycoprotein called CD34, and such cells are sometimes referred to as CD34+ hematopoietic cells, or more simply as CD34+ HSCs. Clinically, the presence of CD34 on HSCs can be used to facilitate selective enrichment of HSCs for bone marrow transplants. In addition, CD34+ HSCs have been used experimentally to treat a variety of non-hematopoietic diseases including spinal cord injuries, liver cirrhosis, and peripheral vascular disease. HSCs can be harvested from bone marrow, but may also be harvested from peripheral blood after treatment with certain drugs to ‘mobilize’ them. Thus, HSCs can be harvested from blood (e.g., by apheresis). HSCs are also “mobile”, meaning that they can move from bone marrow into the blood stream to different sites in the body. HSCs can be administered by injection into the blood stream in order to repopulate bone marrow.

The inventors have previously used HSCs harvested from Fabry patients to genetically modify the HSCs to produce α-gal A, the enzyme deficient in patients with Fabry disease. These genetically modified HSCs are infused back into the same patients (autologous grafts) after patients have been “conditioned” by drug regimens to ablate the endogenous HSCs in order to improve the success of the therapy.

Upon re-introduction of the patient's modified cells back into the patient, the genetically modified HSCs will populate all downstream lineages of the hematopoietic system and then circulate throughout the body. The modified cells secrete a form of α-gal A with a molecular “tag” (mannose-6-phosphate) which enables uncorrected “bystander” cells in the patient to take up and transport the α-gal A intracellularly into their lysosomes, where they compensate for the patient's α-gal A deficiency, and effectively degrade the accumulated glycosphingolipids. This method is undergoing clinical trials in Canada (ClinicalTrials.gov #NCT02800070).

The core tenet of the prior protocol is that the genetically modified HSCs will differentiate into all possible blood cell (hematopoietic) lineages and circulate throughout the body. However, due to inefficiency of engraftment, it is necessary to condition recipients by hematologic ablation. The degree of ablation can determine the efficiency of engraftment in most cases. In addition, there is a limitation on the numbers of transduced bonafide stem cells that can be obtained and employed to correct the disease. Even using autologous grafts, additional rounds of transplantation may be necessary to effectively treat the disease.

Thus, there is a need to fine a renewable source of cells within a subject that can be used for therapy and will require minimal or no ablation of the patient.

### SUMMARY OF THE INVENTION

The present invention overcomes the aforementioned drawbacks by providing methods of treating lysosomal storage disorders.

In one aspect, the disclosure provides a method of treating a lysosomal storage disorder in a subject, the method comprising the steps of: (a) conditioning T-cells from the subject or suitable donor with rapamycin ex vivo to generate T-Rapa cells; (b) transducing the T-cells in vitro with a vector comprising a transgene of interest that encodes an enzyme associated with a lysosomal storage disorder; and (c) administering the transduced T-Rapa cells to the subject, wherein the T-Rapa cells express the enzyme associated with the lysosomal storage disorder in the subject and reduce one or more symptoms of the lysosomal storage disorder. In some aspect, the method after step (b) comprises expanding the vector-transduced T-Rapa cells by culturing in vitro, and step (c) comprises administering the transduced and expanded T-Rapa cells to the subject.

In another aspect, the disclosure provides a method of treating a lysosomal storage disorder in a subject, the method comprising the steps of: (a) obtaining T-cells from the subject or a suitable donor, (b) conditioning the T-cells with rapamycin ex vivo to generate T-Rapa cells; (c) transducing the T-cells in vitro with a vector that expresses the transgene of interest in the T-Rapa cells; (d) in vitro expanding the vector-transduced T-Rapa cells in culture, and (e) administering T-Rapa cells into the subject, wherein the T-Rapa cells express the transgene of interest in the subject and reduce one or more symptoms of the lysosomal storage disorder.

In some embodiments, the administering step is by transfusion or intravenous injection.

In some aspects the method further comprises maintaining and expanding the vector-transduced T-Rapa cells in in vitro culture and storing a portion of the vector-transduced T-Rapa cells for future administration to the subject.

In another aspect, the disclosure provides a method of producing a population of transduced T-Rapa cells that express an enzyme encoded by a transgene of interest for the treatment of a lysosomal storage disorder, the method comprising: (a) conditioning T-cells from a subject or a suitable donor with rapamycin, producing a population of T-Rapa cells; and (b) transducing the T-Rapa cells in vitro with a vector comprising the transgene of interest to produce a population of transduced T-Rapa cells. This method produces transduced T-Rapa cells able to express the protein (e.g. enzyme) encoded by the transgene of interest. In some aspects, the method further comprises (c) in vitro expanding the vector-transduced T-Rapa cells in culture.

In another aspect, the disclosure provides a method of producing a population of transduced T-Rapa cells that express a transgene of interest for the treatment of a lysosomal storage disorder, the method comprising: (a) obtaining T-cells from the subject or a suitable donor, (b) conditioning the T-cells with rapamycin, producing T-Rapa cells; and (c) transducing the T-Rapa cells in vitro with a vector that expresses a transgene of interest in the T-Rapa cell. In some aspects, the method further comprises (d) in vitro expanding the vector-transduced T-Rapa cells in culture.

In yet another aspect, the disclosure provides a method of treating a subject with Fabry disease, the method comprising administering an effective amount of the transduced T-Rapa cells made by the method described herein that express α-gal A to treat one or more symptoms of Fabry disease.

In another aspect, the disclosure provides a population of transduced T-Rapa cells that express a protein encoded by a transgene of interest. In one aspect, the disclosure provides a population of transduced T-Rapa cells that express α-gal A. In another aspect, the disclosure provides a population of transduced T-Rapa cells that express β-glucocerebrosidase. In another aspect, the disclosure provides a population of transduced T-Rapa cells that express acid ceramidase. In another aspect, the disclosure provides a population of transduce T-Rapa cells that express acid α-glucosidase.

In yet another aspect, the disclosure provides a method of treating a subject with Gaucher disease, the method comprising administering an effective amount of the transduced T-Rapa cells that express GBA to treat one or more symptoms of Gaucher disease.

In yet another aspect, the disclosure provides a method of treating a subject with Farber disease, the method comprising administering an effective amount of the transduced T-Rapa cells that express ASAH1 to treat one or more symptoms of Farber disease.

In a further aspect, the disclosure provides a method of treating a subject with Pompe disease, the method comprising administering an effective amount of the transduced T-Rapa cells that express GAA to treat one or more symptoms of Pompe disease.

In yet another aspect, the disclosure provides a method of treating a subject with a lysosomal storage disorder, the method comprising administering transduced T-Rapa cells expressing a transgene associated with treatment of the lysosomal storage disorder in an effective amount to treat one or more symptom of the lysosomal storage disorder.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of prior methods of hematopoietic stem cell gene therapy.

FIG. 2 are graphs depicting the transduction efficiency of T-Rapa cells isolated from healthy and Fabry donor using vehicle only (NT) or α-gal A-expressing lentiviral vector (LV/AGA).

FIGS. 3A-3B depict the a-gal A expression in transduced T-Rapa cells from healthy and Fabry donors. Expression of α-gal A was determined both in the supernatant (B) or the cell lysates (A) of the transduced cells.

FIG. 4 is a representative western blot of α-gal A proteins level in normal donor- and Fabry Donor-derived transduced T-Rapa cells performed by Wes™ Western Blot System.

FIG. 5 is the quantitation of α-gal A in normal donor and Fabry donor transduced T-Rapa cells quantitated by Wes™ Western Blot System.

FIG. 6 is a schematic of the protocol for in vivo testing of the transduced T-Rapa cells in the NOD/SCID/Aga^{-/-} Fabry mouse model.

FIG. 7 are graphs depicting the enzyme activity levels of α-gal A in the plasma of mice engrafted with normal donor- or Fabry donor-derived transduced T-Rapa cells.

FIG. 8 is the raw data from the mouse experiments of Example 5 and FIG. 7.

FIG. 9 is a schematic representing the method of producing transduced T-Rapa cells.

FIG. 10 is an exemplary schematic representing the method of treating a patient with Fabry disease according to the current invention. Alternative viruses used are contemplated and this is only exemplary.

FIG. 11 is a cartoon depiction of one suitable method of making lentiviral vectors for use in the present methods.

FIG. 12A is a schematic of a suitable lentiviral vector able to be used in the present methods; pDY/CO.α-galA (i.e., LV/AGA) to express α-gal A. LTR, long terminal repeat, Ψ, HIV packaging signal; SD, 5′ splice donor site; ΔGAG, truncated portion of HIV-1 group specific antigen gene; RRE, Rev-response element; SA, 3′ splice acceptor site; cPPT, central polypurine tract; EF-1α, elongation factor 1 a promoter; CO.α-gal A, codon-optimized cDNA of the human GLA gene encoding the wild-type α-gal A enzyme; WPRE, woodchuck hepatitis virus post-transcriptional regulatory element; SIN/LTR, self-inactivating LTR.

FIG. 12B is a schematic of a dual promoter lentiviral vector containing codon optimized and mutated IMPDH2 cDNA sequence (IMPDH2(IY)) and AGA codon optimized transgene as described in FIG. 12A.

FIG. 12C shows exemplary schema of lentiviral vectors containing the GBA, ASAH1 and GAA gene, respectively.

FIG. 13A is the plasmid map of the lentiviral vector expressing α-gal A (SEQ ID NO:3).

FIG. 13B is a plasmid map of dual promoter lentiviral vector containing the mutant IMPDH2 gene (IMPDH2(IY)) and AGA codon optimized gene (vector: SEQ ID NO:4).

FIG. 13C is a plasmid map made of the lentiviral vector containing a wild-type codon optimized GBA transgene (SEQ ID NO:4).

FIG. 13D is a plasmid map of the lentiviral vector containing a wild-type ASAH1 transgene (SEQ ID NO5).

FIG. 13E is a plasmid map of the lentiviral vector containing a wild-type codon optimized GAA transgene (SEQ ID NO:6).

FIG. 14 demonstrates the ability of the lentiviruses encoding AGA, GBA, ASAH1 or GAA to express and produce the corresponding enzyme within transduced HEK293T cells.

FIG. 15 demonstrates the ability of the lentiviruses encoding the enzymes of the transgenes AGA, GBA, ASAH1 or GAA to express and secrete the corresponding enzyme within transduced Jurkat cells.

FIG. 16 demonstrates the ability of the lentivirus vectors encoding the enzyme associated with either AGA transgene or GBA transgene to be transduced into T-Rapa cells (from healthy donors), and the ability of the T-Rapa cells to survive cryopreservation and subsequent thawing.

FIG. 17 demonstrates transduced T-Rapa cells derived from Fabry patients are able to produce α-gal A after transduction (left panel) and that α-gal A produced from Fabry patient transduced T-Rapa cells can be taken up by patient cells due to their molecular ‘tag’ (right panel).

FIG. 18 demonstrates the ability of human transduced T-Rapa cells to produce enzyme in vivo when transplanted into immunocompromised Fabry mice after conditioning. Engraftment was confirmed by detecting hCD3/hCD4 in peripheral blood. α-gal A activity was detectable in vivo 4 weeks after xenograft of transduced healthy donor T-Rapa (n=4-5) in plasma or lysates of the indicated tissue (liver, spleen, heart or kidney).

FIG. 19 demonstrates the ability of the lentiviral transduced human T-Rapa cells to reduce substrate globotriaosylceramide (Gb₃) (the primary substrate that accumulates in Fabry mice) after transplant into immunocompromised Fabry mice.

FIG. 20 demonstrates the ability of the Fabry patient T-Rapa cells transduced with the lentivirus encoding AGA to be able to secrete α-gal A in vivo. α-gal A activity is detectable in vivo 4 weeks after xenograft of transduced Fabry donor T Rapa (n=4-5) in the indicated tissues.

FIG. 21 demonstrates the ability of the lentiviral transduced Fabry patient T-Rapa cells to reduce substrate globotriaosylceramide (Gb₃) (the primary substrate that accumulates in Fabry mice) after transplant into immunocompromised Fabry mice.

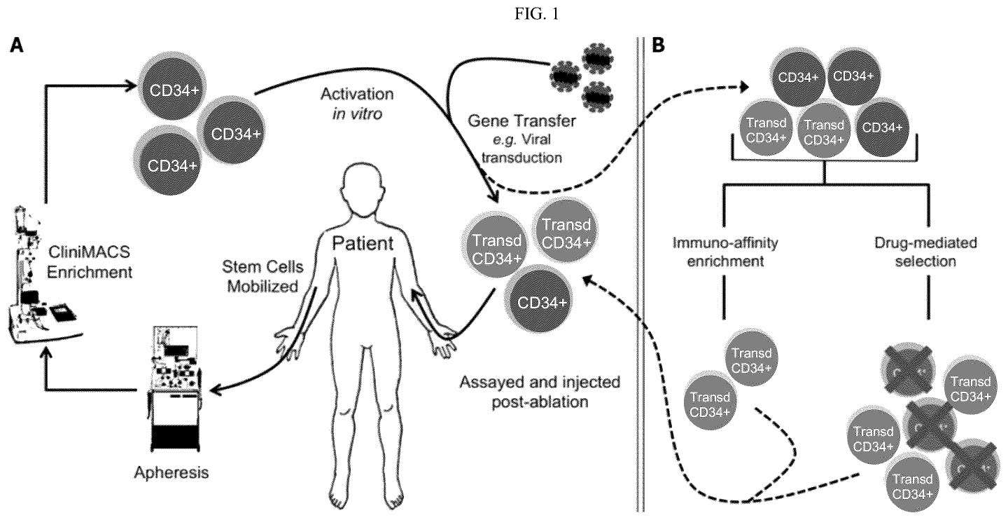

### DETAILED DESCRIPTION OF THE INVENTION

Prior methods of using hematopoietic stem cell (HSC)-directed gene therapy are being tested in amenable lysosomal storage deficiencies which are caused by a single enzyme deficiency. As an example, the inventors are currently conducting a phase I clinical trial (NCT02800070) aimed at treating patients with Fabry disease (FD) by gene transfer. FD is an a-galactosidase A (α-gal A) deficiency in which globotriaosylceramide (Gb₃) and other metabolites accumulate. In this prior protocol, CD34+ hematopoietic cells are transduced ex vivo with a recombinant lentivirus (LV) engineered to overexpress α-gal A. These cells are then returned to the patient. Cells derived from the vector-transduced HSCs, including leukocytes, can secrete α-gal A and uncorrected cells within the patient can take up the secreted α-gal A, a process termed “cross-correction”. For efficient engraftment, patients receive conditioning regimens (e.g., ablation) that can be problematic. In addition, HSCs must be mobilized to peripheral blood with drugs and collected via apheresis. Alternative circulating cell populations that are easier to obtain and transplant are more desirable to use to deliver therapeutic cargo systemically.

The present disclosure provides improved methods of treating a lysosomal storage disorders, particularly Fabry disease (Online Mendelian Inheritance in Man (OMIM) ID #301500, Gaucher disease (OMIM ID #230800, 230900, 231000, 231005), Farber disease (OMIM ID #228000), and Pompe disease (OMIM ID #232300)). Particularly, the present invention describes the use of autologous or donor (non-autologous) CD4+ T-Rapa cells to deliver therapeutic transgene products systemically. T-Rapa cells can be manufactured from peripheral blood cells of affected patients or normal donors (ND) and can be productively transduced with a vector (e.g., lentiviral vector) comprising a transgene (e.g. sequence encoding an enzyme) lacking in the disease, for example, but not limited to, the enzymes α-gal A for Fabry disease, beta-glucocerebrosidase (GBA, β-Glucocerebrosidase, acid β-glucosidase, D-glucosyl-N-acylsphingosine glucohydrolase, or GCase, which can be used interchangeably) for Gaucher disease, acid ceramidase (encoded by the ASAH1 transgene) for Farber disease, and acid α-glucosidase (encoded by GAA transgene, also known as acid maltase) for Pompe disease. The present disclosure provides an improved method that uses cells obtained from the peripheral blood (e.g. T-cells) and can provide a population of transduced T-Rapa cells expressing the enzyme that can be stored and infused at any time to boost in vivo circulating transgene-producing T-Rapa cells when needed. Further, the method requires low, if any, ablation to provide efficient engraftment into the subject.

T-cells are natural protein-secreting machines and are already employed in many clinical trials. Unlike HSCs, T-cells can be obtained from peripheral blood (PB) without mobilization and can be expanded exponentially in culture. Ex vivo treatment with rapamycin elicits numerous changes in T-cells (e.g., CD4+ T-cells) that, in sum, endow them with a pro-engraftment and anti-apoptotic phenotype. These are termed T-Rapa cells. More about T-Rapa cells can be found in Fowler et al. (“Phase 2 clinical trial of rapamycin-resistant donor CD4+ Th2/Th1 (T-Rapa) cells after low-intensity allogeneic hematopoietic cell transplantation,” Blood (2013) 11:121 (15):2864-2874), the contents of which are incorporated by reference in its entirety. Successful allotransplantation of donor T-Rapa cells requires less host conditioning (lymphocyte-specific, myeloid sparing) that results in the creation of sufficient immune space for T-cell engraftment while causing minimal host myeloid cell depletion. This method of host conditioning and T-cell-driven gene therapy is substantially different from HSC-driven gene therapy, which typically requires relatively intense myeloid cell depletion. Not to be bound by any theory, but it is advantageous to administer gene therapy via T-cells rather than HSC from several perspectives, including the fact that reduction in myeloid cell depletion: will reduce infectious complications that are associated with myeloid depletion; allow gene therapy to be performed in the outpatient setting, which will lower treatment morbidity and cost; and will allow repetitive dosing of gene therapy, which will ultimately improve efficacy.

As demonstrated in the Examples, after in vitro expansion for 2 weeks, transduced T-Rapa cells continue to secrete the transgene-product (e.g. enzyme, such as α-gal A) in the absence of stimulation in vitro. Transduced and control T-Rapa cells from FD patients and normal donors were xenografted into NOD/SCID/Aga^{-/-} mice (NSF). Higher α-gal A activity was detected in plasma and organs of mice given LV-modified cells. Vector copy number analyses suggest stable transduction. NSF mice receiving transduced cells also exhibited reduced Gb₃ levels, demonstrating the ability of the enzyme being expressed from the transduced T-Rapa cells to reduce the in vivo substrate target.

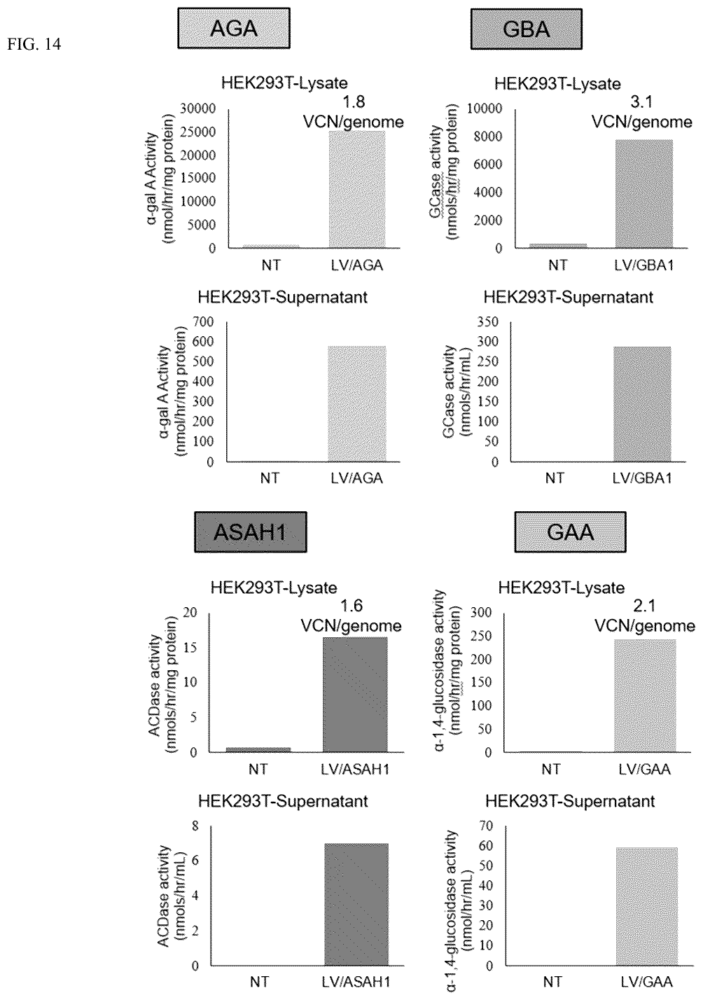

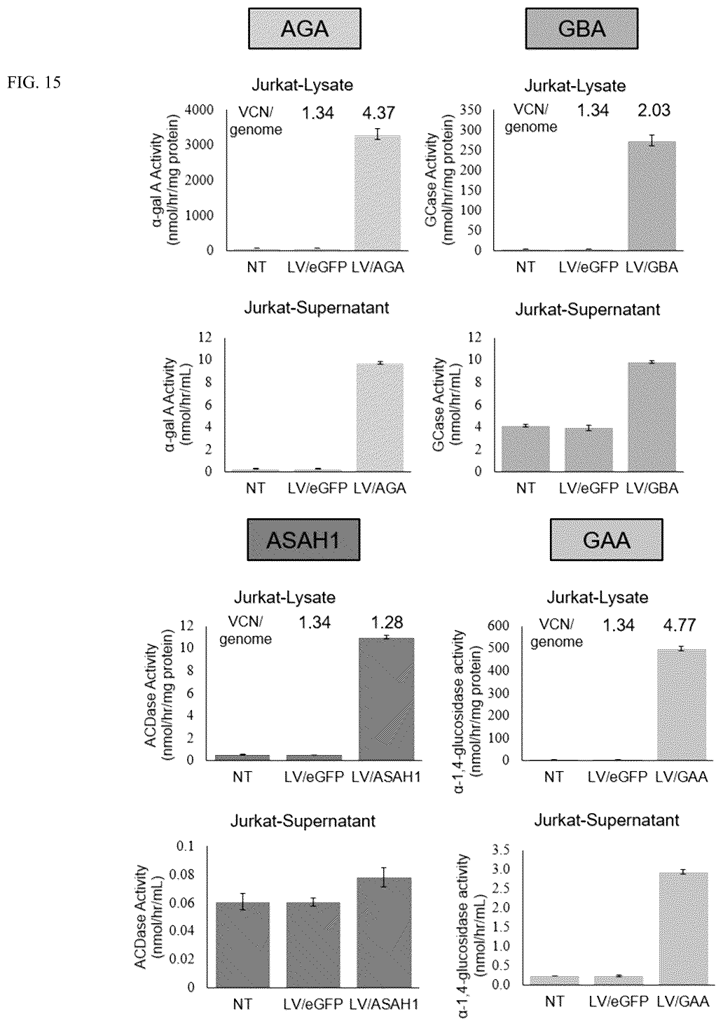

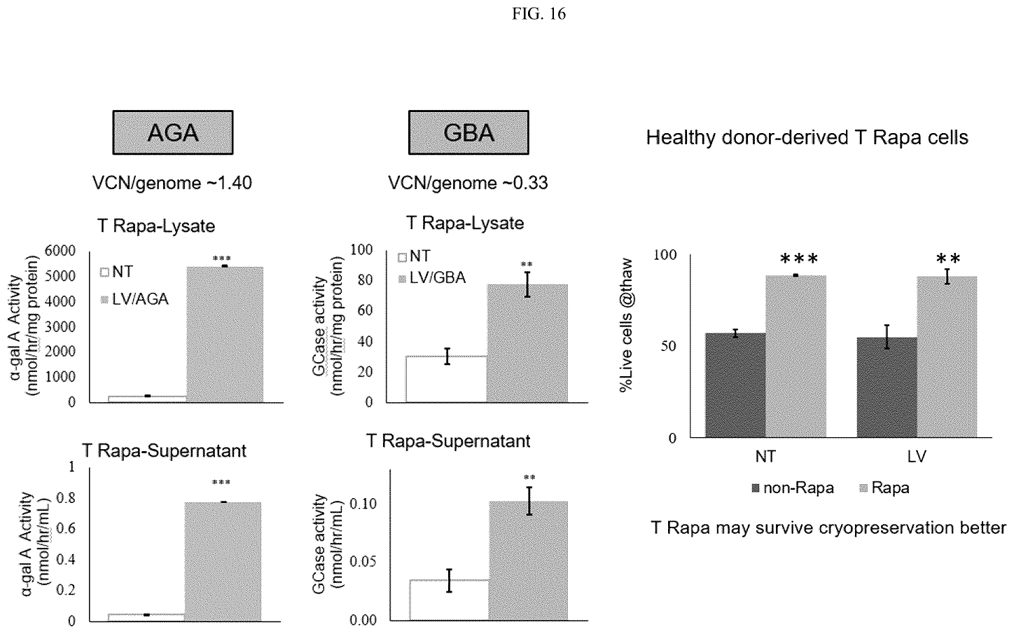

The Examples demonstrate the in vitro development of lentiviral-transduced T-Rapa cells that can lead to increased enzyme activity and secretion of enzymes from cells. While the Examples demonstrate the use of lentiviral-transduced T-Rapa cells that increase α-gal A activity which can be used to treat Fabry disease, lentiviral-transduced T-Rapa cells can be used for expressing other enzymes to treat other lysosomal storage disorders, as shown in FIGS. 14 and 15 for the transgenes GBA, ASAH1 and GAA.

In one embodiment, the disclosure provides a method of treating a lysosomal storage disorder in a subject, the method comprising the steps of: (a) conditioning T-cells from the subject or suitable donor with rapamycin ex vivo to generate T-Rapa cells; (b) transducing the T-cells in vitro with a vector comprising a transgene of interest that encodes an enzyme associated with a lysosomal storage disorder; and (c) administering the transduced T-Rapa cells to the subject, wherein the T-Rapa cells express the enzyme associated with a lysosomal storage disorder in the subject and reduce one or more symptoms of the lysosomal storage disorder. In some embodiment, the method after step (b) comprises expanding the vector-transduced T-Rapa cells by culturing in vitro before administering the transduced and expanded T-Rapa cells in step (c).

Suitable methods of administering the transduced T-Rapa cells are known in the art, and include, transfusion and intravenous administration.

In one embodiment, the disclosure provides a method of treating a lysosomal storage disorder in a subject. The method comprising the steps of: (a) conditioning T-cells with an effective amount of rapamycin ex vivo to produce T-Rapa cells; (b) transducing the T-Rapa cells in vitro with a vector that comprises the transgene of interest that encodes the enzyme associated with the lysosomal storage disease; (c) expanding the vector-transduced T-Rapa cells in in vitro culture, and (d) administering the transduced T-Rapa cells into the subject, wherein the T-Rapa cells express the protein encoded by the transgene of interest in the subject and can subsequently reduce one or more symptoms of the lysosomal storage disorder.

In another embodiment, the disclosure provides a method of treating a lysosomal storage disorder in a subject. The method comprising the steps of: (a) obtaining T-cells from the subject or a suitable donor, (b) conditioning the T-cells with rapamycin ex vivo to produce T-Rapa cells; (c) transducing the T-Rapa cells in vitro with a vector that expresses the transgene of interest when functionally present in the T-Rapa cells; (d) expanding the vector-transduced T-Rapa cells in in vitro culture, and (e) administering the transduced T-Rapa cells into the subject, wherein the T-Rapa cells express the protein encoded by the transgene of interest in the subject and can subsequently reduce one or more symptoms of the lysosomal storage disorder.

Suitable methods of obtaining T-cells (e.g., CD4+ T-cells) from a subject are known in the art including standard outpatient blood draws or apheresis. In one embodiment, obtaining T-cells comprises detecting and isolating CD4+ T-cells from a peripheral blood sample of a subject or suitable donor. Suitable methods of detecting and isolating CD4+ T-cells from peripheral blood are known in the art and include, but are not limited to, for example, flow cytometric cell sorting, including fluorescence-activated cell sorting (FACS), or magnetic separation with the use of magnetic beads that recognize T-cells, including magnet-assisted cell sorting (MACS). In suitable embodiments, antibodies specific to CD4 that may be, in some examples, attached to magnetic beads, and are used to separate CD4+ T-cells from other cells found in peripheral blood. Alternatively, negative selection can be used to deplete the CD4− cells, allowing for the enrichment of CD4+ cells. An advantage of the methods of the current technology are that CD4+ T-cells for use in the methods can be obtained from a peripheral blood sample obtained from an outpatient blood draw and do not require any priming or other treatment steps prior to the isolation of the peripheral blood. In some embodiments, the isolated CD4+ T-cells used in the methods are at least about 70% CD4+ (70% pure), more preferably at least about 75% CD4+ (75% pure), alternatively at least about 80% CD4+ (80% pure), alternatively at least about 85% (85% pure), at least about 90% CD4+ (90% pure), at least about 95% CD4+ (95% pure).

In some embodiments, once the CD4+ T-cells are isolated, the CD4+ T-cells are cultured in vitro to expand the cells.

In some embodiments, once isolated, the isolated CD4+ T-cells are conditioned/treated with rapamycin to form T-Rapa cells. Suitably, the T-cells may be conditioned/treated with rapamycin before transduction with the vector comprising the transgene (e.g. AGA, GAA, ASAH1, and GBA transgene) or other appropriate therapeutic construct. Methods of conditioning T-cells to form T-Rapa cells is known in the art and described in Fowler et al. 2013, the contents of which are incorporated by reference in its entirety. Suitably, the isolated T-cells are cultured in chemically defined medium comprising cytokines and rapamycin in a suitable amount to transform the T-cells into rapamycin resistant T-cells (T-Rapa cells).

Suitable amounts of rapamycin to transform T-cells into T-Rapa cells include, but are not limited to, a concentration of about 0.1 micromolar to about 2 micromolar, (0.1-2 μM), alternatively from about 0.8-1.5 micromolar. Lower concentrations of rapamycin such as 0.1 micromolar can be used; however, lowering the concentration of rapamycin can deteriorate the ability to grow rapamycin-resistant T-cells, and as such, a preferred concentration of rapamycin is about 1 micromolar. Increasing the rapamycin concentration above 1 micromolar has limited feasibility because the drug is not fully solubilized in conventional media above this concentration. As such, concentrations around 1 micromolar are optimal for achievement of the rapamycin resistance (T-Rapa) phenotype.

Once T-Rapa cells are derived, the T-Rapa cells are transduced in vitro with a vector that allows expression of the transgene of interest. Suitable transgenes of interest will depend on the lysosomal storage disorder being treated.

Suitable vectors are known in the art and contain the necessary elements in order for the gene encoded within the vector to be expressed in the host cell. The term “vector” refers to a nucleic acid molecule or genetic construct capable of transporting another nucleic acid to which it has been linked. One type of vector is a “plasmid”, which refers to a circular double stranded DNA loop into which additional DNA segments may be ligated, specifically exogenous DNA segments encoding the targeted protein. Another type of vector is a viral vector, wherein additional DNA segments may be ligated into the viral genome. Certain vectors are capable of autonomous replication in a host cell into which they are introduced. Other vectors can be integrated into the genome of a host cell upon introduction into the host cell, and thereby are replicated along with the host genome (e.g. lentiviral vectors). Moreover, certain vectors are capable of directing the expression of exogenous genes to which they are operatively linked. Such vectors are referred to herein as “recombinant expression vectors” (or simply, “expression vectors” or “vectors”). In general, vectors of utility in recombinant DNA techniques are often in the form of plasmids. In the present specification “vector” include expression vectors, such as viral vectors (e.g., replication defective retroviruses (including lentiviruses), adenoviruses and adeno-associated viruses), which serve equivalent functions. Methods of using viral vectors to transduce cells are known in the art along with the methods of producing the viruses to infect or transduce the cells.

The vectors are heterogeneous exogenous constructs containing sequences from two or more different sources. Suitable vectors include, but are not limited to, plasmids, expression vectors, lentiviruses (lentiviral vectors), adeno- associated viral vectors (rAAV) among others and includes constructs that are able to express the protein encoded by the gene of interest (e.g. AGA transgene). A preferred vector is a lentiviral vector. Suitable methods of making lentiviral vector particles are known in the art and one embodiment is described in the Examples. While specific lentiviral vectors have been used in the examples, the vectors are not limited to these embodiments and any lentiviral vectors or other vectors capable of expressing the transgene of interest are contemplated for use in the practice of the current invention.

A vector can preferably transduce, transform or infect a cell, thereby causing the cell to express the nucleic acids and/or proteins encoded by the vector.

The transgene or transgene of interest may be able to express any protein or enzyme that is associated with a disease or disorder, in some instances, the transgene expresses an enzyme or protein associated with a lysosomal storage disorder.

Suitable lysosomal storage disorders and suitable transgene of interest to target for the lysosomal storage disorders can be found in Table 1. In one embodiment, the lysosomal storage disorder and transgene are selected from Table 1.

| | Transgene of | Sequence ID |
|---|---|---|
| Lysosomal Disorder | interest | (nucleotide) |
| Fabry disease | AGA | SEQ ID NO: 1 |
| Gaucher disease | GBA | SEQ ID NO: 7 |
| Farber disease | ASAH1 | SEQ ID NO: 8 |
| Pompe disease | GAA | SEQ ID NO: 9 |

In one embodiment, the lysosomal storage disorder is selected from the group consisting of Fabry disease, Gaucher disease, Farber disease, and Pompe disease.

In one embodiment, the vector is a lentiviral vector containing a transgene for expression of α-galactosidase A (α-gal A) for the treatment of Fabry disease. As used herein, the term alpha-gal or α-gal A are used interchangeably to refer to α-galactosidase A enzyme (protein).

### Fabry Disease

Suitable methods of cloning the transgene of interest, for example, the codon optimized AGA transgene (SEQ ID NO:1) into an exogenous expression vector (for example a lentiviral vector) are known in the art for producing functional vector to engineer T-Rapa cells to express α-gal A for treatment of Fabry disease. In a preferred embodiment, a suitable expression vector includes, for example, a lentiviral vector, for example, pDY/CO.α-galA (i.e., LV/AGA) (SEQ ID NO:2).

Suitably, the AGA transgene will have at least 80% similarity to the SEQ ID NO:1, alternatively at least 85% sequence similarity to SEQ ID NO:1, alternatively at least 90% sequence similarity to SEQ ID NO:1, alternatively at least 95% sequence similarity to SEQ ID NO:1, alternatively at least 98% sequence similarity to SEQ ID NO:1, alternatively at least 99% sequence similarity to SEQ ID NO:1, alternatively at least 100% sequence similarity to SEQ ID NO:1. Suitable lentiviral vectors for the treatment of Fabry disease include the vector of SEQ ID NO:2, and includes vectors that will have at least 80% similarity to the SEQ ID NO:2, alternatively at least 85% sequence similarity to SEQ ID NO:2, alternatively at least 90% sequence similarity to SEQ ID NO:2, alternatively at least 95% sequence similarity to SEQ ID NO:2, alternatively at least 98% sequence similarity to SEQ ID NO:2, alternatively at least 99% sequence similarity to SEQ ID NO:2, alternatively at least 100% sequence similarity to SEQ ID NO:2.

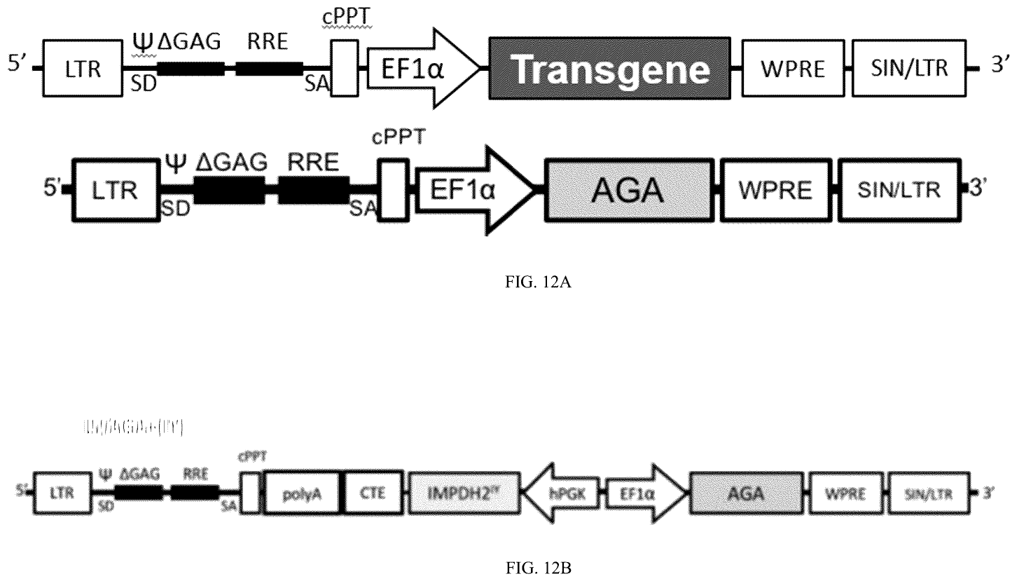

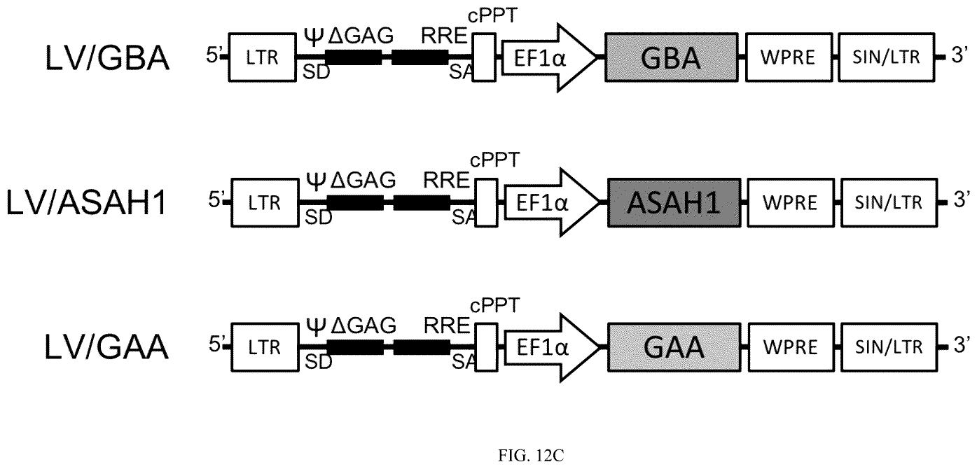

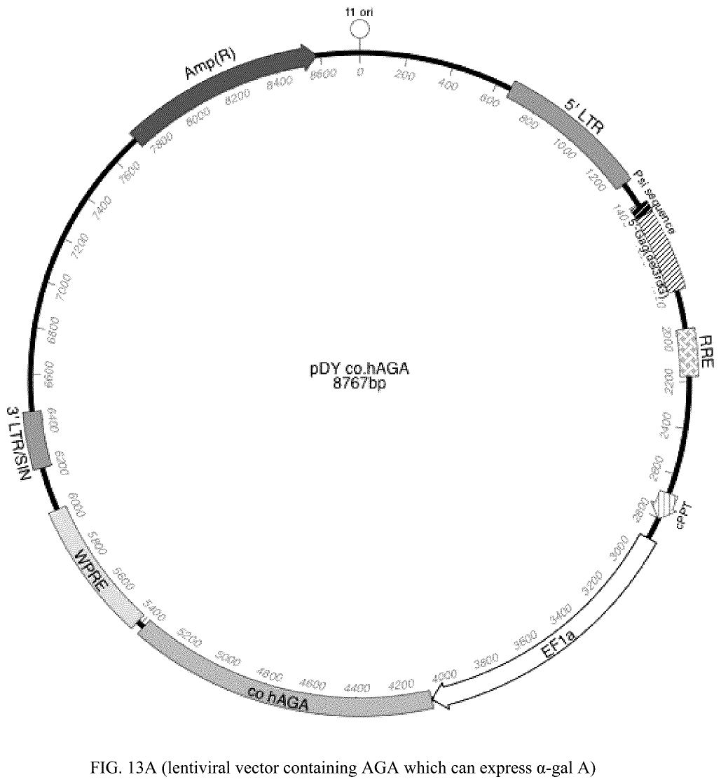

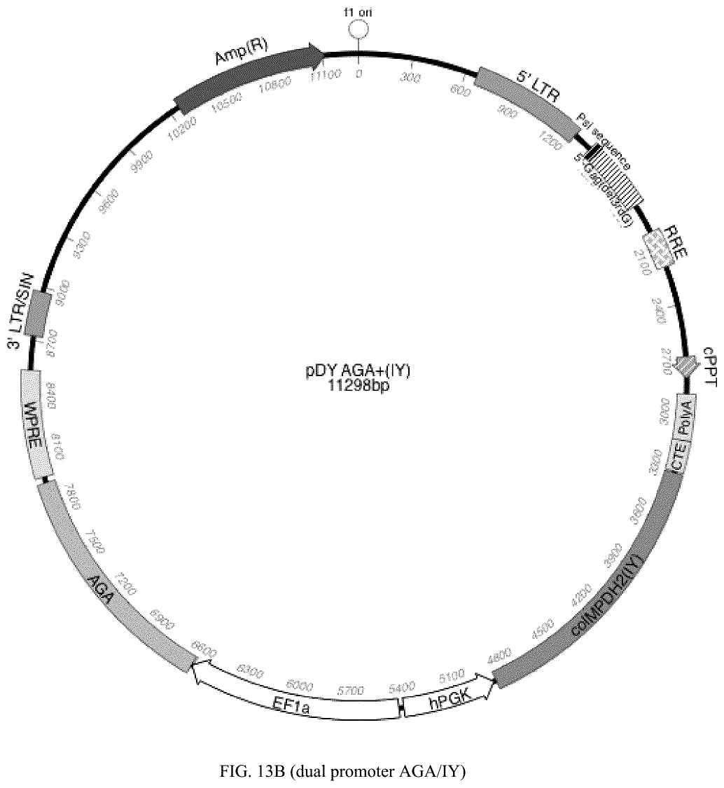

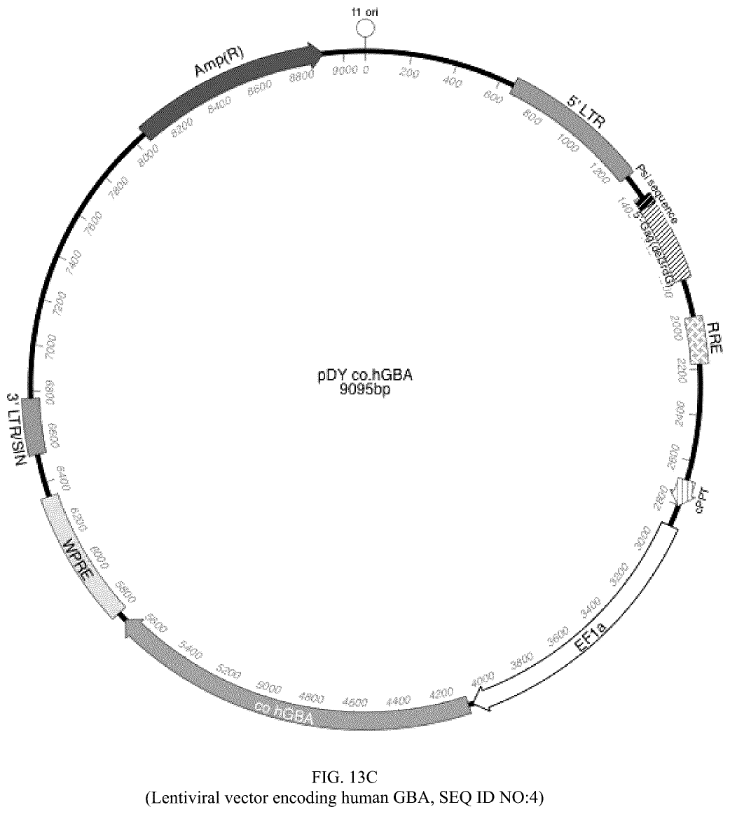

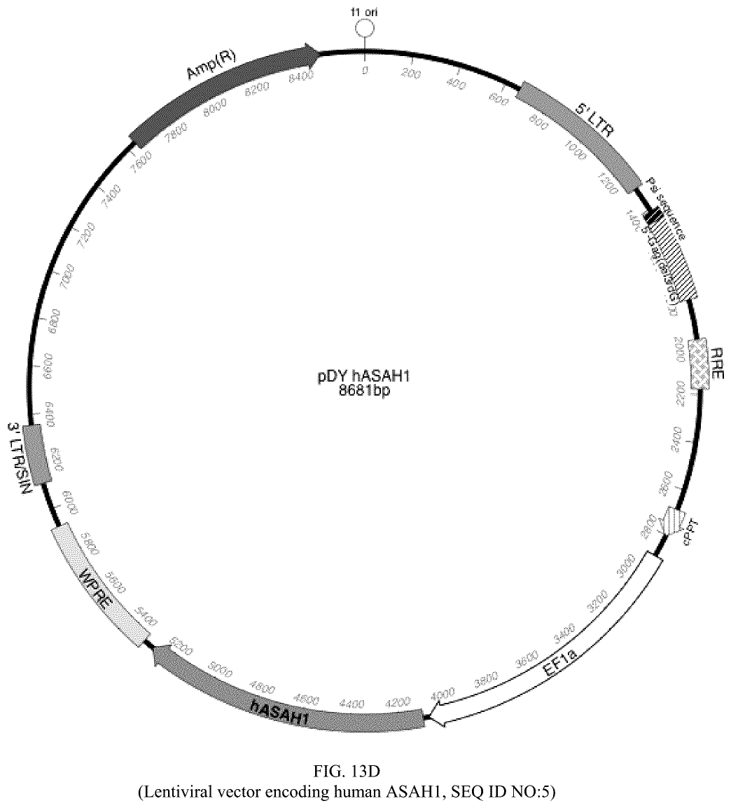

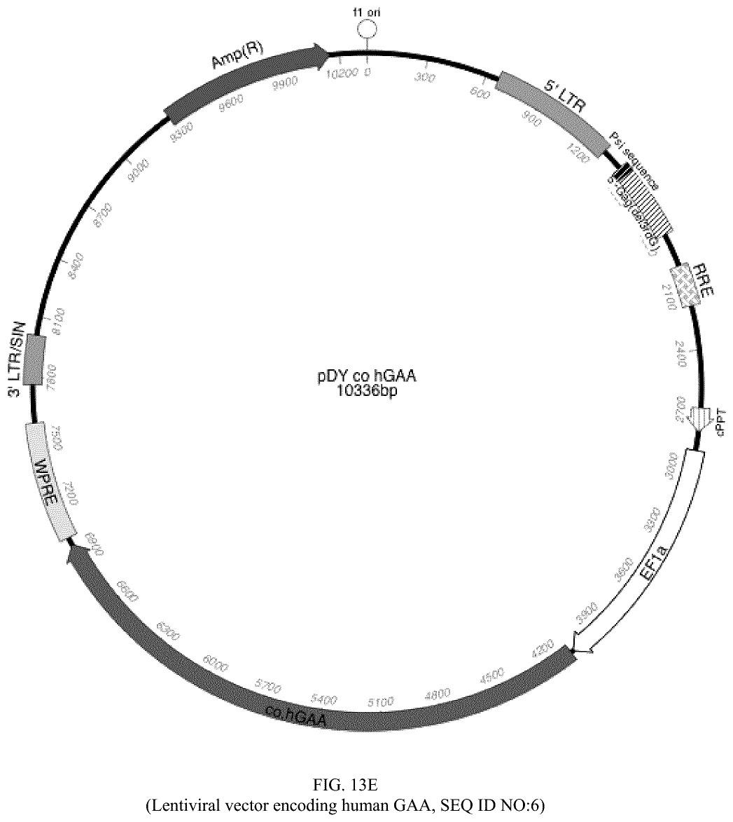

In some embodiments, a dual promoter lentiviral vector may be used that allows for the expression of more than one gene of interest. For example, a dual promoter lentiviral vector may express the transgene of interest to treat one lysosomal storage disorder and another protein of interest to treat the same or different disease. Alternatively, the dual promoter lentiviral vector may be able to express the transgene of interest and a second protein that helps to promote the survival or selection of the transduced T-Rapa cells, either in vitro or in vivo. For exemplary purposes only, one suitable dual promoter vector is LV/AGA+(IY) (SEQ ID NO:3, FIG. 13B), as described in Provisional Application No. 62/516,022, the contents of which are incorporated by reference in its entirety. In one suitable embodiment, the T-Rapa cells are transduced with a lentiviral vector as depicted in FIG. 12. In another embodiment, the T-Rapa cells are transduced with a dual promoter lentivirus vector that expresses α-galactosidase A and a mutant form of inosine-5′-monophosphate dehydrogenase 2 (IMPDH2(IY)) (e.g., vector encoded by SEQ ID NO:3). Use of such a lentiviral vector will allow for further enrichment of the transduced T-Rapa cells in vivo in the subject by the treatment of the subject with an effective amount of mycophenolate mofetil (MMF) or mycophenolic acid (MPA) sufficient to enrich the population of lentivirus vector transduced T-Rapa cells in the subject.

The primary consequence of MPA/MMF administration is T and B cell depletion. By expressing IMPDH2(IY), T-Rapa cells that were transduced are resistant to MPA/MMF. Treatment with low doses of MMF can increase the number of therapeutic T-Rapa, without affecting the original engraftment, while causing minimal or no toxicity. This, in turn, increases the total number of circulating cells that are expressing and secreting the transgene, for example, α-galactosidase A, which can lead to better correction of the disease. The current method gives a way to enrich for transduced cells in vivo and allows some gating as to how selective and strong that enrichment is depending on the administration of the MMF. The present methods also allow for cells harboring this lentiviral vector to be enriched for even years down the road to renew the correcting cell population expressing the transgene of interest.

In suitable embodiments, the MMF is administered at an effective dosage. An “effective dosage” refers to a dosage that allows for selective enrichment of T-Rapa cells that express the transgene via the lentiviral vector with minimal side effects. In one embodiment, the effective dosage is a low dosage. Suitable low dosages include, but are not limited to, for example, 0.1-5 mg/kg body weight given TID (three times a day), alternatively include from about 0.1-3 mg/kg body weight given TID. Alternatively, the effective dose may include higher doses of MMF. Suitable higher dosage of MMF for practice of this invention include MMF in an amount of about 5-10 mg/kg body weight TID (three times a day), alternatively 1000 mg given BID (two times a day). Suitably, an “effective amount” of MMF will result in a blood concentration within the subject of about 0.4 to about 2 μM free mycophenolic acid (MPA). Suitable dosages to obtain this blood concentration are readily determined by a physician treating the subject. MMF may also be substituted for mycophenolic acid (MPA) formulations (Myfortic, Novartis, or approved generic).

### Gaucher Disease

In some embodiments, the lysosomal storage disorder is Gaucher disease. In Gaucher disease, mutations in the GBA gene greatly reduce or eliminate the activity of β-glucocerebrosidase, which breaks down waxy substances of the lipid class glycosphingolipids called glucocerebrosides into a sugar (glucose) and ceramide, another sphingolipid. Without enough of this enzyme, glucocerebroside and related substances can build up to toxic levels within cells. Tissues and organs are damaged by the abnormal accumulation and storage of these substances, causing the characteristic features of Gaucher disease. Suitable embodiments of the present invention provide for T-Rapa cells expressing GBA for the treatment of Gaucher disease. In one embodiment, a lentiviral vector comprises the transgene (e.g., GBA transgene) that allows for expression of β-Glucocerebrosidase (e.g. GBA transgene found in SEQ ID NO:7) in the transduced cells. Suitably, the GBA transgene will have at least 80% similarity to the SEQ ID NO:7, alternatively at least 85% sequence similarity to SEQ ID NO:7, alternatively at least 90% sequence similarity to SEQ ID NO:7, alternatively at least 95% sequence similarity to SEQ ID NO:7 alternatively at least 98% sequence similarity to SEQ ID NO:7, alternatively at least 99% sequence similarity to SEQ ID NO:7, alternatively at least 100% sequence similarity to SEQ ID NO:7.

In one embodiment, the vector is a lentiviral vector that comprises the transgene GBA of SEQ ID NO:7 or a sequence with at least 80% identity to SEQ ID NO:7.

Suitable sequence for the lentiviral vector comprising GBA is found in SEQ ID NO:4 and depicted in FIG. 13C, or a sequence that will have at least 75% similarity to SEQ ID NO:4, alternatively at least 80% similarity to the SEQ ID NO:4, alternatively at least 85% sequence similarity to SEQ ID NO:4, alternatively at least 90% sequence similarity to SEQ ID NO:4, alternatively at least 95% sequence similarity to SEQ ID NO:4, alternatively at least 98% sequence similarity to SEQ ID NO:4, alternatively at least 99% sequence similarity to SEQ ID NO:4, alternatively at least 100% sequence similarity to SEQ ID NO:4. Other suitable vectors that encode for the expression of the β-Glucocerebrosidase protein are contemplated herein.

### Farber Disease

In some embodiments, the lysosomal storage disorder is Farber disease (also known as Farber's lipogranulomatosis, ceramidase deficiency, “Fibrocytic dysmucopolysaccharidosis,” and “Lipogranulomatosis”) and the transgene ASAH1 expresses N-Acylsphingosine Amidohydrolase 1 or acid ceramidase (used interchangeably herein). Farber disease is an extremely rare autosomal recessive lysosomal storage disorder marked by a deficiency in the enzyme acid ceramidase that causes an accumulation of a waxy class of lipids known as sphingolipids, in particular ceramide, leading to abnormalities in the joints, liver, throat, visceral tissues and central nervous system. Suitable embodiments provide T-Rapa cells expressing N-Acylsphingosine Amidohydrolase 1 for the treatment of Farber disease. Suitable vectors, preferably a lentiviral vector, are used to express N-Acylsphingosine Amidohydrolase 1 within the T-Rapa cells. As used in the present invention, a suitable vector, preferably a lentiviral vector can be used to express N-Acylsphingosine Amidohydrolase 1 in the T-Rapa cells. For example, a suitable vector can express N-Acylsphingosine Amidohydrolase 1 using the ASAH1 transgene of SEQ ID NO:8 or a sequence having 80% similarity to SEQ ID NO:8. Suitably, the ASAH1 transgene will have at least 80% similarity to the SEQ ID NO:8, alternatively at least 85% sequence similarity to SEQ ID NO:8, alternatively at least 90% sequence similarity to SEQ ID NO:8, alternatively at least 95% sequence similarity to SEQ ID NO:8, alternatively at least 98% sequence similarity to SEQ ID NO:8, alternatively at least 99% sequence similarity to SEQ ID NO:8, alternatively at least 100% sequence similarity to SEQ ID NO:8.

A suitable lentiviral vector includes the vector depicted in FIG. 13D and in SEQ ID NO:5. Suitable sequence for the lentiviral vector comprising ASAH1 transgene found in SEQ ID NO:5, or a sequence that will have at least 75% similarity to SEQ ID NO:5, alternatively at least 80% similarity to the SEQ ID NO:5, alternatively at least 85% sequence similarity to SEQ ID NO:5, alternatively at least 90% sequence similarity to SEQ ID NO:5, alternatively at least 95% sequence similarity to SEQ ID NO:5, alternatively at least 98% sequence similarity to SEQ ID NO:5, alternatively at least 99% sequence similarity to SEQ ID NO:5, alternatively at least 100% sequence similarity to SEQ ID NO:5. Other suitable vectors that encode for the expression of the N-Acylsphingosine Amidohydrolase 1 protein are contemplated herein.

### Pompe Disease

In another embodiment, the present invention provides vectors and T-Rapa cells expressing acid α-glucosidase (encoded by the GAA transgene) for the treatment of Pompe disease. Pompe disease is an inherited disorder resulting from the inability to breakdown a complex sugar called glycogen in lysosomes of the body's cells resulting in accumulation of glycogen in certain organs and tissues, especially muscles, which impairs their ability to function normally. Mutations within the GAA gene cause Pompe disease as the GAA gene provides instructions for producing an enzyme called acid α-glucosidase (also known as acid maltase). This enzyme is active in lysosomes which serve as recycling centers within cells. The enzyme normally breaks down glycogen in lysosomes into a simpler sugar called glucose, which is the main energy source for most cells. In some embodiments, T-Rapa cells expressing acid α-glucosidase are used to treat a subject having Pompe disease. As described above, vectors, preferably lentiviral vectors can be used to express acid α-glucosidase via the GAA transgene within the T-Rapa cells for subsequent secretion by them. In one embodiment, the vectors, preferably lentiviral vectors comprise the GAA transgene of SEQ ID NO:9 or a sequence at least 80% similar to SEQ ID NO:9. Suitably, the GAA transgene will have at least 80% similarity to the SEQ ID NO:9,alternatively at least 85% sequence similarity to SEQ ID NO:9, alternatively at least 90% sequence similarity to SEQ ID NO:9, alternatively at least 95% sequence similarity to SEQ ID NO:9, alternatively at least 98% sequence similarity to SEQ ID NO:9, alternatively at least 99% sequence similarity to SEQ ID NO:9, alternatively at least 100% sequence similarity to SEQ ID NO:9.

In one embodiment, the suitable lentiviral vector is shown in FIG. 13E and SEQ ID NO:6. Suitable sequence for the lentiviral vector comprising GAA is found in SEQ ID NO:6, or a sequence that will have at least 75% similarity to SEQ ID NO:6, alternatively at least 80% similarity to the SEQ ID NO:6, alternatively at least 85% sequence similarity to SEQ ID NO:6, alternatively at least 90% sequence similarity to SEQ ID NO:6, alternatively at least 95% sequence similarity to SEQ ID NO:6, alternatively at least 98% sequence similarity to SEQ ID NO:6, alternatively at least 99% sequence similarity to SEQ ID NO:6, alternatively at least 100% sequence similarity to SEQ ID NO:6. Other suitable vectors that encode for the expression of a form of acid α-glucosidase protein are contemplated herein.

Other lysosomal disorders listed in Table 1 are contemplated to be treated by the methods described herein.

“Percentage of sequence identity” or “sequence similarity” is determined by comparing two optimally aligned sequences over a comparison window, wherein the portion of the polynucleotide sequence in the comparison window may comprise substitutions, or additions or deletions (i.e., gaps) as compared to the reference sequence (which does not comprise substitutions, additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “similarity” of polynucleotide sequences means that a polynucleotide comprises a sequence that has at least 80% sequence identity. Suitable sequence similarity allows for small changes in the transgene that do not affect the function of the protein expressed by the transgene. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using programs such as BLAST using standard parameters. These values can be appropriately adjusted to determine corresponding identity of proteins encoded by two nucleotide sequences by taking into account codon degeneracy, amino acid similarity, reading frame positioning and the like.

Suitable amounts of lentivirus able to transduce T-Rapa cells include, for example, using a MOI (multiplicity of infection) of 1-100, preferably an MOI of 1-30, alternatively 1-60. The T-Rapa cells may be exposed to the lentivirus for 10-24 hours, suitably about 12-16 hours. The T-Rapa cells may be transduced consecutively 1-3 times for exposure times listed herein, suitably 1 time. Cytokines may be added to the culture medium during transduction. After transduction, the cells can be either transferred back into the patient or cryopreserved for later transplantation, or a combination of both. In some instances, the transduced cells may be cultured for a number of days before being transferred or cryopreserved. Suitable methods of cryopreservation are known in the art.

Other suitable vectors are known in the art and can be used to transduce the cell including AAV vectors, and the like. Any vector that is able to allow for stable expression of the enzyme encoded by the transgene of interest is contemplated for use herein.

In some embodiments, the transduced T-Rapa cells are expanded in culture and cryopreserved at various stages of culture. Suitable methods of cryopreservation include, but are not limited to, suspending the cells in a cryopreservation medium and storing the cells at −80° C. to −196° C., preferably below −80° C. Suitable cryopreservation media are known in the art and may comprise some combination of base medium, cryopreservative (e.g., DMSO) and a protein source. For example, a suitable cryopreservation medium may comprise complete medium and 10% glycerol, complete medium containing 10% DMSO (dimethlysulfoxide), or 45% cell-conditioned medium with 45% fresh medium and 10% glycerol or DMSO. In alternative embodiments, the cryopreservation medium may be serum free, for example, comprises 46.25% cell-conditioned serum-free medium with 46.25% fresh serum-free medium and 7.5% DMSO.

Suitable chemically defined medium for culturing T-cells are known in the art and include, but are not limited to, commercial nutrient-rich media such as X-Vivo 20. Suitably, the chemically defined medium is further supplemented with cytokines. Preferably, in one embodiment, recombinant human IL-2 (rhu IL-2) and recombinant human IL-4 (rhu IL-4) cytokines are used to supplement the medium. Suitable amount of the recombinant cytokines include about 10-100 IU/mL of IL-2, preferably about 20 IU/mL of IL-2 and about 500-2000 IU/mL of IL-4, preferably about 1000 IU/mL IL-4.

In some embodiments, the transduced T-Rapa cells are expanded in vitro. During expansion, the transduced T-Rapa cells may be cultured in chemically defined medium supplemented with cytokines as described herein. Suitably, the transduced T-Rapa cells may be cultured for at least one day, and suitably may be cultured for at least 2 weeks, alternatively at least 4 weeks, alternatively at least 6 weeks.

The transduced T-Rapa cells may be maintained and expanded in vitro in culture for at least 5 passages, alternatively at least 10 passages, alternatively at least 15 passages, alternatively at least 20 passages. The transduced T-Rapa cells may be cryopreserved at any passage after transduction.

The present disclosure contemplates populations of transduced T-Rapa cells that express the protein encoded by a transgene of interest and any methods of use thereof. For example, the present disclosure provides a population of transduced T-Rapa cells that express a protein encoded by the transgene of interest. In one embodiment, the disclosure provides a population of transduced T-Rapa cells that express α-gal A. In another aspect, the discourse provides a population of transduced T-Rapa cells that express β-glucocerebrosidase. In another aspect, the disclosure provides a population of transduced T-Rapa cells that express acid ceramidase. In another aspect, the disclosure provides a population of transduced T-Rapa cells that express acid α-glucosidase.

Suitably, the transduced T-Rapa cells are administered into the subject having a lysosomal storage disorder in an amount effective to reduce one or more symptoms of the lysosomal storage disorder (e.g. Fabry disease). Suitable methods of administering the transduced T-Rapa cells are known in the art, and include, but are not limited to, intravenous injection and transfusion.

The transduced T-Rapa cells may be administered at least once, and suitably will be administered at subsequent times at which increased expression of the enzyme or protein of interest (e.g. α-gal A expression) are needed to treat one or more symptom of the lysosomal storage disorder (e.g. Fabry disease). A skilled artisan familiar with lysosomal storage disorders will appreciate monitoring enzyme (e.g. α-gal A) production and the necessity for additional administrations.

The term “subject” or “patient” are used interchangeably and refer to a mammalian subject, for example, a mouse, a rat, a monkey, a human, etc. In a preferred embodiment, the subject is a human. It is contemplated that the subject or patient may have already been treated with one or more therapies for the lysosomal storage disorder before undergoing the treatment contemplated herein. For example, patients treated with exogenous enzymes or by prior methods of using transduced HSC cells are contemplated as subjects for use of the present invention.

The host cell is suitably a T-cell, for example CD4+ T-cells. Although the examples provided here describe the use of CD4+ T-cells, in some embodiments, it may be advantageous to manufacture a mixed population of CD4+ and CD8+ T-cells that secrete the therapeutic protein (transgene of interest). Further, in one embodiment, the T-cells are skewed toward Th2 cytokine phenotype, in some embodiments, it may be advantageous to manufacture T-cells skewed towards other phenotypes such as Th1, Th17, or a regulatory T-cell subset.

Suitable methods of producing a population of transduced T-Rapa cells are provided herein.

In some embodiments, the transduced T-Rapa cells are administered to the subject with a pharmaceutically acceptable carrier or excipient.

A “pharmaceutically acceptable carrier” means any conventional pharmaceutically acceptable carrier, vehicle, or excipient that is used in the art for production and administration of compositions to a subject. Pharmaceutically acceptable carriers are typically non-toxic, inert, solid or liquid carriers which are physiologically balanced. Typically, buffered saline or other saline solutions are physiologically acceptable carriers. Water is not contemplated as a suitable physiologically acceptable carrier. In some embodiments, additional components may be added to preserve the structure and function of the T-Rapa cells of the present invention, but are physiologically acceptable for administration to a subject.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

Aspects of the present disclosure that are described with respect to methods can be utilized in the context of the compositions of matter or kits discussed in this disclosure. Similarly, aspects of the present disclosure that are described with respect to compositions of matter can be utilized in the context of the methods and kits, and aspects of the present disclosure that are described with respect to kits can be utilized in the context of the methods and compositions of matter.

The invention will be more fully understood upon consideration of the following non-limiting examples.

### EXAMPLES

### Example 1: Production of Lentiviral Vector

### LV Construction

A DNA fragment comprising the cDNA of the human GLA gene encoding α-gal A was synthesized by GenScript. The cDNA of the GLA gene was codon-optimized for enhanced expression in human cells. The synthesized DNA fragment was sub-cloned into the 3′ self-inactivating (3′SIN), HIV-1-based, lentiviral backbone plasmid pDY.cPPT-EF1a-MCS-WPRE previously generated in our laboratory between the EcoRI and XmaI restriction sites in the multiple cloning site (MCS). Following this we used site-directed mutagenesis using the Q5 site-directed mutagenesis kit (New England Biolabs) to edit the sequence upstream of the cDNA to create an optimal Kozak consensus sequence. These steps generated the plasmid pDY/CO.α-gal A (SEQ ID NO:2) which was used for all LV preparations in our studies. The plasmid sequence covering the proviral region was verified by DNA sequencing. This method was described in and is paraphrased from Huang et al., 2017, the contents of which are incorporated by reference in its entirety. Purification of Research-Grade and Near-Clinical-Grade LV along with Functional Titer Analyses

Research-grade LV/AGA was prepared in our laboratory as described previously (Wang, J. C., Felizardo, T. C., Au, B. C., Fowler, D. H., Dekaban, G. A., and Medin, J. A. (2013). Engineering lentiviral vectors for modulation of dendritic cell apoptotic pathways. Virol. J. 10, 240.) Near-clinical-grade LV/AGA was produced, which meets current GMP requirements for potential human clinical trial use under an investigational new drug (IND) submission.

The LV particles were produced using HEK293T packaging cells. The packaging cells were expanded to a 4-L culture volume and transiently co-transfected with the LV packaging plasmids (pCMVΔR8.91 (packaging plasmid) and pMD.G (VSV-G envelope encoding plasmid)) and transfer plasmid (pDY/CO.α-gal A). The sequences of those plasmids that were expanded were verified by DNA sequencing. Culture supernatant was harvested twice, yielding a total of 8 L of unconcentrated LV-containing supernatant. The LV-containing supernatant was further purified by Mustang Q ion exchange chromatography, concentrated by tangential flow filtration, and buffer-exchanged into 100 mL GMP-grade Lonza X vivo 20 cell growth medium.

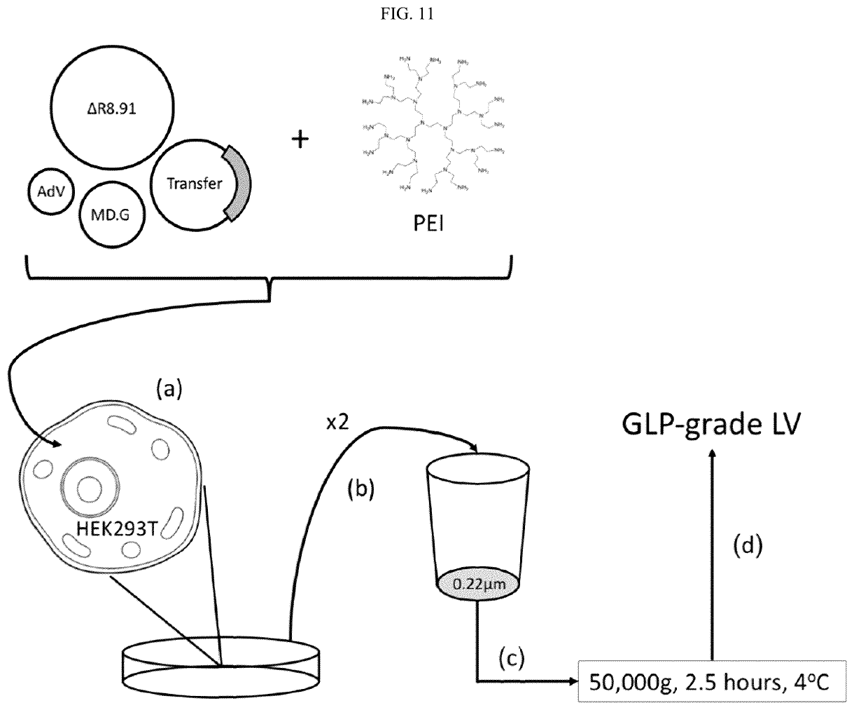

Vesicular stomatitis virus glycoprotein-pseudotyped lentiviruses (VSVg-LVs), were generated. Briefly, HEK293T cells were seeded in 15 cm culture dishes and transfected 24 hours later with LV packaging plasmids and transfer plasmid. 16-17 hours later media on the cells was exchanged for fresh media. 24 hours later culture supernatant was collected and replaced with fresh media. A second collection was performed another 24 hours later. The culture supernatant was filtered through 0.22 μm vacuum-assisted filters, and ultra-centrifuged at 50,000 g for 2 and half hours. Residual liquid was removed from the viral pellets, and these were then resuspended in Lonza X Vivo 20 and stored at −80° C. until use. Viral supernatants were harvested 24 and 48 hours later and concentrated by ultracentrifugation at 50,000 g for 2 hours as depicted in FIG. 11. Viral stocks were resuspended in lymphocyte culture medium (Lonza X Vivo 20) and stored at −80° C. until use.

Sample vials of the final concentrated vector product underwent QC analyses, including vector identity confirmation by Southern blot analysis and titer by p24 ELISA, along with testing for aerobic and anaerobic sterility, mycoplasma levels, endotoxin levels, and residual DNA benzonase levels.

We performed infectious titer testing of all LV preparations by transduction of HEK293T cells using serial dilutions of the vector followed by measurement of average viral copy number per cell using quantitative real-time PCR analysis.

### Example 2: Transduced T-Rapa Cell Manufacturing

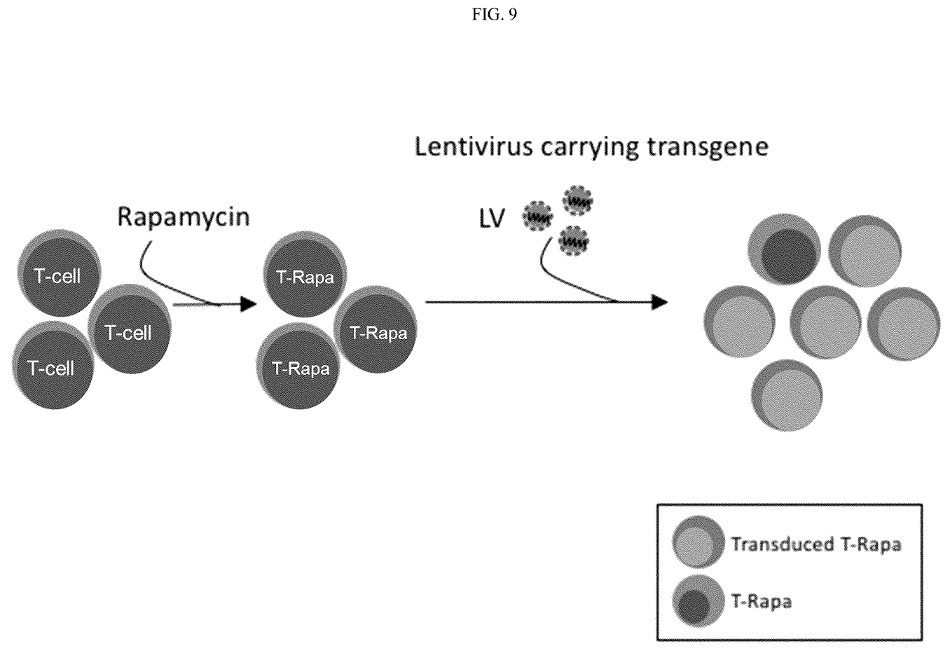

A schematic of making transduced T-Rapa cells is depicted in FIG. 9. Donor lymphocytes are collected by a 10-liter steady-state apheresis performed prior to stem cell mobilization. CD4 cells were positively selected (Miltenyi; CliniMACS® device, or laboratory equivalent) and co-stimulated (tosylated magnetic beads [Dynal] conjugated with GMP-grade anti-CD3 [OKT3; Ortho] and GMP-grade anti-CD28 9.3 antibodies [3:1 bead:cell ratio]). Alternatively, donor lymphocytes were obtained as CD34-depleted flow-through from CD34+ cliniMACS procedure from stem cell mobilized donors. CD4+ cells were obtained using magnetic enrichment as previously described.

Purified CD4+ cells will be cultured in polyolefin bags (Baxter) using X-VIVO 20 media (Lonza), 5% donor plasma, recombinant human (rhu) IL-4 (1000 I.U./mL; Schering), rhu IL-2 (20 I.U./mL; Chiron) and Sirolimus® oral solution (Wyeth; 1 μM) and anti-CD3/CD28 beads (3:1). After 3 days, T-cells will be washed and transduced with lentivirus vector able to express α-gal A at MOI of 30-60. After 18 hours, T-cells will be washed and propagated in supplemented X-VIVO 20 media without rapamycin. On day 6, beads will be removed; T-cells will be washed to remove cytokines, and then cryopreserved. All infused T-Rapa products will meet release criteria, which include: CD4 cell purity >70% (median CD4 purity was 99%), viability >70% (median viability was 95%), absence of bacterial and fungal growth, absence of endotoxin content by limulus assay, negative mycoplasma test, and <100 magnetic beads per 3×10⁶ cells. T-Rapa cells are cultured, expanded and cryopreserved for use.

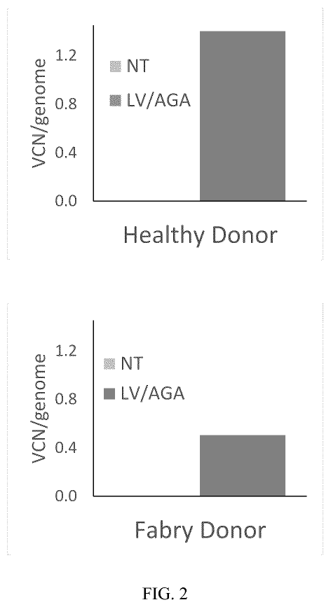

As depicted in FIG. 2, both T-Rapa cells derived from a healthy donor (ND) and Fabry donor (FD) were efficiently transduced with the lentiviral vector.

### Example 3: Expression of α-gal A in T-Rapa Cells Transduced With LV

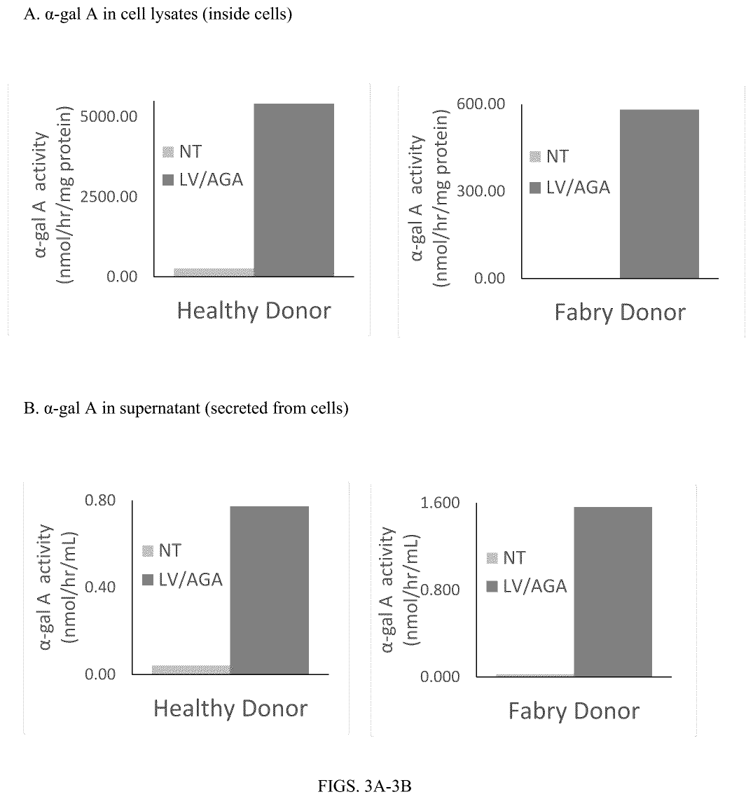

Levels of α-gal A were determined for the transduced T-Rapa cells from healthy and Fabry donors. The specific α-gal A activity was determined by fluorometric assay as previously described (Yoshimitsu et al. 2004, PNAS: 942540-2544). Briefly, plasma or cell/organ protein extracts were incubated with 4-methylumbelliferyl-α-D-galactopyranoside (5 mmol/L) in presence of the α-N-acetylgalactosaminidase inhibitor, N-acetyl-D-galactosamine (100 mmol/L) (Sigma Aldrich, St. Louis, MO). The product of the enzymatic reaction was quantified by comparison with known concentrations of 4-methylumbelliferone. Each measurement was assessed in triplicate and normalized to total protein concentration (BCA Protein Assay Kit; Pierce, Rockford, IL). Results are shown in FIGS. 3A and 3B, depicting the α-gal A content in cell lysates (A) and supernatants (B).

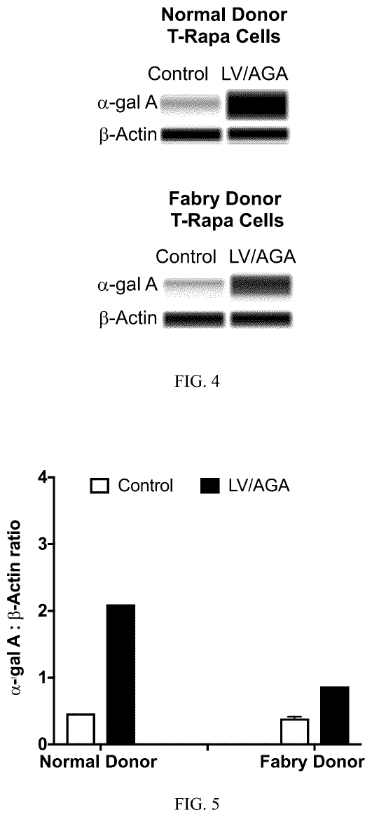

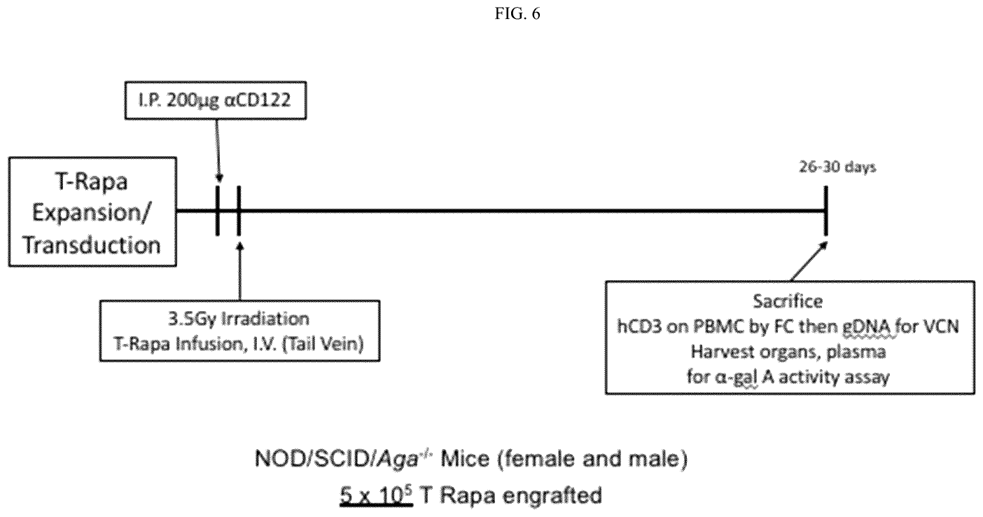

Levels of α-gal A were also detected by Wes™ Simple Western System as shown in FIGS. 4 and 5.

### Example 4: In Vivo Treatment of a Mouse Model of Fabry Disease.

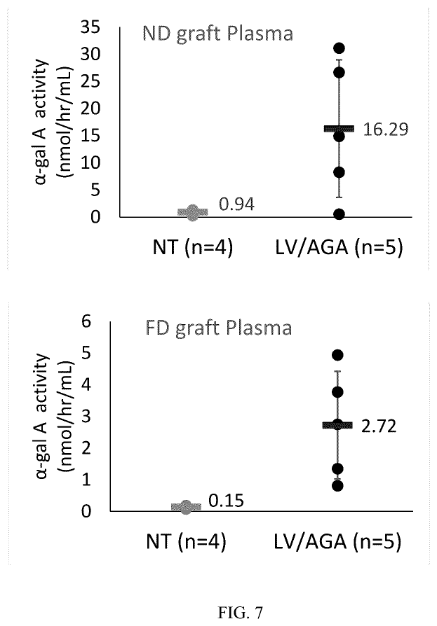

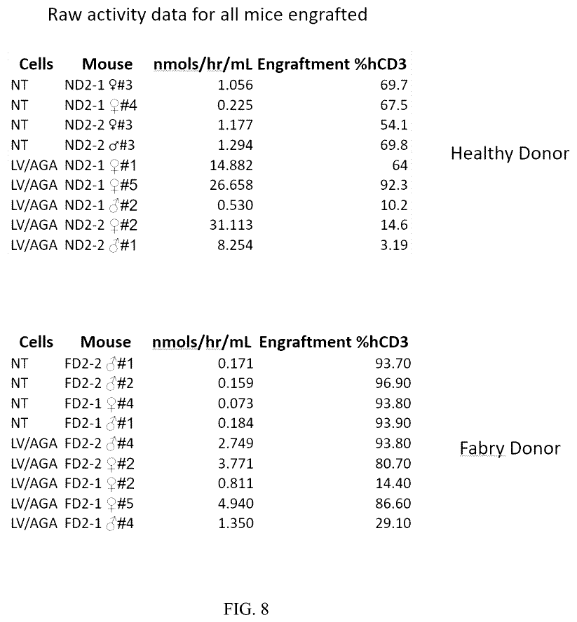

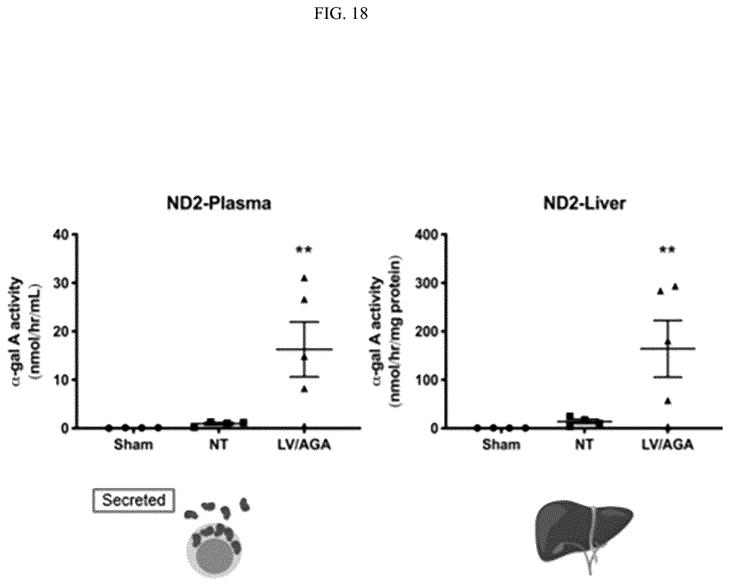

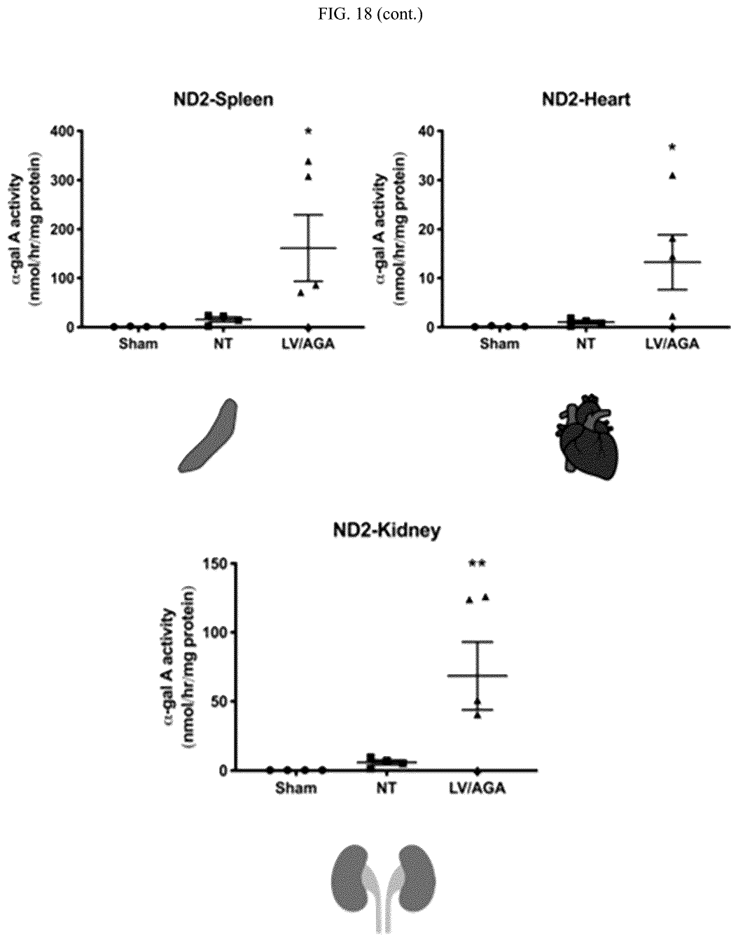

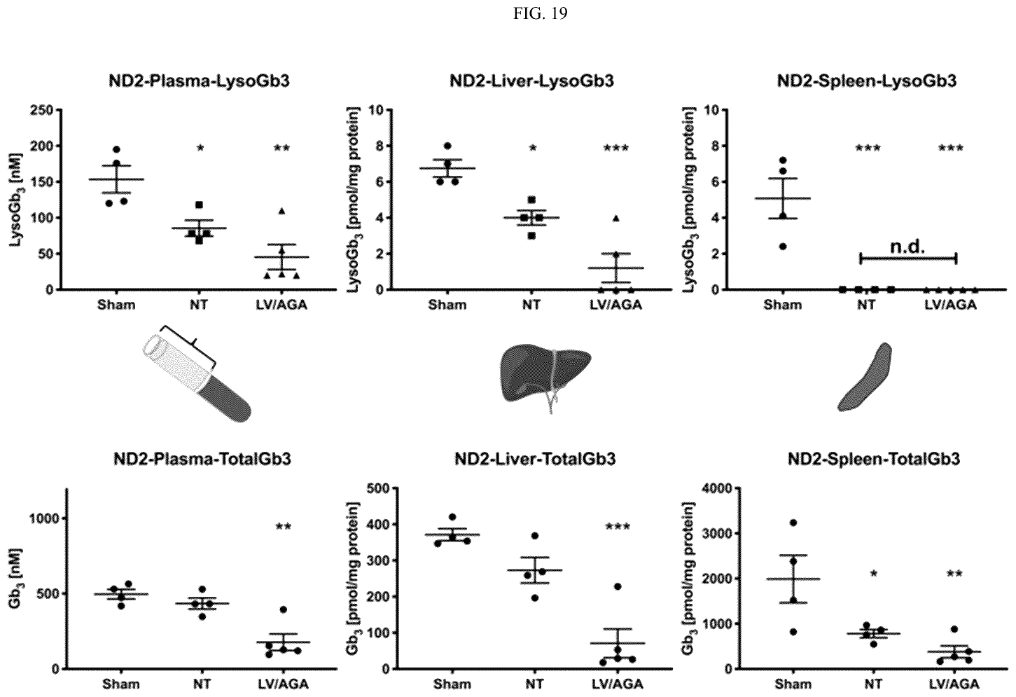

α-gal A-deficient and immunocompromised Fabry mice (NOD/SCID/Aga^{-/-}) as described previously (Pacienza et al. 2012) were used to test the in vivo efficacy of using transduced T-Rapa cells for treatment of Fabry disease. NOD/SCID/Aga^{-/-} mice were engrafted with 5×10⁵ transduced human T-Rapa cells from either healthy or Fabry donors as depicted in FIG. 6. α-gal A activity in the engrafted animals was assayed 4 weeks after xeno-transplant. Liver, spleen heart, kidney, plasma and PB-WBCs were collected. PB-WBCs were assayed by flow cytometry and VCN for presence of transduced T-Rapa cells. Plasma was assessed for α-gal A activity, the results shown in FIG. 7 and the raw data shown in FIG. 8. FIG. 18 demonstrates α-gal A activity is detectable in the collected organs in vivo 4 weeks after xenograft of transduced healthy donor T Rapa (n=4-5).

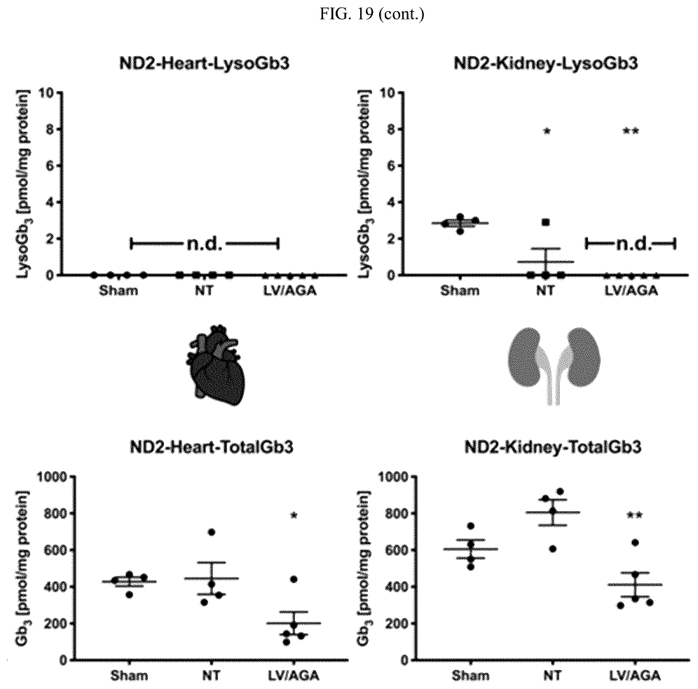

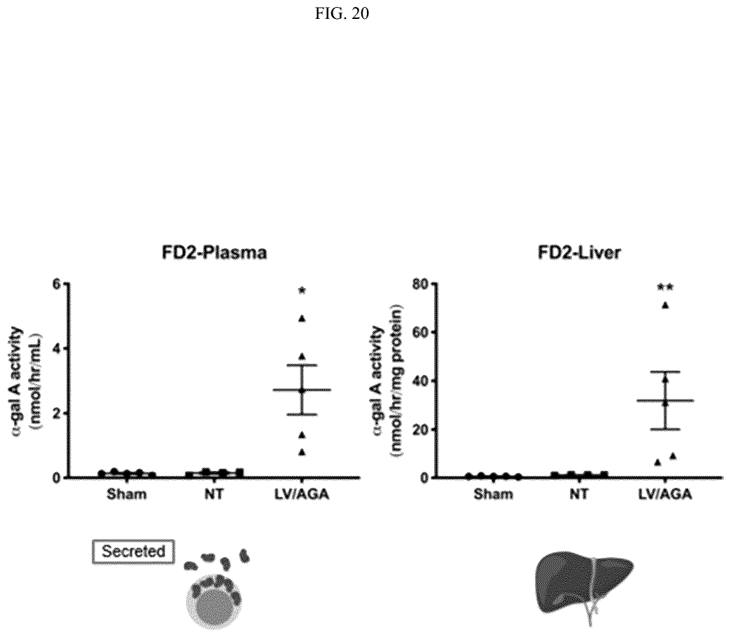

Further, the ability of the transduced T-Rapa cells to reduce substrate in vivo was also assayed. Globotriaosylceramide (Gb₃), the primary substrate that accumulates in Fabry mice, and globotriaosylsphingosine (lyso-Gb₃) were quantified in plasma and tissue homogenates by UPLC-MS/MS after treatment with the healthy donor transduced T-Rapa cells. As shown in FIG. 19, substrate is reduced 4 weeks after transplant of transduced healthy donor T Rapa (n=4-5).

LV/AGA vector-transduced T-Rapa cells derived from healthy and Fabry donors produce and secrete active enzyme in vivo.

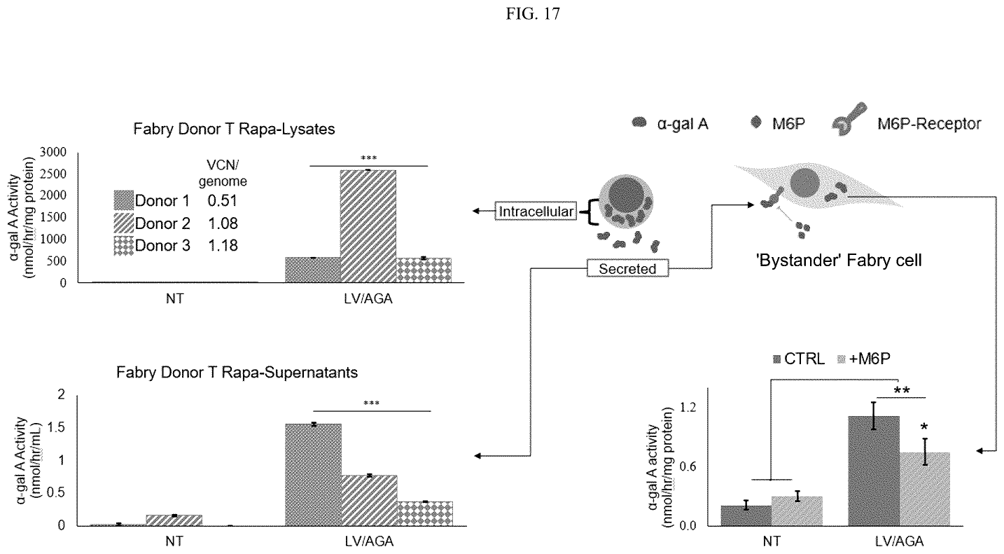

CD4+ T cells derived from Fabry patients were also transduced with the lentiviral vector encoding α-gal A as described above. First, we confirmed that the T-Rapa cells from 3 Fabry patients showed α-gal A activity, and measured enzyme activities both within the cell and in the cellular supernatants as shown in FIG. 17. We then treated immortalized Fabry patient-derived fibroblasts with T-Rapa-conditioned supernatant for 6 hours with or without 1 mM soluble mannose-6-phosphate to see if enzyme produced from T-Rapa cells can be taken up (FIG. 17).

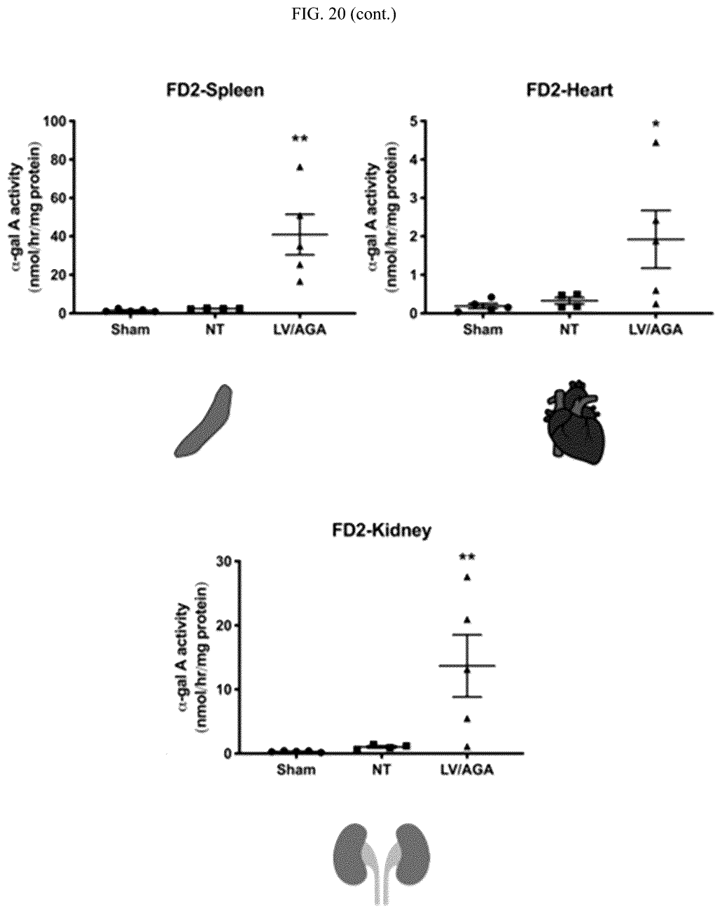

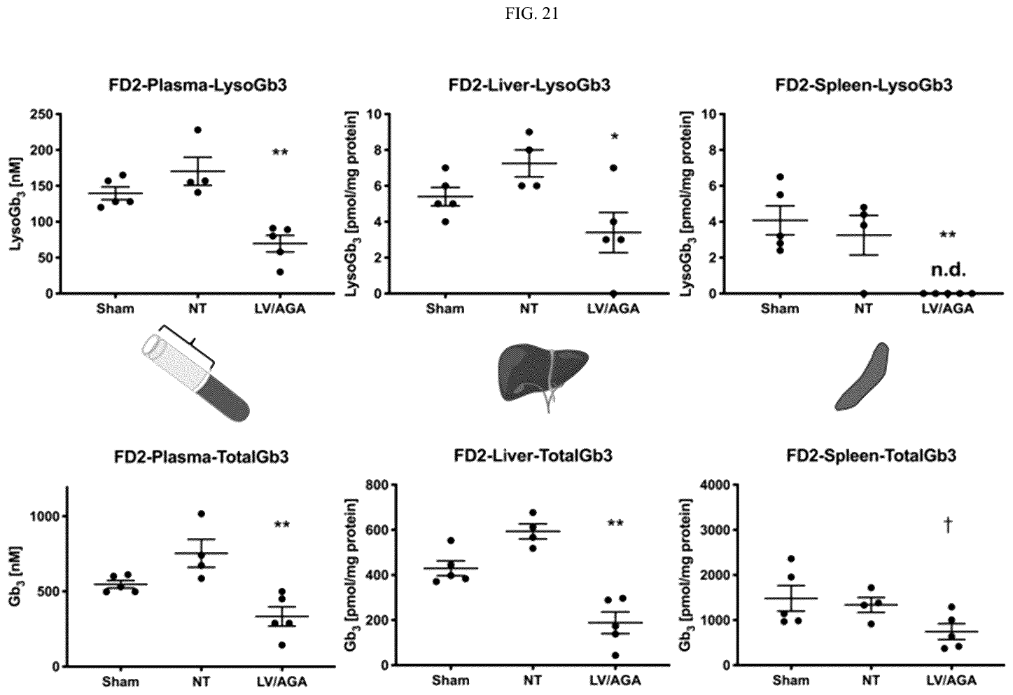

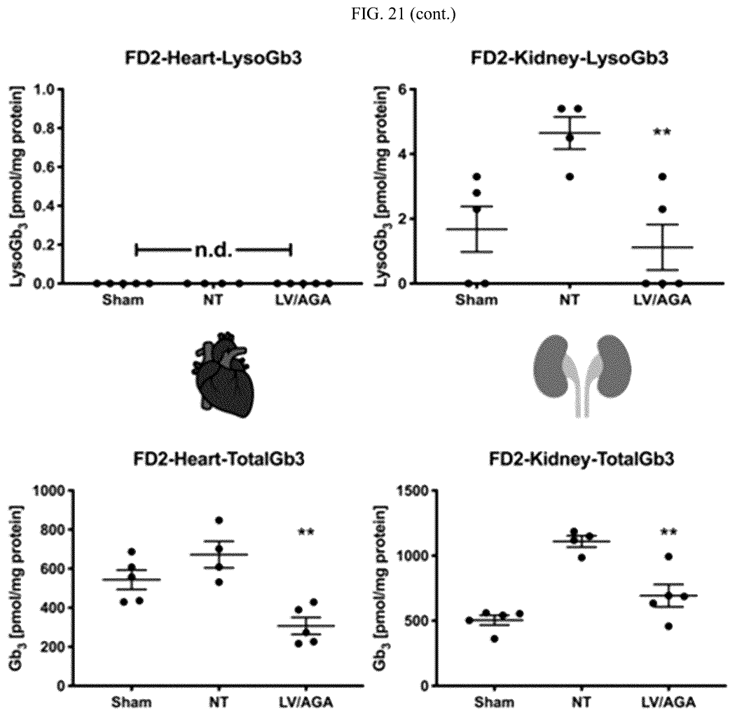

These transduced T-Rapa cells derived from Fabry patients were also engrafted into the immunocompromised Fabry mouse model, and the α-gal A activity was measured. As shown in FIG. 20, α-gal A is detectable in vivo 4 weeks after xenograft of transduced Fabry donor T-Rapa (n=4-5). Further, these Fabry T-Rapa transduced cells were able to reduce substrate (Gb₃) in the Fabry mice, as depicted in FIG. 21. Substrate is reduced 4 weeks after transplant of transduced Fabry donor T-Rapa (n=4-5).

This Example demonstrates transduced T-Rapa express and secrete the enzymes necessary to counter lysosomal storage diseases (e.g., α-gal A and GCase). Further, this Example shows that transduced Fabry patient T-Rapa cells can be manufactured and transduced T-Rapa cells are able to function in vivo to reduce substrate.

### Example 5: Treatment of Fabry Disease

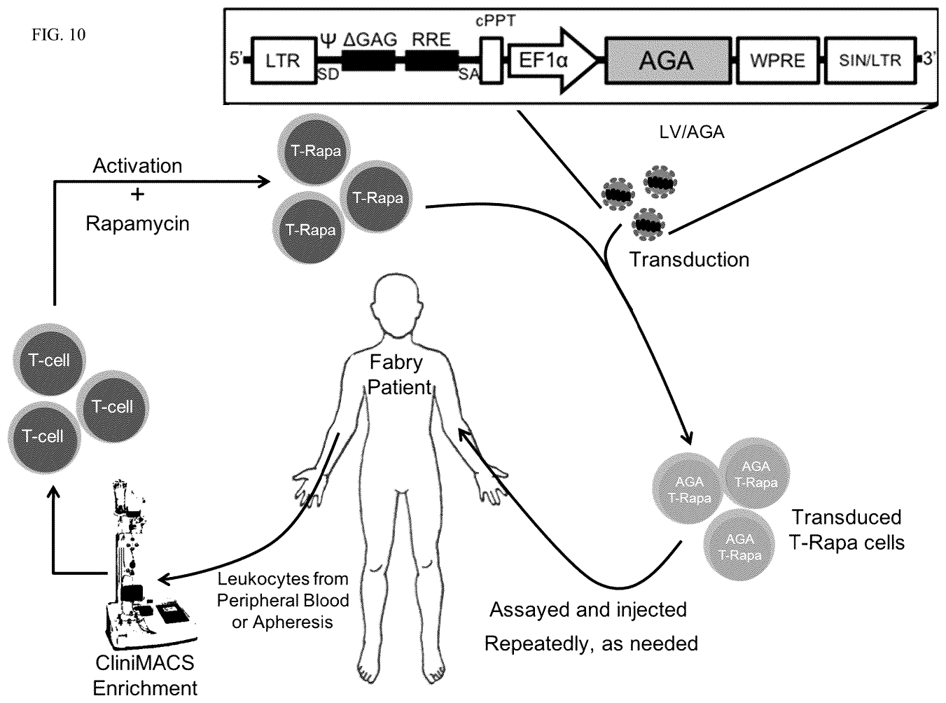

FIG. 10 depicts a schematic of the overall protocol for treatment of a disorder by the methods described herein, specifically showing the steps for treatment of a lysosomal storage disorder, for example, Fabry disease using transduced T-Rapa cells.

Patients' peripheral blood is collected by known methods in the art. CD4+ T-cells are then isolated from peripheral blood using methods known in the art, for example, flow cytometric cell sorting or magnetic cell sorting using antibodies against CD4. Alternatively, CD4+ cells can be similarly isolated from apheresis products, which may be obtained using methods known in the art. Isolated CD4+ cells are cultured in the presence of cytokines (IL-2 and IL-4) as described above in the presence of rapamycin (for example, 1 micromolar) for 3 days. The T-Rapa cells are transduced using a lentivirus ex vivo at an MOI of 1-30 or 1-60 for 12-18 hours, after which they are cultured in cytokine-containing medium for an additional 3 days (can be cultured from 3 days to about 1 month).

Patients receiving T-Rapa cells will not be conditioned with myelo-ablative chemotherapy. Rather, the types of chemotherapy to be administered will be lymphocyte-specific and myeloid-sparing. Lymphocyte-specific chemotherapy may consist of the following regimens (although other regimens can be envisioned): (1) fludarabine plus low-dose, daily cyclophosphamide; or (2) pentostatin plus low-dose daily cyclophosphamide.

Patients are infused with about 2-10×10⁶/kg transduced T-Rapa cells intravenously. Patients are monitored for expression of α-gal A. Cell administration may be repeated and cell dosage may be adjusted as recommended by appropriate physician.

### Example 6: Production of Dual Promoter Lentivirus Vectors for Use in the Present Methods

In some examples, the present invention may use dual promoter lentivirus vectors to transfer a transgene (e.g., AGA transgene) and a resistance gene (e.g., IMPDH2(IY)) to confer resistance to a drug (e.g., mycophenolate mofetil (MMF)) into the target T-cell. A dual promoter architecture (pDY-DP (SEQ ID NO:10)) was designed and constructed using pDY as a backbone using standard molecular biology techniques. Human-derived ubiquitous, constitutive promoters express transgenes of interest. For enrichment purposes, a vector with IMPDH2(IY) expressed from one promoter was constructed, with the ability to insert another transgene of interest from the other promoter (i.e. pDY−[MCS]+(IY), (SEQ ID NO:11)). A vector with AGA transgene was constructed to use in treating Fabry disease (SEQ ID NO:3). The titer for this vector is in the range of 1×10⁹ infectious viral particles (IVP)/mL. A vector with enhanced green fluorescent protein (eGFP) instead of IMPDH2(IY) was used to measure expression and for use as a non-enrichment control. FIG. 13 show the vector maps of lentiviral vectors used in the present invention.

Suitable methods of producing lentiviral vectors are known in the art. A suitable protocol is shown in FIG. 11. Production of lentivirus includes the following steps: (a) Three packaging plasmids, pCMVΔR8.91 (Zufferey, et al. Nature Biotechnology 15:871-874, 1997, incorporated by reference), pMD.G (Naldini et al., Science 272:263-267, 1996, incorporated by reference) and pAdV (Promega, USA) are mixed in appropriate ratios with a plasmid encoding the transfer vector of interest. These are complexed with polyethylenimine (PEI) and transfected into HEK293T cells. Media is replaced after 16 hours. (b) Culture supernatant containing virus particles is harvested approximately 40 and 64 hours after transfection. The supernatant is filtered through 0.22 μm membrane filters to remove contaminants. (c) Collected supernatant is subjected to ultracentrifugation as indicated to concentrate virus. (d) Viral pellets are resuspended in appropriate media in 2000-fold less volume than the original supernatant.

### Example 7: Exemplary Treatment of Fabry Disease Using Dual Promoter Lentiviral Vectors

Fabry patients will be treated as described in Example 5, with the exception that a dual promoter lentiviral vector, for example as described in Example 6 will be used in which the vector in addition to expressing α-gal A expresses IMPDH2(IY), which presents a growth advantage to transduced T-Rapa cells when the patient is treated with low doses of MMF.

Enrichment can be initiated if required by treatment with mycophenolate mofetil (MMF; CellCept, Roche, or approved generic); transduced T-Rapa cells are resistant to the effects of this drug, providing them with a growth advantage. A low dose of oral MMF may be effective (0.1-5 mg/kg TID) but higher doses (5-10 mg/kg TID or 1000 mg BID) may also be tolerated, depending on the patient. As a general guideline, a blood concentration of 0.4-2 μM free mycophenolic acid (MPA) is desirable. MMF may be administered for the duration for which increased enzyme activity is desired, and doses adjusted to titrate the activity. MMF may also be substituted for MPA formulations (Myfortic, Novartis, or approved generic).

Each publication, patent, and patent publication cited in this disclosure is incorporated in reference herein in its entirety. The present invention is not intended to be limited to the foregoing examples but encompasses all such modifications and variations as come within the scope of the appended claims.

The following sequences correspond with the plasmid maps in FIG. 13.

### Genetic Elements of Plasmids and Lentivirus Vectors

f1 ori: Origin of plasmid replication in bacteria
Amp(R): Ampicillin-resistance gene (beta-lactamase)
5′ LTR: HIV1-derived 5′ Long Terminal Repeat: Viral element required for integration into host genome
3′ LTR/SIN: HIV1-derived 3′ Long Terminal Repeat with a 133 bp deletion to inactivate the capacity for any viral replication after retrotranscription in the host cell
Psi sequence: Retroviral Psi packaging element
5′Gag (del3rdG): Viral element required in 2^{nd} generation lentivirus systems for proviral RNA transcription
RRE: Viral REV response element, required for nuclear export of proviral RNA
cPPT: central polypurine tract, a lentiviral element that enhances nuclear import/export of viral RNA, consequently enhancing viral titer and transduction.
EF1a: Ubiquitous, constitutively expressing promoter derived from the human elongation factor 1 alpha gene
WPRE: Woodchuck Hepatitis post-transcriptional regulatory element; potently terminates transcription and stabilizes mRNA.
hPGK: Ubiquitous, constitutively expressing promoter derived from the human phosphoglycerate kinase 1 gene
CTE and polyA: C-Terminal end and polyA signal sequence for termination of transcription
coIMPDH2(IY): codon optimized transgene expressing the T333I, S351Y mutant of human inosine-5′-monophosphate dehydrogenase 2
AGA/GBA/ASAH1/GAA: Codon optimized transgenes of interest encoding human α-galactosidase A, β-Glucocerebrosidase, N-acylsphingosine amidohydrolase 1, or acid α-glucosidase, respectively

### SEQUENCES

(AGA)
>co.hAGA
SEQ ID NO: 1
ATGCAACTTCGAAACCCAGAGCTCCACCTCGGATGTGCCCTTGCTCTGAGGTTCCTGGCGCTG
GTGTCTTGGGATATACCCGGAGCACGCGCTCTGGACAACGGGCTGGCCCGGACTCCAACCATG
GGTTGGCTCCATTGGGAAAGGTTTATGTGCAACTTGGACTGCCAGGAAGAACCCGACTCCTG
TATTTCCGAGAAACTCTTCATGGAGATGGCCGAGCTGATGGTTAGCGAAGGCTGGAAGGATG
CCGGTTATGAATACTTGTGTATCGACGATTGTTGGATGGCTCCCCAGCGGGACAGTGAAGGA
CGACTCCAGGCAGATCCGCAACGGTTCCCTCATGGCATACGGCAGCTCGCCAATTACGTGCAC
AGCAAGGGTTTGAAGCTGGGGATATATGCTGACGTGGGCAACAAAACCTGTGCTGGTTTCCC
CGGCAGCTTCGGCTACTATGATATAGATGCACAAACCTTCGCTGATTGGGGCGTGGACCTGCT
TAAATTTGACGGCTGTTACTGCGACAGCTTGGAAAACCTCGCCGATGGATATAAACACATGA
GCCTTGCACTCAATCGGACTGGCCGGAGCATTGTCTACTCTTGCGAGTGGCCATTGTACATGT
GGCCTTTCCAGAAGCCTAACTATACGGAGATTAGACAGTATTGTAATCACTGGAGAAACTTT
GCAGATATCGACGACTCATGGAAGTCCATCAAATCTATTCTGGACTGGACTTCATTCAATCA
GGAGCGCATCGTCGATGTTGCCGGTCCAGGTGGATGGAACGACCCTGACATGCTCGTAATTG
GGAATTTCGGACTGTCCTGGAATCAGCAGGTCACACAGATGGCTTTGTGGGCTATCATGGCA
GCCCCACTCTTTATGTCTAACGATTTGCGGCATATTTCACCACAGGCCAAAGCCCTGCTGCAA
GATAAGGACGTCATAGCGATTAACCAGGACCCACTGGGAAAGCAGGGCTACCAGCTGAGACA
GGGCGACAATTTTGAGGTCTGGGAAAGACCTCTTAGCGGGCTGGCGTGGGCCGTAGCCATGA
TTAATCGCCAGGAAATTGGCGGCCCTCGCTCTTACACTATCGCGGTCGCCAGTCTGGGCAAGG
GAGTCGCTTGTAACCCCGCCTGCTTCATAACTCAGTTGCTGCCCGTGAAACGGAAGCTGGGCT
TCTATGAATGGACTAGCAGACTCCGCAGTCATATTAATCCGACTGGTACGGTGCTGCTGCAA
CTGGAGAATACCATGCAGATGTCACTTAAGGATCTTCTGTGA
>pDY co.hAGA
SEQ ID NO: 2
AAATTGTAAGCGTTAATATTTTGTTAAAATTCGCGTTAAATTTTTGTTAAATCAGCTCATTT
TTTAACCAATAGGCCGAAATCGGCAAAATCCCTTATAAATCAAAAGAATAGACCGAGATAGG
GTTGAGTGTTGTTCCAGTTTGGAACAAGAGTCCACTATTAAAGAACGTGGACTCCAACGTCA
AAGGGCGAAAAACCGTCTATCAGGGCGATGGCCCACTACGTGAACCATCACCCTAATCAAGT
TTTTTGGGGTCGAGGTGCCGTAAAGCACTAAATCGGAACCCTAAAGGGAGCCCCCGATTTAG
AGCTTGACGGGGAAAGCCGGCGAACGTGGCGAGAAAGGAAGGGAAGAAAGCGAAAGGAGCG
GGCGCTAGGGCGCTGGCAAGTGTAGCGGTCACGCTGCGCGTAACCACCACACCCGCCGCGCTT
AATGCGCCGCTACAGGGCGCGTCCATTCGCCATTCAGGCTGCGCAACTGTTGGGAAGGGCGAT
CGGTGCGGGCCTCTTCGCTATTACGCCAGCTGGCGAAAGGGGGATGTGCTGCAAGGCGATTA
AGTTGGGTAACGCCAGGGTTTTCCCAGTCACGACGTTGTAAAACGACGGCCAGTGAATTGTA
ATACGACTCACTATAGGGCGAATTGGGCCCGACGTCGCATGCTTGGAAGGGCTAATTCACTCC
CAAAGAAGACAAGATATCCTTGATCTGTGGATCTACCACACACAAGGCTACTTCCCTGATTA
GCAGAACTACACACCAGGGCCAGGGGTCAGATATCCACTGACCTTTGGATGGTGCTACAAGC
TAGTACCAGTTGAGCCAGATAAGGTAGAAGAGGCCAATAAAGGAGAGAACACCAGCTTGTTA
CACCCTGTGAGCCTGCATGGGATGGATGACCCGGAGAGAGAAGTGTTAGAGTGGAGGTTTGA
CAGCCGCCTAGCATTTCATCACGTGGCCCGAGAGCTGCATCCGGAGTACTTCAAGAACTGCTG
ATATCGAGCTTGCTACAAGGGACTTTCCGCTGGGGACTTTCCAGGGAGGCGTGGCCTGGGCG
GGACTGGGGAGTGGCGAGCCCTCAGATCCTGCATATAAGCAGCTGCTTTTTGCCTGTACTGGG
TCTCTCTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGACTCT
GGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGC
TGAAGCGCGCACGGCAAGAGGCGAGGGGCGGCGACTGGTGAGTACGCCAAAAATTTTGACTA
GCGGAGGCTAGAAGGAGAGAGATGGGTGCGAGAGCGTCAGTATTAAGCGGGGGAGAATTAG
ATCGCGATGGGAAAAAATTCGGTTAAGGCCAGGGGGAAAGAAAAAATATAAATTAAAACAT
ATAGTATGGGCAAGCAGGGAGCTAGAACGATTCGCAGTTAATCCTGGCCTGTTAGAAACATC
AGAAGGCTGTAGACAAATACTGGGACAGCTACAACCATCCCTTCAGACAGGATCAGAAGAAC
TTAGATCATTATATAATACAGTAGCAACCCTCTATTGTGTGCATCAAAGGATAGAGATAAAA
GACACCAAGGAAGCTTTAGACAAGATAGAGGAAGAGCAAAACAAAAGTAAGACCACCGCACA
GCAAGCGGCCGCTGATCTTCAGACCTGGAGGAGGAGATATGAGGGACAATTGGAGAAGTGAA
TTATATAAATATAAAGTAGTAAAAATTGAACCATTAGGAGTAGCACCCACCAAGGCAAAGAG
AAGAGTGGTGCAGAGAGAAAAAAGAGCAGTGGGAATAGGAGCTTTGTTCCTTGGGTTCTTGG
GAGCAGCAGGAAGCACTATGGGCGCAGCGTCAATGACGCTGACGGTACAGGCCAGACAATTA
TTGTCTGGTATAGTGCAGCAGCAGAACAATTTGCTGAGGGCTATTGAGGCGCAACAGCATCT
GTTGCAACTCACAGTCTGGGGCATCAAGCAGCTCCAGGCAAGAATCCTGGCTGTGGAAAGAT
ACCTAAAGGATCAACAGCTCCTGGGGATTTGGGGTTGCTCTGGAAAACTCATTTGCACCACT
GCTGTGCCTTGGAATGCTAGTTGGAGTAATAAATCTCTGGAACAGATTTGGAATCACACGAC
CTGGATGGAGTGGGACAGAGAAATTAACAATTACACAAGCTTAATACACTCCTTAATTGAAG
AATCGCAAAACCAGCAAGAAAAGAATGAACAAGAATTATTGGAATTAGATAAATGGGCAAG
TTTGTGGAATTGGTTTAACATAACAAATTGGCTGTGGTATATAAAATTATTCATAATGATAG
TAGGAGGCTTGGTAGGTTTAAGAATAGTTTTTGCTGTACTTTCTATAGTGAATAGAGTTAGG
CAGGGATATTCACCATTATCGTTTCAGACCCACCTCCCAACCCCGAGGGGACCCGACAGGCCC
GAAGGAATAGAAGAAGAAGGTGGAGAGAGAGACAGAGACAGATCCATTCGATTAGTGAACG
GATCTCGACGGGATCGATTTTAAAAGAAAAGGGGGGATTGGGGGGTACAGTGCAGGGGAAA
GAATAGTAGACATAATAGCAACAGACATACAAACTAAAGAATTACAAAAACAAATTACAAA
AATTCAAAATTTTATCGATAAGCTTTGCAAAGATGGATAAAGTTTTAAACAGAGAGGAATCT
TTGCAGCTAATGGACCTTCTAGGTCTTGAAAGGAGTGGGAATTGGCTCCGGTGCCCGTCAGT
GGGCAGAGCGCACATCGCCCACAGTCCCCGAGAAGTTGGGGGGAGGGGTCGGCAATTGAACC
GGTGCCTAGAGAAGGTGGCGCGGGGTAAACTGGGAAAGTGATGTCGTGTACTGGCTCCGCCT
TTTTCCCGAGGGTGGGGGAGAACCGTATATAAGTGCAGTAGTCGCCGTGAACGTTCTTTTTC
GCAACGGGTTTGCCGCCAGAACACAGGTAAGTGCCGTGTGTGGTTCCCGCGGGCCTGGCCTCT
TTACGGGTTATGGCCCTTGCGTGCCTTGAATTACTTCCACCTGGCTGCAGTACGTGATTCTTG
ATCCCGAGCTTCGGGTTGGAAGTGGGTGGGAGAGTTCGAGGCCTTGCGCTTAAGGAGCCCCT
TCGCCTCGTGCTTGAGTTGAGGCCTGGCCTGGGCGCTGGGGCCGCCGCGTGCGAATCTGGTGG
CACCTTCGCGCCTGTCTCGCTGCTTTCGATAAGTCTCTAGCCATTTAAAATTTTTGATGACCT
GCTGCGACGCTTTTTTTCTGGCAAGATAGTCTTGTAAATGCGGGCCAAGATCTGCACACTGG
TATTTCGGTTTTTGGGGCCGCGGGCGGCGACGGGGCCCGTGCGTCCCAGCGCACATGTTCGGC
GAGGCGGGGCCTGCGAGCGCGGCCACCGAGAATCGGACGGGGGTAGTCTCAAGCTGGCCGGCC
TGCTCTGGTGCCTGGCCTCGCGCCGCCGTGTATCGCCCCGCCCTGGGCGGCAAGGCTGGCCCG
GTCGGCACCAGTTGCGTGAGCGGAAAGATGGCCGCTTCCCGGCCCTGCTGCAGGGAGCTCAAA
ATGGAGGACGCGGCGCTCGGGAGAGCGGGCGGGTGAGTCACCCACACAAAGGAAAAGGGCCT
TTCCGTCCTCAGCCGTCGCTTCATGTGACTCCACGGAGTACCGGGCGCCGTCCAGGCACCTCG
ATTAGTTCTCGAGCTTTTGGAGTACGTCGTCTTTAGGTTGGGGGGAGGGGTTTTATGCGATG
GAGTTTCCCCACACTGAGTGGGTGGAGACTGAAGTTAGGCCAGCTTGGCACTTGATGTAATT
CTCCTTGGAATTTGCCCTTTTTGAGTTTGGATCTTGGTTCATTCTCAAGCCTCAGACAGTGGT
TCAAAGTTTTTTTCTTCCATTTCAGGTGTCGTGAGGAATTCGCCACCATGCAACTTCGAAACC
CAGAGCTCCACCTCGGATGTGCCCTTGCTCTGAGGTTCCTGGCGCTGGTGTCTTGGGATATAC
CCGGAGCACGCGCTCTGGACAACGGGCTGGCCCGGACTCCAACCATGGGTTGGCTCCATTGGG
AAAGGTTTATGTGCAACTTGGACTGCCAGGAAGAACCCGACTCCTGTATTTCCGAGAAACTC
TTCATGGAGATGGCCGAGCTGATGGTTAGCGAAGGCTGGAAGGATGCCGGTTATGAATACTT
GTGTATCGACGATTGTTGGATGGCTCCCCAGCGGGACAGTGAAGGACGACTCCAGGCAGATC
CGCAACGGTTCCCTCATGGCATACGGCAGCTCGCCAATTACGTGCACAGCAAGGGTTTGAAGC
TGGGGATATATGCTGACGTGGGCAACAAAACCTGTGCTGGTTTCCCCGGCAGCTTCGGCTACT
ATGATATAGATGCACAAACCTTCGCTGATTGGGGCGTGGACCTGCTTAAATTTGACGGCTGT
TACTGCGACAGCTTGGAAAACCTCGCCGATGGATATAAACACATGAGCCTTGCACTCAATCG
GACTGGCCGGAGCATTGTCTACTCTTGCGAGTGGCCATTGTACATGTGGCCTTTCCAGAAGCC
TAACTATACGGAGATTAGACAGTATTGTAATCACTGGAGAAACTTTGCAGATATCGACGACT
CATGGAAGTCCATCAAATCTATTCTGGACTGGACTTCATTCAATCAGGAGCGCATCGTCGAT
GTTGCCGGTCCAGGTGGATGGAACGACCCTGACATGCTCGTAATTGGGAATTTCGGACTGTC
CTGGAATCAGCAGGTCACACAGATGGCTTTGTGGGCTATCATGGCAGCCCCACTCTTTATGTC
TAACGATTTGCGGCATATTTCACCACAGGCCAAAGCCCTGCTGCAAGATAAGGACGTCATAG
CGATTAACCAGGACCCACTGGGAAAGCAGGGCTACCAGCTGAGACAGGGCGACAATTTTGAG
GTCTGGGAAAGACCTCTTAGCGGGCTGGCGTGGGCCGTAGCCATGATTAATCGCCAGGAAAT
TGGCGGCCCTCGCTCTTACACTATCGCGGTCGCCAGTCTGGGCAAGGGAGTCGCTTGTAACCC
CGCCTGCTTCATAACTCAGTTGCTGCCCGTGAAACGGAAGCTGGGCTTCTATGAATGGACTAG
CAGACTCCGCAGTCATATTAATCCGACTGGTACGGTGCTGCTGCAACTGGAGAATACCATGC
AGATGTCACTTAAGGATCTTCTGTGAGAACCCGGGATCCAAGCTTCAATTGTGGTCACTCGA
CAATCAACCTCTGGATTACAAAATTTGTGAAAGATTGACTGGTATTCTTAACTATGTTGCTC
CTTTTACGCTATGTGGATACGCTGCTTTAATGCCTTTGTATCATGCTATTGCTTCCCGTATGG
CTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATTC
CGTGGTGTTGTCGGGGAAGCTGACGTCCTTTCCATGGCTGCTCGCCTGTGTTGCCACCTGGAT
TCTGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGATCT
CCCTTTGGGCCGCCTCCCCGCCTGCTCGAGACCTAGAAAAACATGGAGCAATCACAAGTAGCA
ATACAGCAGCTACCAATGCTGATTGTGCCTGGCTAGAAGCACAAGAGGAGGAGGAGGTGGGT
TTTCCAGTCACACCTCAGGTACCTTTAAGACCAATGACTTACAAGGCAGCTGTAGATCTTAGC
CACTTTTTAAAAGAAAAGGGGGGACTGGAAGGGCTAATTCACTCCCAACGAAGACAAGATCT
GCTTTTTGCTTGTACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTAGTGTGTG
CCCGTCTGTTGTGTGACTCTGGTAACTAGAGATCCCTCAGACCCTTTTAGTCAGTGTGGAAAA
TCTCTAGCAGTAGTAGTTCATGTCATCTTATTATTCAGTATTTATAACTTGCAAAGAAATGA
ATATCAGAGAGTGAGAGGACGCGTTGGATGCATAGCTTGAGTATTCTATAGTGTCACCTAAA
TAGCTTGGCGTAATCATGGTCATAGCTGTTTCCTGTGTGAAATTGTTATCCGCTCACAATTCC
ACACAACATACGAGCCGGAAGCATAAAGTGTAAAGCCTGGGGTGCCTAATGAGTGAGCTAAC
TCACATTAATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCGCTCTTCCGCTTCCT
CGCTCACTGACTCGCTGCGCTCGGTCGTTCGGCTGCGGCGAGCGGTATCAGCTCACTCAAAGG
CGGTAATACGGTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATAGGCTCCGCCCC
CCTGACGAGCATCACAAAAATCGACGCTCAAGTCAGAGGTGGCGAAACCCGACAGGACTATA
AAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAAT
GCTTAATCAGTGAGGCACCTATCTCAGCGATCTGTCTATTTCGTTCATCCATAGTTGCCTGAC
TCCCCGTCGTGTAGATAACTACGATACGGGAGGGCTTACCATCTGGCCCCAGTGCTGCAATGA
TACCGCGAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATACCGCGCC
ACATAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCTTCAG
CATCTTTTACTTTCACCAGCGTTTCTGGGTGAGCAAAAACAGGAAGGCAAAATGCCGCAAAA
AAGGGAATAAGGGCGACACGGAAATGTTGAATACTCATACTCTTCCTTTTTCAATATTATTG
AAGCATTTATCAGGGTTATTGTCTCATGAGCGGATACATATTTGAATGTATTTAGAAAAATA
AACAAATAGGGGTTCCGCGCACATTTCCCCGAAAAGTGCCACCTGATGCGGTGTGAAATACC
GCACAGATGCGTAAGGAGAAAATACCGCATCAGG
>pDY AGA + (IY) (SEQ ID NO: 3)
SEQ ID NO: 3
AAATTGTAAGCGTTAATATTTTGTTAAAATTCGCGTTAAATTTTTGTTAAATCAGCTCATTT
TTTAACCAATAGGCCGAAATCGGCAAAATCCCTTATAAATCAAAAGAATAGACCGAGATAGG
GTTGAGTGTTGTTCCAGTTTGGAACAAGAGTCCACTATTAAAGAACGTGGACTCCAACGTCA
AAGGGCGAAAAACCGTCTATCAGGGCGATGGCCCACTACGTGAACCATCACCCTAATCAAGT
TTTTTGGGGTCGAGGTGCCGTAAAGCACTAAATCGGAACCCTAAAGGGAGCCCCCGATTTAG
AGCTTGACGGGGAAAGCCGGCGAACGTGGCGAGAAAGGAAGGGAAGAAAGCGAAAGGAGCG
GGCGCTAGGGCGCTGGCAAGTGTAGCGGTCACGCTGCGCGTAACCACCACACCCGCCGCGCTT
AATGCGCCGCTACAGGGCGCGTCCATTCGCCATTCAGGCTGCGCAACTGTTGGGAAGGGCGAT
CGGTGCGGGCCTCTTCGCTATTACGCCAGCTGGCGAAAGGGGGATGTGCTGCAAGGCGATTA
AGTTGGGTAACGCCAGGGTTTTCCCAGTCACGACGTTGTAAAACGACGGCCAGTGAATTGTA
ATACGACTCACTATAGGGCGAATTGGGCCCGACGTCGCATGCTTGGAAGGGCTAATTCACTCC
CAAAGAAGACAAGATATCCTTGATCTGTGGATCTACCACACACAAGGCTACTTCCCTGATTA
GCAGAACTACACACCAGGGCCAGGGGTCAGATATCCACTGACCTTTGGATGGTGCTACAAGC
TAGTACCAGTTGAGCCAGATAAGGTAGAAGAGGCCAATAAAGGAGAGAACACCAGCTTGTTA
CACCCTGTGAGCCTGCATGGGATGGATGACCCGGAGAGAGAAGTGTTAGAGTGGAGGTTTGA
CAGCCGCCTAGCATTTCATCACGTGGCCCGAGAGCTGCATCCGGAGTACTTCAAGAACTGCTG
ATATCGAGCTTGCTACAAGGGACTTTCCGCTGGGGACTTTCCAGGGAGGCGTGGCCTGGGCG
GGACTGGGGAGTGGCGAGCCCTCAGATCCTGCATATAAGCAGCTGCTTTTTGCCTGTACTGGG
TCTCTCTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGACTCT
GGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGC
TGAAGCGCGCACGGCAAGAGGCGAGGGGCGGCGACTGGTGAGTACGCCAAAAATTTTGACTA
GCGGAGGCTAGAAGGAGAGAGATGGGTGCGAGAGCGTCAGTATTAAGCGGGGGAGAATTAG
ATCGCGATGGGAAAAAATTCGGTTAAGGCCAGGGGGAAAGAAAAAATATAAATTAAAACAT
ATAGTATGGGCAAGCAGGGAGCTAGAACGATTCGCAGTTAATCCTGGCCTGTTAGAAACATC
AGAAGGCTGTAGACAAATACTGGGACAGCTACAACCATCCCTTCAGACAGGATCAGAAGAAC
TTAGATCATTATATAATACAGTAGCAACCCTCTATTGTGTGCATCAAAGGATAGAGATAAAA
GACACCAAGGAAGCTTTAGACAAGATAGAGGAAGAGCAAAACAAAAGTAAGACCACCGCACA
GCAAGCGGCCGCTGATCTTCAGACCTGGAGGAGGAGATATGAGGGACAATTGGAGAAGTGAA
TTATATAAATATAAAGTAGTAAAAATTGAACCATTAGGAGTAGCACCCACCAAGGCAAAGAG
AAGAGTGGTGCAGAGAGAAAAAAGAGCAGTGGGAATAGGAGCTTTGTTCCTTGGGTTCTTGG
GAGCAGCAGGAAGCACTATGGGCGCAGCGTCAATGACGCTGACGGTACAGGCCAGACAATTA
TTGTCTGGTATAGTGCAGCAGCAGAACAATTTGCTGAGGGCTATTGAGGCGCAACAGCATCT
GTTGCAACTCACAGTCTGGGGCATCAAGCAGCTCCAGGCAAGAATCCTGGCTGTGGAAAGAT
ACCTAAAGGATCAACAGCTCCTGGGGATTTGGGGTTGCTCTGGAAAACTCATTTGCACCACT
GCTGTGCCTTGGAATGCTAGTTGGAGTAATAAATCTCTGGAACAGATTTGGAATCACACGAC
CTGGATGGAGTGGGACAGAGAAATTAACAATTACACAAGCTTAATACACTCCTTAATTGAAG
AATCGCAAAACCAGCAAGAAAAGAATGAACAAGAATTATTGGAATTAGATAAATGGGCAAG
TTTGTGGAATTGGTTTAACATAACAAATTGGCTGTGGTATATAAAATTATTCATAATGATAG
TAGGAGGCTTGGTAGGTTTAAGAATAGTTTTTGCTGTACTTTCTATAGTGAATAGAGTTAGG
CAGGGATATTCACCATTATCGTTTCAGACCCACCTCCCAACCCCGAGGGGACCCGACAGGCCC
GAAGGAATAGAAGAAGAAGGTGGAGAGAGAGACAGAGACAGATCCATTCGATTAGTGAACG
GATCTCGACGGGATCGATTTTAAAAGAAAAGGGGGGATTGGGGGGTACAGTGCAGGGGAAA
GAATAGTAGACATAATAGCAACAGACATACAAACTAAAGAATTACAAAAACAAATTACAAA
AATTCAAAATTTTATCGATAAGCTTTGCAAAGATGGATAAAGTTTTAAACAGAGAGGAATCT
TTGCAGCTAATGGACCTTCTAGGTCTGACCCCGTACGCCTCGAGAGATCTGATCATAATCAGC
CATACCACATTTGTAGAGGTTTTACTTGCTTTAAAAAACCTCCCACACCTCCCCCTGAACCTG
AAACATAAAATGAATGCAATTGTTGTTGTTAACTTGTTTATTGCAGCTTATAATGGTTACAA
ATAAGGCAATAGCATCACAAATTTCACAAATAAGGCATTTTTTTCACTGCATTCTAGTTTTG
GTTTGTCCAAACTCATCAATGTATCTTATCATGTCTGGATCTCAAATCCCTCGGAAGCTGCGC
CTGTCTTAGGTTGGAGTGATACATTTTTATCACTTTTACCCGTCTTTGGATTAGGCAGTAGC
TCTGACGGCCCTCCTGTCTTAGGTTAGTGAAAAATGTCACTCTCTTACCCGTCATTGGCTGTC
CAGCTTAGCTCGCAGGGGAGGTGGTCTGCCTGCAGGTTAGAACAGTCTCTTTTCGTATGAGT
GCAGTGAGTGGACGCCGCCTTCGACCTGGGCTGAAGAAGTTCTTTTCTCGAACTTCAGTTCGC
CGGAATACATCATTGCCCGCACCTGTGTCAGGCTCTTAGCGCCGATATCCTGGCATGAATGCT
GAATTCCGGCGATCAGGTAAGGCACGAATTTGTGAATACTGCCCTTATCCTGGACAGCTCCA
GACACGCCCTGTGCGACTTTGATCTTGTCTGCCTCGGAAAAATACCTGTTCTGAGAGGACAGA
TGCTTATCCATGGCGTCCAGTGACCCCATGCCCCTATATTTCTTCAGTCTGAACCCATCACTA
AAGAAGTACTCGCCGGGGGCTTCTGTGGTTGCAGCCAGCAGGCTGCCCATCATCACTGTGCTT
GCCCCCAGAGCCAGGGCTTTTGCGATGTGGCCCACATTCTGAATTCCCCCGTCAGCGATCACT
GGGACTCCGAATCTCCGGGCATACTCGTACACCTTGTAGACAGCAGTTGCCTGAGGTCGTCCA
CAGGCCAGCACTTCCTGAATGATGCAGATTGATCCACTCCCCATTCCGACCCTCAGAGCATCC
ACTCCTGCGTCAATCAGGTTTTTGGCCTGGGCTGCGGTCACGACATTGCCTCCGATGACCTGC
AGATTTGGGTACTTGTCCTTAATGTACTTGATCATATTAATCTGGAAGATGCTGTTTCCCTG
GCTTGAATCCAGCACGACCACGTCCACCCCTGCCTGAGCCAGCAGATCCAGGCGATATTTATC
GTCCTCGTGTGTGCCAATAGCGGCTCCACACAGCAGCTGTTTCTTTGCGTCCTTACTAGCCAG
AGGGTAATCTCGATTTTTCTTCAGGTCGGTGCGGGCAATGATTGCCACCAGCTCATCGTCTTC
ATTCACGATAGGCAGTTTTCCTTTCTTAGACCGCTGCAGAATCTCGTTGGCTTCCTTCAGTGT
GATGCCGGCAGGTGCGACCACCAGATCTTCGCGTTTGGTCATAATCTCTTCCAGAAAACAGTC
ATGCTCTTCCTCCTTCAGGAAATCGATGTCTCGACTAGAAATGATTCCCACCAGTCGGCTGCC
CATTCGTCCAGTATCTGTAATGGGGATGCCGCAAAATCCGTGCCTAGCTTTGGCCTCGAACAC
ATCGCGGACCCTGTCCTTGGGGCTCAGGACCACTGGGTCGGTGATAAAGCCCTGTTCGTATTT
CTTCACCTTTCTGACCTCATTGGCCTGAAATTCTGGAGTGCAGTTATGGTGAATGAACCCGAT
CCCGCCTGTCAGTGCCATAGCAATGGCCATGCCAGCCTCGGTGACAGTGTCCATAGGGGAGCT
CACCAGGGGTGTCTTCAGGGTGATTTTCTTGGTCAGGGCAGAAGTCAGATCCACCTGGTCTGC
GGTAAAATCAATATAGCCGGGCAGGATCAGGAAGTCGTTGTAAGTCAGCCCGTCTCCACAAT
TAAACAGCTGCTGGGCGGTCAGTCCATCATCAGGGACATAGGAAGTGCCTCCAGAAATCAGG
TAGTCGGCCATGGTGGCGCTAGCCCTGGGGAGAGAGGTCGGTGATTCGGTCAACGAGGGAGC
CGACTGCCGACGTGCGCTCCGGAGGCTTGCAGAATGCGGAACACCGCGCGGGCAGGAACAGG
GCCCACACTACCGCCCCACACCCCGCCTCCCGCACCGCCCCTTCCCGGCCGCTGCTCTCGGCGC
GCCCCGCTGAGCAGCCGCTATTGGCCACAGCCCATCGCGGTCGGCGCGCTGCCATTGCTCCCT
GGCGCTGTCCGTCTGCGAGGGTACTAGTGAGACGTGCGGCTTCCGTTTGTCACGTCCGGCACG
CCGCGAACCGCAAGGAACCTTCCCGACTTAGGGGCGGAGCAGGAAGCGTCGCCGGGGGGCCCA
CAAGGGTAGCGGCGAAGATCCGGGTGACGCTGCGAACGGACGTGAAGAATGTGCGAGACCCA
GGGTCGGCGCCGCTGCGTTTCCCGGAACCACGCCCAGAGCAGCCGCGTCCCTGCGCAAACCCA
GGGCTGCCAAGGAAAAGGCGCAACCCCAACCCCGTGGTTAATTAAGGTGAAAGGAGTGGGAA
TTGGCTCCGGTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGGGAAAGTGAT
GTCGTGTACTGGCTCCGCCTTTTTCCCGAGGGTGGGGGAGAACCGTATATAAGTGCAGTAGT
CGCCGTGAACGTTCTTTTTCGCAACGGGTTTGCCGCCAGAACACAGGTAAGTGCCGTGTGTGG
TTCCCGCGGGCCTGGCCTCTTTACGGGTTATGGCCCTTGCGTGCCTTGAATTACTTCCACCTG
GCTGCAGTACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCGCTGGGGCC
GCCGCGTGCGAATCTGGTGGCACCTTCGCGCCTGTCTCGCTGCTTTCGATAAGTCTCTAGCCA
TTTAAAATTTTTGATGACCTGCTGCGACGCTTTTTTTCTGGCAAGATAGTCTTGTAAATGCG
GGCCAAGATCTGCACACTGGTATTTCGGTTTTTGGGGCCGCGGGCGGCGACGGGGCCCGTGCG
TCCCAGCGCACATGTTCGGCGAGGCGGGGCCTGCGAGCGCGGCCACCGAGAATCGGACGGGGG
TAGTCTCAAGCTGGCCGGCCTGCTCTGGTGCCTGGCCTCGCGCCGCCGTGTATCGCCCCGCCCT
GGGCGGCAAGGCTGGCCCGGTCGGCACCAGTTGCGTGAGCGGAAAGATGGCCGCTTCCCGGCC
CTGCTGCAGGGAGCTCAAAATGGAGGACGCGGCGCTCGGGAGAGCGGGCGGGTGAGTCACCC
ACACAAAGGAAAAGGGCCTTTCCGTCCTCAGCCGTCGCTTCATGTGACTCCACGGAGTACCGG
GCGCCGTCCAGGCACCTCGATTAGTTCTCGAGCTTTTGGAGTACGTCGTCTTTAGGTTGGGGG
GAGGGGTTTTATGCGATGGAGTTTCCCCACACTGAGTGGGTGGAGACTGAAGTTAGGCCAGC
TTGGCACTTGATGTAATTCTCCTTGGAATTTGCCCTTTTTGAGTTTGGATCTTGGTTCATTCT
CAAGCCTCAGACAGTGGTTCAAAGTTTTTTTCTTCCATTTCAGGTGTCGTGAGGAATTCGCCA
CCATGCAACTTCGAAACCCAGAGCTCCACCTCGGATGTGCCCTTGCTCTGAGGTTCCTGGCGC
TGGTGTCTTGGGATATACCCGGAGCACGCGCTCTGGACAACGGGCTGGCCCGGACTCCAACCA
TGGGTTGGCTCCATTGGGAAAGGTTTATGTGCAACTTGGACTGCCAGGAAGAACCCGACTCC
TGTATTTCCGAGAAACTCTTCATGGAGATGGCCGAGCTGATGGTTAGCGAAGGCTGGAAGGA
TGCCGGTTATGAATACTTGTGTATCGACGATTGTTGGATGGCTCCCCAGCGGGACAGTGAAG
GACGACTCCAGGCAGATCCGCAACGGTTCCCTCATGGCATACGGCAGCTCGCCAATTACGTGC
ACAGCAAGGGTTTGAAGCTGGGGATATATGCTGACGTGGGCAACAAAACCTGTGCTGGTTTC
CCCGGCAGCTTCGGCTACTATGATATAGATGCACAAACCTTCGCTGATTGGGGCGTGGACCTG
CTTAAATTTGACGGCTGTTACTGCGACAGCTTGGAAAACCTCGCCGATGGATATAAACACAT
GAGCCTTGCACTCAATCGGACTGGCCGGAGCATTGTCTACTCTTGCGAGTGGCCATTGTACAT
GTGGCCTTTCCAGAAGCCTAACTATACGGAGATTAGACAGTATTGTAATCACTGGAGAAACT
TTGCAGATATCGACGACTCATGGAAGTCCATCAAATCTATTCTGGACTGGACTTCATTCAAT
CAGGAGCGCATCGTCGATGTTGCCGGTCCAGGTGGATGGAACGACCCTGACATGCTCGTAAT
TGGGAATTTCGGACTGTCCTGGAATCAGCAGGTCACACAGATGGCTTTGTGGGCTATCATGG
CAGCCCCACTCTTTATGTCTAACGATTTGCGGCATATTTCACCACAGGCCAAAGCCCTGCTGC
AAGATAAGGACGTCATAGCGATTAACCAGGACCCACTGGGAAAGCAGGGCTACCAGCTGAGA
CAGGGCGACAATTTTGAGGTCTGGGAAAGACCTCTTAGCGGGCTGGCGTGGGCCGTAGCCAT
GATTAATCGCCAGGAAATTGGCGGCCCTCGCTCTTACACTATCGCGGTCGCCAGTCTGGGCAA
GGGAGTCGCTTGTAACCCCGCCTGCTTCATAACTCAGTTGCTGCCCGTGAAACGGAAGCTGGG
CTTCTATGAATGGACTAGCAGACTCCGCAGTCATATTAATCCGACTGGTACGGTGCTGCTGCA
ACTGGAGAATACCATGCAGATGTCACTTAAGGATCTTCTGTGAGAACCCGGGATCCAAGCTT
CAATTGTGGTCACTCGACAATCAACCTCTGGATTACAAAATTTGTGAAAGATTGACTGGTAT
TCTTAACTATGTTGCTCCTTTTACGCTATGTGGATACGCTGCTTTAATGCCTTTGTATCATGC
TATTGCTTCCCGTATGGCTTTCATTTTCTCCTCCTTGTATAAATCCTGGTTGCTGTCTCTTTA
TGAGGAGTTGTGGCCCGTTGTCAGGCAACGTGGCGTGGTGTGCACTGTGTTTGCTGACGCAA
CCCCCACTGGTTGGGGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGACAGGGGCTCGGCT
GTTGGGCACTGACAATTCCGTGGTGTTGTCGGGGAAGCTGACGTCCTTTCCATGGCTGCTCGC
CTGTGTTGCCACCTGGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCGCGTCTTCGCCTTCGC
CCTCAGACGAGTCGGATCTCCCTTTGGGCCGCCTCCCCGCCTGCTCGAGACCTAGAAAAACAT
GGAGCAATCACAAGTAGCAATACAGCAGCTACCAATGCTGATTGTGCCTGGCTAGAAGCACA
AGAGGAGGAGGAGGTGGGTTTTCCAGTCACACCTCAGGTACCTTTAAGACCAATGACTTACA
AGGCAGCTGTAGATCTTAGCCACTTTTTAAAAGAAAAGGGGGGACTGGAAGGGCTAATTCAC
TCCCAACGAAGACAAGATCTGCTTTTTGCTTGTACTGGGTCTCTCTGGTTAGACCAGATCTGA
GCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCC
TTTTAGTCAGTGTGGAAAATCTCTAGCAGTAGTAGTTCATGTCATCTTATTATTCAGTATTT
ATAACTTGCAAAGAAATGAATATCAGAGAGTGAGAGGACGCGTTGGATGCATAGCTTGAGTA
TTCTATAGTGTCACCTAAATAGCTTGGCGTAATCATGGTCATAGCTGTTTCCTGTGTGAAAT
TGTTATCCGCTCACAATTCCACACAACATACGAGCCGGAAGCATAAAGTGTAAAGCCTGGGG
TGCCTAATGAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGGCGGTTTGCGTA
TTGGGCGCTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATAACGCAGGA
AAGAACATGTGAGCAAAAGGCCAGCAAAAGGCCAGGAACCGTAAAAAGGCCGCGTTGCTGGC
GTTTTTCCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAG
AGCGAGGTATGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGCAGCA
GATTACGCGCAGAAAAAAAGGATCTCAAGAAGATCCTTTGATCTTTTCTACGGGGTCTGACG
CTCAGTGGAACGAAAACTCACGTTAAGGGATTTTGGTCATGAGATTATCAAAAAGGATCTTC
ACCTAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCATC
TGGCCCCAGTGCTGCAATGATACCGCGAGACCCACGCTCACCGGCTCCAGATTTATCAGCAAT
AAACCAGCCAGCCGGAAGGGCCGAGCGCAGAAGTGGTCCTGCAACTTTATCCGCCTCCATCCA
GTCTATTAATTGTTGCCGGGAAGCTAGAGTAAGTAGTTCGCCAGTTAATAGTTTGCGCAACG
TTGTTGCCATTGCTACAGGCATCGTGGTGTCACGCTCGTCGTTTGGTATGGCTTCATTCAGCT
CCGGTTCCCAACGATCAAGGCGAGTTACATGATCCCCCATGTTGTGCAAAAAAGCGGTTAGC
TCCTTCGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTCA
ATACGGGATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACC
TGATGCGGTGTGAAATACCGCACAGATGCGTAAGGAGAAAATACCGCATCAGG
Gaucher disease
>pDY co hGBA
SEQ ID NO: 4
AAATTGTAAGCGTTAATATTTTGTTAAAATTCGCGTTAAATTTTTGTTAAATCAGCTCATTT
TTTAACCAATAGGCCGAAATCGGCAAAATCCCTTATAAATCAAAAGAATAGACCGAGATAGG
GTTGAGTGTTGTTCCAGTTTGGAACAAGAGTCCACTATTAAAGAACGTGGACTCCAACGTCA
AAGGGCGAAAAACCGTCTATCAGGGCGATGGCCCACTACGTGAACCATCACCCTAATCAAGT
TTTTTGGGGTCGAGGTGCCGTAAAGCACTAAATCGGAACCCTAAAGGGAGCCCCCGATTTAG
AGCTTGACGGGGAAAGCCGGCGAACGTGGCGAGAAAGGAAGGGAAGAAAGCGAAAGGAGCG
GGCGCTAGGGCGCTGGCAAGTGTAGCGGTCACGCTGCGCGTAACCACCACACCCGCCGCGCTT
AATGCGCCGCTACAGGGCGCGTCCATTCGCCATTCAGGCTGCGCAACTGTTGGGAAGGGCGAT
CGGTGCGGGCCTCTTCGCTATTACGCCAGCTGGCGAAAGGGGGATGTGCTGCAAGGCGATTA
AGTTGGGTAACGCCAGGGTTTTCCCAGTCACGACGTTGTAAAACGACGGCCAGTGAATTGTA
ATACGACTCACTATAGGGCGAATTGGGCCCGACGTCGCATGCTTGGAAGGGCTAATTCACTCC
CAAAGAAGACAAGATATCCTTGATCTGTGGATCTACCACACACAAGGCTACTTCCCTGATTA
GCAGAACTACACACCAGGGCCAGGGGTCAGATATCCACTGACCTTTGGATGGTGCTACAAGC
TAGTACCAGTTGAGCCAGATAAGGTAGAAGAGGCCAATAAAGGAGAGAACACCAGCTTGTTA
CACCCTGTGAGCCTGCATGGGATGGATGACCCGGAGAGAGAAGTGTTAGAGTGGAGGTTTGA
CAGCCGCCTAGCATTTCATCACGTGGCCCGAGAGCTGCATCCGGAGTACTTCAAGAACTGCTG
ATATCGAGCTTGCTACAAGGGACTTTCCGCTGGGGACTTTCCAGGGAGGCGTGGCCTGGGCG
GGACTGGGGAGTGGCGAGCCCTCAGATCCTGCATATAAGCAGCTGCTTTTTGCCTGTACTGGG
TCTCTCTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGACTCT
GGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGC
TGAAGCGCGCACGGCAAGAGGCGAGGGGCGGCGACTGGTGAGTACGCCAAAAATTTTGACTA
GCGGAGGCTAGAAGGAGAGAGATGGGTGCGAGAGCGTCAGTATTAAGCGGGGGAGAATTAG
ATCGCGATGGGAAAAAATTCGGTTAAGGCCAGGGGGAAAGAAAAAATATAAATTAAAACAT
ATAGTATGGGCAAGCAGGGAGCTAGAACGATTCGCAGTTAATCCTGGCCTGTTAGAAACATC
AGAAGGCTGTAGACAAATACTGGGACAGCTACAACCATCCCTTCAGACAGGATCAGAAGAAC
TTAGATCATTATATAATACAGTAGCAACCCTCTATTGTGTGCATCAAAGGATAGAGATAAAA
GACACCAAGGAAGCTTTAGACAAGATAGAGGAAGAGCAAAACAAAAGTAAGACCACCGCACA
GCAAGCGGCCGCTGATCTTCAGACCTGGAGGAGGAGATATGAGGGACAATTGGAGAAGTGAA
TTATATAAATATAAAGTAGTAAAAATTGAACCATTAGGAGTAGCACCCACCAAGGCAAAGAG
AAGAGTGGTGCAGAGAGAAAAAAGAGCAGTGGGAATAGGAGCTTTGTTCCTTGGGTTCTTGG
GAGCAGCAGGAAGCACTATGGGCGCAGCGTCAATGACGCTGACGGTACAGGCCAGACAATTA
TTGTCTGGTATAGTGCAGCAGCAGAACAATTTGCTGAGGGCTATTGAGGCGCAACAGCATCT
GTTGCAACTCACAGTCTGGGGCATCAAGCAGCTCCAGGCAAGAATCCTGGCTGTGGAAAGAT
ACCTAAAGGATCAACAGCTCCTGGGGATTTGGGGTTGCTCTGGAAAACTCATTTGCACCACT
GCTGTGCCTTGGAATGCTAGTTGGAGTAATAAATCTCTGGAACAGATTTGGAATCACACGAC
CTGGATGGAGTGGGACAGAGAAATTAACAATTACACAAGCTTAATACACTCCTTAATTGAAG
AATCGCAAAACCAGCAAGAAAAGAATGAACAAGAATTATTGGAATTAGATAAATGGGCAAG
TTTGTGGAATTGGTTTAACATAACAAATTGGCTGTGGTATATAAAATTATTCATAATGATAG
TAGGAGGCTTGGTAGGTTTAAGAATAGTTTTTGCTGTACTTTCTATAGTGAATAGAGTTAGG
CAGGGATATTCACCATTATCGTTTCAGACCCACCTCCCAACCCCGAGGGGACCCGACAGGCCC
GAAGGAATAGAAGAAGAAGGTGGAGAGAGAGACAGAGACAGATCCATTCGATTAGTGAACG
GATCTCGACGGGATCGATTTTAAAAGAAAAGGGGGGATTGGGGGGTACAGTGCAGGGGAAA
GAATAGTAGACATAATAGCAACAGACATACAAACTAAAGAATTACAAAAACAAATTACAAA
AATTCAAAATTTTATCGATAAGCTTTGCAAAGATGGATAAAGTTTTAAACAGAGAGGAATCT
TTGCAGCTAATGGACCTTCTAGGTCTTGAAAGGAGTGGGAATTGGCTCCGGTGCCCGTCAGT
GGGCAGAGCGCACATCGCCCACAGTCCCCGAGAAGTTGGGGGGAGGGGTCGGCAATTGAACC
GGTGCCTAGAGAAGGTGGCGCGGGGTAAACTGGGAAAGTGATGTCGTGTACTGGCTCCGCCT
TTTTCCCGAGGGTGGGGGAGAACCGTATATAAGTGCAGTAGTCGCCGTGAACGTTCTTTTTC
GCAACGGGTTTGCCGCCAGAACACAGGTAAGTGCCGTGTGTGGTTCCCGCGGGCCTGGCCTCT
TTACGGGTTATGGCCCTTGCGTGCCTTGAATTACTTCCACCTGGCTGCAGTACGTGATTCTTG
ATCCCGAGCTTCGGGTTGGAAGTGGGTGGGAGAGTTCGAGGCCTTGCGCTTAAGGAGCCCCT
TCGCCTCGTGCTTGAGTTGAGGCCTGGCCTGGGCGCTGGGGCCGCCGCGTGCGAATCTGGTGG
CACCTTCGCGCCTGTCTCGCTGCTTTCGATAAGTCTCTAGCCATTTAAAATTTTTGATGACCT
GCTGCGACGCTTTTTTTCTGGCAAGATAGTCTTGTAAATGCGGGCCAAGATCTGCACACTGG
TATTTCGGTTTTTGGGGCCGCGGGCGGCGACGGGGCCCGTGCGTCCCAGCGCACATGTTCGGC
GAGGCGGGGCCTGCGAGCGCGGCCACCGAGAATCGGACGGGGGTAGTCTCAAGCTGGCCGGCC
TGCTCTGGTGCCTGGCCTCGCGCCGCCGTGTATCGCCCCGCCCTGGGCGGCAAGGCTGGCCCG
GTCGGCACCAGTTGCGTGAGCGGAAAGATGGCCGCTTCCCGGCCCTGCTGCAGGGAGCTCAAA
ATGGAGGACGCGGCGCTCGGGAGAGCGGGCGGGTGAGTCACCCACACAAAGGAAAAGGGCCT
TTCCGTCCTCAGCCGTCGCTTCATGTGACTCCACGGAGTACCGGGCGCCGTCCAGGCACCTCG
ATTAGTTCTCGAGCTTTTGGAGTACGTCGTCTTTAGGTTGGGGGGAGGGGTTTTATGCGATG
GAGTTTCCCCACACTGAGTGGGTGGAGACTGAAGTTAGGCCAGCTTGGCACTTGATGTAATT
CTCCTTGGAATTTGCCCTTTTTGAGTTTGGATCTTGGTTCATTCTCAAGCCTCAGACAGTGGT
TCAAAGTTTTTTTCTTCCATTTCAGGTGTCGTGAGGAATTCGCTAGCGCCACCATGGAGTTCT
CAAGCCCCTCTCGGGAAGAATGCCCAAAACCTCTGTCACGGGTGTCTATCATGGCTGGATCAC
TGACTGGCCTGCTGCTGCTGCAGGCCGTGAGCTGGGCCTCCGGAGCCCGGCCTTGCATCCCAA
AGTCTTTCGGCTACAGCTCCGTGGTGTGCGTGTGCAACGCCACCTATTGTGACTCCTTCGATC
CCCCTACCTTTCCCGCCCTGGGCACATTTTCTCGGTACGAGTCTACACGCAGCGGCAGGAGAA
TGGAGCTGAGCATGGGCCCTATCCAGGCCAATCACACCGGAACAGGCCTGCTGCTGACCCTGC
AGCCAGAGCAGAAGTTCCAGAAGGTGAAGGGCTTTGGAGGAGCAATGACAGACGCAGCCGCC
CTGAACATCCTGGCCCTGTCCCCACCCGCCCAGAATCTGCTGCTGAAGTCCTACTTCTCTGAG
GAGGGCATCGGCTATAACATCATCAGGGTGCCCATGGCCAGCTGCGACTTTTCCATCAGAACC
TACACATATGCCGATACCCCTGACGATTTCCAGCTGCACAATTTTTCCCTGCCAGAGGAGGAT
ACAAAGCTGAAGATCCCACTGATCCACAGGGCCCTGCAGCTGGCCCAGAGGCCCGTGAGCCTG
CTGGCCAGCCCCTGGACCTCCCCTACATGGCTGAAGACCAACGGCGCCGTGAATGGCAAGGGC
TCTCTGAAGGGACAGCCAGGCGACATCTACCACCAGACATGGGCCCGCTATTTCGTGAAGTTT
CTGGATGCCTACGCCGAGCACAAGCTGCAGTTCTGGGCCGTGACCGCAGAGAACGAGCCTTCT
GCCGGCCTGCTGAGCGGCTATCCCTTCCAGTGCCTGGGCTTTACACCTGAGCACCAGAGGGAC
TTTATCGCCAGAGATCTGGGCCCAACCCTGGCCAACTCCACACACCACAATGTGCGGCTGCTG
ATGCTGGACGATCAGCGCCTGCTGCTGCCTCACTGGGCCAAGGTGGTGCTGACCGACCCAGAG
GCCGCCAAGTACGTGCACGGCATCGCCGTGCACTGGTATCTGGATTTCCTGGCACCAGCAAAG
GCCACCCTGGGAGAGACACACAGGCTGTTCCCTAACACCATGCTGTTTGCCAGCGAGGCCTGC
GTGGGCTCCAAGTTTTGGGAGCAGTCCGTGCGGCTGGGCTCTTGGGACAGGGGCATGCAGTA
CTCCCACTCTATCATCACCAATCTGCTGTATCACGTGGTGGGCTGGACAGACTGGAACCTGGC
CCTGAATCCAGAGGGCGGCCCCAACTGGGTGAGAAATTTCGTGGATAGCCCCATCATCGTGG
ACATCACCAAGGATACATTCTACAAGCAGCCAATGTTTTATCACCTGGGCCACTTCTCTAAGT
TTATCCCAGAGGGCAGCCAGAGGGTGGGCCTGGTGGCCAGCCAGAAGAACGACCTGGATGCA
GTGGCCCTGATGCACCCTGACGGCTCCGCCGTGGTGGTGGTGCTGAATCGCTCTAGCAAGGAC
GTGCCTCTGACCATCAAGGACCCCGCCGTGGGCTTTCTGGAGACCATTTCACCCGGCTATTCT
ATTCATACCTATCTGTGGAGGAGGCAGTAACCTGCAGGGGATCCAAGCTTCAATTGTGGTCA
CTCGACAATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATTGCTTCCCG
TATGGCTTTCATTTTCTCCTCCTTGTATAAATCCTGGTTGCTGTCTCTTTATGAGGAGTTGTG
GCCCGTTGTCAGGCAACGTGGCGTGGTGTGCACTGTGTTTGCTGACGCAACCCCCACTGGTTG
GGGCATTGCCACCACCTGTCAGCTCCTTTCCGGGACTTTCGCTTTCCCCCTCCCTATTGCCACG
GCGGAACTCATCGCCGCCTGCCTTGCCCGCTGCTGGACAGGGGCTCGGCTGTTGGGCACTGAC
AATTCCGTGGTGTTGTCGGGGAAGCTGACGTCCTTTCCATGGCTGCTCGCCTGTGTTGCCACC
TGGATTCTGCGCGGGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCGCCCTCAGACGAGTC
GGATCTCCCTTTGGGCCGCCTCCCCGCCTGCTCGAGACCTAGAAAAACATGGAGCAATCACAA
GTAGCAATACAGCAGCTACCAATGCTGATTGTGCCTGGCTAGAAGCACAAGAGGAGGAGGAG
GTGGGTTTTCCAGTCACACCTCAGGTACCTTTAAGACCAATGACTTACAAGGCAGCTGTAGA
TCTTAGCCACTTTTTAAAAGAAAAGGGGGGACTGGAAGGGCTAATTCACTCCCAACGAAGAC
AAGATCTGCTTTTTGCTTGTACTGGGTCTCTCTGGTTAGACCAGATCTGAGCCTGGGAGCTCT
CTGGCTAACTAGGGAACCCACTGCTTAAGCCTCAATAAAGCTTGCCTTGAGTGCTTCAAGTA
GTGTGTGCCCGTCTGTTGTGTGACTCTGGTAACTAGAGATCCCTCAGACCCTTTTAGTCAGTG
TGGAAAATCTCTAGCAGTAGTAGTTCATGTCATCTTATTATTCAGTATTTATAACTTGCAAA
GAAATGAATATCAGAGAGTGAGAGGACGCGTTGGATGCATAGCTTGAGTATTCTATAGTGTC
ACCTAAATAGCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGAGT
GAGCTAACTCACATTAATTGCGTTGCGCTCACTGCCCGCTTTCCAGTCGGGAAACCTGTCGTG
CCAGCTGCATTAATGAATCGGCCAACGCGCGGGGAGAGGCGGTTTGCGTATTGGGCGCTCTTC
CGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAG
CAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACA
GGACTATAAAGATACCAGGCGTTTCCCCCTGGAAGCTCCCTCGTGCGCTCTCCTGTTCCGACC
CTGCCGCTTACCGGATACCTGTCCGCCTTTCTCCCTTCGGGAAGCGTGGCGCTTTCTCATAGC
TCACGCTGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAGTGGAACG
AAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAG
TTACCAATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCCCCAGTGC
TGCAATGATACCGCGAGACCCACGCTCACCGGCTCCAGATTTATCAGCAATAAACCAGCCAGC
CGGAAGGGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGCCATTG
CTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACTGCAT
AATTCTCTTACTGTCATGCCATCCGTAAGATGCTTTTCTGTGACTGGTGAGTACTCAACCAAG
TCATTCTGAGAATAGTGTATGCGGCGACCGAGTTGCTCTTGCCCGGCGTCAATACGGGATAA
TACCGCGCCACATAGCAGAACTTTAAAAGTGCTCATCATTGGAAAACGTTCTTCGGGGCGAA
AACTCTCAAGGATCTTACCGCTGTTGAGATCCAGTTCGATGTAACCCACTCGTGCACCCAACT
GATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCA
ATATTATTGAAGCATTTATCAGGGTTATTGTCTCATGAGCGGATACATATTTGAATGTATTT
AGAAAAATAAACAAATAGGGGTTCCGCGCACATTTCCCCGAAAAGTGCCACCTGATGCGGTG
TGAAATACCGCACAGATGCGTAAGGAGAAAATACCGCATCAGG
Farber disease
>pDY hASAH1
SEQ ID NO: 5
GGGCGAATTGGGCCCGACGTCGCATGCTTGGAAGGGCTAATTCACTCCCAAAGAAGACAAGA
TATCCTTGATCTGTGGATCTACCACACACAAGGCTACTTCCCTGATTAGCAGAACTACACACC
AGGGCCAGGGGTCAGATATCCACTGACCTTTGGATGGTGCTACAAGCTAGTACCAGTTGAGC
CAGATAAGGTAGAAGAGGCCAATAAAGGAGAGAACACCAGCTTGTTACACCCTGTGAGCCTG
CATGGGATGGATGACCCGGAGAGAGAAGTGTTAGAGTGGAGGTTTGACAGCCGCCTAGCATT
TCATCACGTGGCCCGAGAGCTGCATCCGGAGTACTTCAAGAACTGCTGATATCGAGCTTGCTA
CAAGGGACTTTCCGCTGGGGACTTTCCAGGGAGGCGTGGCCTGGGCGGGACTGGGGAGTGGC
GAGCCCTCAGATCCTGCATATAAGCAGCTGCTTTTTGCCTGTACTGGGTCTCTCTGGTTAGAC
CAGATCTGAGCCTGGGAGCTCTCTGGCTAACTAGGGAACCCACTGCTTAAGCCTCAATAAAGC
TTGCCTTGAGTGCTTCAAGTAGTGTGTGCCCGTCTGTTGTGTGACTCTGGTAACTAGAGATCC
CTCAGACCCTTTTAGTCAGTGTGGAAAATCTCTAGCAGTGGCGCCCGAACAGGGACTTGAAA
GCGAAAGGGAAACCAGAGGAGCTCTCTCGACGCAGGACTCGGCTTGCTGAAGCGCGCACGGC
AAGAGGCGAGGGGCGGCGACTGGTGAGTACGCCAAAAATTTTGACTAGCGGAGGCTAGAAGG
AGAGAGATGGGTGCGAGAGCGTCAGTATTAAGCGGGGGAGAATTAGATCGCGATGGGAAAA
AATTCGGTTAAGGCCAGGGGGAAAGAAAAAATATAAATTAAAACATATAGTATGGGCAAGC
AGGGAGCTAGAACGATTCGCAGTTAATCCTGGCCTGTTAGAAACATCAGAAGGCTGTAGACA
AATACTGGGACAGCTACAACCATCCCTTCAGACAGGATCAGAAGAACTTAGATCATTATATA
ATACAGTAGCAACCCTCTATTGTGTGCATCAAAGGATAGAGATAAAAGACACCAAGGAAGCT
TTAGACAAGATAGAGGAAGAGCAAAACAAAAGTAAGACCACCGCACAGCAAGCGGCCGCTGA
TCTTCAGACCTGGAGGAGGAGATATGAGGGACAATTGGAGAAGTGAATTATATAAATATAA
AGTAGTAAAAATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAA
CAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGA
CAGAGAAATTAACAATTACACAAGCTTAATACACTCCTTAATTGAAGAATCGCAAAACCAGC
AAGAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGAA
GAAGGTGGAGAGAGAGACAGAGACAGATCCATTCGATTAGTGAACGGATCTCGACGGGATCG
ATTTTAAAAGAAAAGGGGGGATTGGGGGGTACAGTGCAGGGGAAAGAATAGTAGACATAAT
AGCAACAGACATACAAACTAAAGAATTACAAAAACAAATTACAAAAATTCAAAATTTTATCG
ATAAGCTTTGCAAAGATGGATAAAGTTTTAAACAGAGAGGAATCTTTGCAGCTAATGGACCT
TCTAGGTCTTGAAAGGAGTGGGAATTGGCTCCGGTGCCCGTCAGTGGGCAGAGCGCACATCG
CCCACAGTCCCCGAGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTTCCCGAGGGTGGGG
GAGAACCGTATATAAGTGCAGTAGTCGCCGTGAACGTTCTTTTTCGCAACGGGTTTGCCGCC
AGAACACAGGTAAGTGCCGTGTGTGGTTCCCGCGGGCCTGGCCTCTTTACGGGTTATGGCCCT
TGCGTGCCTTGAATTACTTCCACCTGGCTGCAGTACGTGATTCTTGATCCCGAGCTTCGGGTT
GGAAGTGGGTGGGAGAGTTCGAGGCCTTGCGCTTAAGGAGCCCCTTCGCCTCGTGCTTGAGT
TGAGGCCTGGCCTGGGCGCTGGGGCCGCCGCGTGCGAATCTGGTGGCACCTTCGCGCCTGTCT
CGCTGCTTTCGATAAGTCTCTAGCCATTTAAAATTTTTGATGACCTGCTGCGACGCTTTTTTT
CTGGCAAGATAGTCTTGTAAATGCGGGCCAAGATCTGCACACTGGTATTTCGGTTTTTGGGG
CCGCGGGCGGCGACGGGGCCCGTGCGTCCCAGCGCACATGTTCGGCGAGGCGGGGCCTGCGAG
CGCGGCCACCGAGAATCGGACGGGGGTAGTCTCAAGCTGGCCGGCCTGCTCTGGTGCCTGGCC
TCGCGCCGCCGTGTATCGCCCCGCCCTGGGCGGCAAGGCTGGCCCGGTCGGCACCAGTTGCGT
GAGCGGAAAGATGGCCGCTTCCCGGCCCTGCTGCAGGGAGCTCAAAATGGAGGACGCGGCGC
TCGGGAGAGCGGGCGGGTGAGTCACCCACACAAAGGAAAAGGGCCTTTCCGTCCTCAGCCGTC
GCTTCATGTGACTCCACGGAGTACCGGGCGCCGTCCAGGCACCTCGATTAGTTCTCGAGCTTT
TGGAGTACGTCGTCTTTAGGTTGGGGGGAGGGGTTTTATGCGATGGAGTTTCCCCACACTGA
GTGGGTGGAGACTGAAGTTAGGCCAGCTTGGCACTTGATGTAATTCTCCTTGGAATTTGCCC
TTTTTGAGTTTGGATCTTGGTTCATTCTCAAGCCTCAGACAGTGGTTCAAAGTTTTTTTCTTC
CATTTCAGGTGTCGTGAGGAATTCTGCAGTCGACGCCACCATGCCGGGCCGGAGTTGCGTCGC
CTTAGTCCTCCTGGCTGCCGCCGTCAGCTGTGCCGTCGCGCAGCACGCGCCGCCGTGGACAGA
GGACTGCAGAAAATCAACCTATCCTCCTTCAGGACCAACGTACAGAGGTGCAGTTCCATGGT
ACACCATAAATCTTGACTTACCACCCTACAAAAGATGGCATGAATTGATGCTTGACAAGGCA
CCAGTGCTAAAGGTTATAGTGAATTCTCTGAAGAATATGATAAATACATTCGTGCCAAGTGG
AAAAATTATGCAGGTGGTGGATGAAAAATTGCCTGGCCTACTTGGCAACTTTCCTGGCCCTT
TTGAAGAGGAAATGAAGGGTATTGCCGCTGTTACTGATATACCTTTAGGAGAGATTATTTCA
TTCAATATTTTTTATGAATTATTTACCATTTGTACTTCAATAGTAGCAGAAGACAAAAAAGG
TCATCTAATACATGGGAGAAACATGGATTTTGGAGTATTTCTTGGGTGGAACATAAATAATG
ATACCTGGGTCATAACTGAGCAACTAAAACCTTTAACAGTGAATTTGGATTTCCAAAGAAAC
AACAAAACTGTCTTCAAGGCTTCAAGCTTTGCTGGCTATGTGGGCATGTTAACAGGATTCAA
ACCAGGACTGTTCAGTCTTACACTGAATGAACGTTTCAGTATAAATGGTGGTTATCTGGGTA
TTCTAGAATGGATTCTGGGAAAGAAAGATGTCATGTGGATAGGGTTCCTCACTAGAACAGTT
CTGGAAAATAGCACAAGTTATGAAGAAGCCAAGAATTTATTGACCAAGACCAAGATATTGGC
CCCAGCCTACTTTATCCTGGGAGGCAACCAGTCTGGGGAAGGTTGTGTGATTACACGAGACA
GAAAGGAATCATTGGATGTATATGAACTCGATGCTAAGCAGGGTAGATGGTATGTGGTACAA
ACAAATTATGACCGTTGGAAACATCCCTTCTTCCTTGATGATCGCAGAACGCCTGCAAAGAT
GTGTCTGAACCGCACCAGCCAAGAGAATATCTCATTTGAAACCATGTATGATGTCCTGTCAA
CAAAACCTGTCCTCAACAAGCTGACCGTATACACAACCTTGATAGATGTTACCAAAGGTCAA
TTCGAAACTTACCTGCGGGACTGCCCTGACCCTTGTATAGGTTGGTGAGCGGCCGCCTCGAGG
ATCCAAGCTTCAATTGTGGTCACTCGACAATCAACCTCTGGATTACAAAATTTGTGAAAGAT
TGACTGGTATTCTTAACTATGTTGCTCCTTTTACGCTATGTGGATACGCTGCTTTAATGCCTT
TGTATCATGCTATTGCTTCCCGTATGGCTTTCATTTTCTCCTCCTTGTATAAATCCTGGTTGC
TGTCTCTTTATGAGGAGTTGTGGCCCGTTGTCAGGCAACGTGGCGTGGTGTGCACTGTGTTT
GCTGACGCAACCCCCACTGGTTGGGGCATTGCCACCACCTGTCAGCTCCTTTCCGGGACTTTC
GCTTTCCCCCTCCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAGCTGACGTCCTTTCCAT
GGCTGCTCGCCTGTGTTGCCACCTGGATTCTGCGCGGGACGTCCTTCTGCTACGTCCCTTCGG
CCCTCAATCCAGCGGACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCGCCTGCTCGAGACCTA
GAAAAACATGGAGCAATCACAAGTAGCAATACAGCAGCTACCAATGCTGATTGTGCCTGGCT
AGAAGCACAAGAGGAGGAGGAGGTGGGTTTTCCAGTCACACCTCAGGTACCTTTAAGACCAA
TGACTTACAAGGCAGCTGTAGATCTTAGCCACTTTTTAAAAGAAAAGGGGGGACTGGAAGGG
CTAATTCACTCCCAACGAAGACAAGATCTGCTTTTTGCTTGTACTGGGTCTCTCTGGTTAGAC
CAGATCTGAGCCTGGGAGCTCTCTGGCTAACTAGGGAACCCACTGCTTAAGCCTCAATAAAGC
TTGCCTTGAGTGCTTCAAGTAGTGTGTGCCCGTCTGTTGTGTGACTCTGGTAACTAGAGATCC
CTCAGACCCTTTTAGTCAGTGTGGAAAATCTCTAGCAGTAGTAGTTCATGTCATCTTATTAT
TCAGTATTTATAACTTGCAAAGAAATGAATATCAGAGAGTGAGAGGACGCGTTGGATGCATA
GCTTGAGTATTCTATAGTGTCACCTAAATAGCTTGGCGTAATCATGGTCATAGCTGTTTCCT
GTGTGAAATTGTTATCCGCTCACAATTCCACACAACATACGAGCCGGAAGCATAAAGTGTAA
AGCCTGGGGTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGGGGAGAGGCG
GTTTGCGTATTGGGCGCTCTTCCGCTTCCTCGCTCACTGACTCGCTGCGCTCGGTCGTTCGGC
TGCGGCGAGCGGTATCAGCTCACTCAAAGGCGGTAATACGGTTATCCACAGAATCAGGGGAT
AACGCAGGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTCAAG
TCAGAGGTGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCCCTTCGGGA
AGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAACTACGG
CTACACTAGAAGAACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATCAAAAA
GGATCTTCACCTAGATCCTTTTAAATTAAAAATGAAGTTTTAAATCAATCTAAAGTATATAT
GAGTAAACTTGGTCTGACAGTTACCAATGCTTAATCAGTGAGGCACCTATCTCAGCGATCTG
TCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAATAGT
TTGCGCAACGTTGTTGCCATTGCTACAGGCATCGTGGTGTCACGCTCGTCGTTTGGTATGGCT
TCATTCAGCTCCGGTTCCCAACGATCAAGGCGAGTTACATGATCCCCCATGTTGTGCAAAAAA
GCGGTTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCATCATTG
GAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGA
GCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCG
GATACATATTTGAATGTATTTAGAAAAATAAACAAATAGGGGTTCCGCGCACATTTCCCCGA
AAAGTGCCACCTGATGCGGTGTGAAATACCGCACAGATGCGTAAGGAGAAAATACCGCATCA
GGAAATTGTAAGCGTTAATATTTTGTTAAAATTCGCGTTAAATTTTTGTTAAATCAGCTCAT
TTTTTAACCAATAGGCCGAAATCGGCAAAATCCCTTATAAATCAAAAGAATAGACCGAGATA
GGGTTGAGTGTTGTTCCAGTTTGGAACAAGAGTCCACTATTAAAGAACGTGGACTCCAACGT
CAAAGGGCGAAAAACCGTCTATCAGGGCGATGGCCCACTACGTGAACCATCACCCTAATCAA
GTTTTTTGGGGTCGAGGTGCCGTAAAGCACTAAATCGGAACCCTAAAGGGAGCCCCCGATTT
AGAGCTTGACGGGGAAAGCCGGCGAACGTGGCGAGAAAGGAAGGGAAGAAAGCGAAAGGAG
CGGGCGCTAGGGCGCTGGCAAGTGTAGCGGTCACGCTGCGCGTAACCACCACACCCGCCGCGC
TTAATGCGCCGCTACAGGGCGCGTCCATTCGCCATTCAGGCTGCGCAACTGTTGGGAAGGGCG
ATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTG
TAATACGACTCACTATA
Pompe disease (vector encoding GAA)
>pDY co hGAA
SEQ ID NO: 6
AAATTGTAAGCGTTAATATTTTGTTAAAATTCGCGTTAAATTTTTGTTAAATCAGCTCATTT
TTTAACCAATAGGCCGAAATCGGCAAAATCCCTTATAAATCAAAAGAATAGACCGAGATAGG
GTTGAGTGTTGTTCCAGTTTGGAACAAGAGTCCACTATTAAAGAACGTGGACTCCAACGTCA
AAGGGCGAAAAACCGTCTATCAGGGCGATGGCCCACTACGTGAACCATCACCCTAATCAAGT
TTTTTGGGGTCGAGGTGCCGTAAAGCACTAAATCGGAACCCTAAAGGGAGCCCCCGATTTAG
AGCTTGACGGGGAAAGCCGGCGAACGTGGCGAGAAAGGAAGGGAAGAAAGCGAAAGGAGCG
GGCGCTAGGGCGCTGGCAAGTGTAGCGGTCACGCTGCGCGTAACCACCACACCCGCCGCGCTT
AATGCGCCGCTACAGGGCGCGTCCATTCGCCATTCAGGCTGCGCAACTGTTGGGAAGGGCGAT
CGGTGCGGGCCTCTTCGCTATTACGCCAGCTGGCGAAAGGGGGATGTGCTGCAAGGCGATTA
AGTTGGGTAACGCCAGGGTTTTCCCAGTCACGACGTTGTAAAACGACGGCCAGTGAATTGTA
ATACGACTCACTATAGGGCGAATTGGGCCCGACGTCGCATGCTTGGAAGGGCTAATTCACTCC
CAAAGAAGACAAGATATCCTTGATCTGTGGATCTACCACACACAAGGCTACTTCCCTGATTA
GCAGAACTACACACCAGGGCCAGGGGTCAGATATCCACTGACCTTTGGATGGTGCTACAAGC
TAGTACCAGTTGAGCCAGATAAGGTAGAAGAGGCCAATAAAGGAGAGAACACCAGCTTGTTA
CACCCTGTGAGCCTGCATGGGATGGATGACCCGGAGAGAGAAGTGTTAGAGTGGAGGTTTGA
CAGCCGCCTAGCATTTCATCACGTGGCCCGAGAGCTGCATCCGGAGTACTTCAAGAACTGCTG
ATATCGAGCTTGCTACAAGGGACTTTCCGCTGGGGACTTTCCAGGGAGGCGTGGCCTGGGCG
GGACTGGGGAGTGGCGAGCCCTCAGATCCTGCATATAAGCAGCTGCTTTTTGCCTGTACTGGG
TCTCTCTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGACTCT
GGTAACTAGAGATCCCTCAGACCCTTTTAGTCAGTGTGGAAAATCTCTAGCAGTGGCGCCCG
AACAGGGACTTGAAAGCGAAAGGGAAACCAGAGGAGCTCTCTCGACGCAGGACTCGGCTTGC
TGAAGCGCGCACGGCAAGAGGCGAGGGGCGGCGACTGGTGAGTACGCCAAAAATTTTGACTA
GCGGAGGCTAGAAGGAGAGAGATGGGTGCGAGAGCGTCAGTATTAAGCGGGGGAGAATTAG
ATCGCGATGGGAAAAAATTCGGTTAAGGCCAGGGGGAAAGAAAAAATATAAATTAAAACAT
ATAGTATGGGCAAGCAGGGAGCTAGAACGATTCGCAGTTAATCCTGGCCTGTTAGAAACATC
AGAAGGCTGTAGACAAATACTGGGACAGCTACAACCATCCCTTCAGACAGGATCAGAAGAAC
TTAGATCATTATATAATACAGTAGCAACCCTCTATTGTGTGCATCAAAGGATAGAGATAAAA
GACACCAAGGAAGCTTTAGACAAGATAGAGGAAGAGCAAAACAAAAGTAAGACCACCGCACA
GCAAGCGGCCGCTGATCTTCAGACCTGGAGGAGGAGATATGAGGGACAATTGGAGAAGTGAA
TTATATAAATATAAAGTAGTAAAAATTGAACCATTAGGAGTAGCACCCACCAAGGCAAAGAG
AAGAGTGGTGCAGAGAGAAAAAAGAGCAGTGGGAATAGGAGCTTTGTTCCTTGGGTTCTTGG
GAGCAGCAGGAAGCACTATGGGCGCAGCGTCAATGACGCTGACGGTACAGGCCAGACAATTA
TTGTCTGGTATAGTGCAGCAGCAGAACAATTTGCTGAGGGCTATTGAGGCGCAACAGCATCT
GTTGCAACTCACAGTCTGGGGCATCAAGCAGCTCCAGGCAAGAATCCTGGCTGTGGAAAGAT
ACCTAAAGGATCAACAGCTCCTGGGGATTTGGGGTTGCTCTGGAAAACTCATTTGCACCACT
GCTGTGCCTTGGAATGCTAGTTGGAGTAATAAATCTCTGGAACAGATTTGGAATCACACGAC
CTGGATGGAGTGGGACAGAGAAATTAACAATTACACAAGCTTAATACACTCCTTAATTGAAG
AATCGCAAAACCAGCAAGAAAAGAATGAACAAGAATTATTGGAATTAGATAAATGGGCAAG
TTTGTGGAATTGGTTTAACATAACAAATTGGCTGTGGTATATAAAATTATTCATAATGATAG
TAGGAGGCTTGGTAGGTTTAAGAATAGTTTTTGCTGTACTTTCTATAGTGAATAGAGTTAGG
CAGGGATATTCACCATTATCGTTTCAGACCCACCTCCCAACCCCGAGGGGACCCGACAGGCCC
GAAGGAATAGAAGAAGAAGGTGGAGAGAGAGACAGAGACAGATCCATTCGATTAGTGAACG
GATCTCGACGGGATCGATTTTAAAAGAAAAGGGGGGATTGGGGGGTACAGTGCAGGGGAAA
GAATAGTAGACATAATAGCAACAGACATACAAACTAAAGAATTACAAAAACAAATTACAAA
AATTCAAAATTTTATCGATAAGCTTTGCAAAGATGGATAAAGTTTTAAACAGAGAGGAATCT
TTGCAGCTAATGGACCTTCTAGGTCTTGAAAGGAGTGGGAATTGGCTCCGGTGCCCGTCAGT
GGGCAGAGCGCACATCGCCCACAGTCCCCGAGAAGTTGGGGGGAGGGGTCGGCAATTGAACC
GGTGCCTAGAGAAGGTGGCGCGGGGTAAACTGGGAAAGTGATGTCGTGTACTGGCTCCGCCT
TTTTCCCGAGGGTGGGGGAGAACCGTATATAAGTGCAGTAGTCGCCGTGAACGTTCTTTTTC
GCAACGGGTTTGCCGCCAGAACACAGGTAAGTGCCGTGTGTGGTTCCCGCGGGCCTGGCCTCT
TTACGGGTTATGGCCCTTGCGTGCCTTGAATTACTTCCACCTGGCTGCAGTACGTGATTCTTG
ATCCCGAGCTTCGGGTTGGAAGTGGGTGGGAGAGTTCGAGGCCTTGCGCTTAAGGAGCCCCT
TCGCCTCGTGCTTGAGTTGAGGCCTGGCCTGGGCGCTGGGGCCGCCGCGTGCGAATCTGGTGG
CACCTTCGCGCCTGTCTCGCTGCTTTCGATAAGTCTCTAGCCATTTAAAATTTTTGATGACCT
GCTGCGACGCTTTTTTTCTGGCAAGATAGTCTTGTAAATGCGGGCCAAGATCTGCACACTGG
TATTTCGGTTTTTGGGGCCGCGGGCGGCGACGGGGCCCGTGCGTCCCAGCGCACATGTTCGGC
GAGGCGGGGCCTGCGAGCGCGGCCACCGAGAATCGGACGGGGGTAGTCTCAAGCTGGCCGGCC
TGCTCTGGTGCCTGGCCTCGCGCCGCCGTGTATCGCCCCGCCCTGGGCGGCAAGGCTGGCCCG
GTCGGCACCAGTTGCGTGAGCGGAAAGATGGCCGCTTCCCGGCCCTGCTGCAGGGAGCTCAAA
ATGGAGGACGCGGCGCTCGGGAGAGCGGGCGGGTGAGTCACCCACACAAAGGAAAAGGGCCT
TTCCGTCCTCAGCCGTCGCTTCATGTGACTCCACGGAGTACCGGGCGCCGTCCAGGCACCTCG
ATTAGTTCTCGAGCTTTTGGAGTACGTCGTCTTTAGGTTGGGGGGAGGGGTTTTATGCGATG
GAGTTTCCCCACACTGAGTGGGTGGAGACTGAAGTTAGGCCAGCTTGGCACTTGATGTAATT
CTCCTTGGAATTTGCCCTTTTTGAGTTTGGATCTTGGTTCATTCTCAAGCCTCAGACAGTGGT
TCAAAGTTTTTTTCTTCCATTTCAGGTGTCGTGAGGAATTCGCCACCATGGGCGTGAGGCACC
CCCCTTGCTCTCACAGGCTGCTGGCCGTGTGCGCACTGGTGAGCCTGGCCACCGCCGCCCTGC
TGGGCCACATCCTGCTGCACGACTTCCTGCTGGTGCCCAGGGAGCTGTCCGGCAGCTCCCCAG
TGCTGGAGGAGACCCACCCAGCACACCAGCAGGGCGCCTCTCGGCCAGGCCCCCGCGATGCAC
AGGCACACCCAGGCCGGCCCCGCGCCGTGCCAACCCAGTGCGACGTGCCACCCAACAGCCGGT
TTGACTGTGCCCCCGATAAGGCCATCACACAGGAGCAGTGCGAGGCCAGGGGCTGCTGTTAT
ATCCCTGCAAAGCAGGGCCTCCAGGGCGCCCAGATGGGACAGCCATGGTGTTTCTTTCCTCCA
TCTTACCCCAGCTATAAGCTGGAGAATCTGTCTAGCTCCGAGATGGGCTACACAGCCACCCTG
ACAAGAACCACACCAACATTCTTTCCCAAGGACATCCTGACCCTGCGGCTGGACGTGATGATG
GAGACAGAGAACCGCCTGCACTTCACCATCAAGGACCCCGCCAATAGGAGATATGAGGTGCC
TCTGGAGACCCCACACGTGCACTCTCGGGCCCCTAGCCCACTGTACTCCGTGGAGTTCTCTGA
GGAGCCATTTGGCGTGATCGTGCGGCGCCAGCTGGATGGACGCGTGCTGCTGAACACCACAGT
GGCCCCCCTGTTCTTTGCCGACCAGTTCCTCCAGCTGAGCACATCCCTGCCCTCCCAGTATATC
ACCGGCCTGGCCGAGCACCTGTCTCCTCTGATGCTGTCTACCAGCTGGACAAGGATCACCCTG
TGGAACAGAGACCTGGCACCAACCCCTGGCGCAAATCTGTACGGCAGCCACCCTTTCTATCTG
GCCCTGGAGGATGGAGGCTCCGCCCACGGCGTGTTTCTGCTGAACTCTAATGCCATGGACGTG
GTGCTCCAGCCAAGCCCCGCCCTGTCCTGGCGGTCTACCGGCGGCATCCTGGACGTGTACATC
TTCCTGGGCCCTGAGCCAAAGTCCGTGGTGCAGCAGTACCTGGACGTGGTGGGCTATCCTTTC
ATGCCCCCTTACTGGGGACTGGGATTTCACCTGTGCCGCTGGGGCTATTCTAGCACAGCCATC
ACCCGGCAGGTGGTGGAGAACATGACCCGCGCCCACTTTCCACTGGATGTGCAGTGGAATGAC
CTGGATTACATGGACTCCAGGAGAGACTTCACCTTCAACAAGGACGGCTTCAGGGATTTTCCC
GCCATGGTGCAGGAGCTGCACCAGGGCGGCCGGCGCTACATGATGATCGTGGACCCCGCCATC
TCCTCTAGCGGACCTGCCGGCAGCTACAGACCATATGACGAGGGCCTGAGGAGAGGCGTGTTC
ATCACAAACGAGACCGGCCAGCCTCTGATCGGCAAGGTCTGGCCAGGCTCCACCGCCTTCCCA
GACTTCACCAATCCAACCGCCCTGGCCTGGTGGGAGGACATGGTGGCCGAGTTCCACGACCAG
GTGCCTTTTGATGGCATGTGGATCGACATGAACGAGCCATCTAATTTCATCAGGGGCAGCGA
GGACGGCTGCCCCAACAATGAGCTGGAGAACCCACCATATGTGCCTGGCGTGGTGGGAGGCA
CCCTCCAGGCAGCAACCATCTGTGCCTCCTCTCACCAGTTTCTGTCTACACACTATAACCTGC
ACAATCTGTACGGACTGACCGAGGCAATCGCCAGCCACAGAGCCCTGGTGAAGGCCAGGGGC
ACAAGACCTTTCGTGATCTCCAGGTCTACCTTTGCCGGACACGGCAGATACGCAGGACACTGG
ACCGGCGACGTGTGGAGCAGCTGGGAGCAGCTGGCCTCTAGCGTGCCAGAGATCCTCCAGTTC
AACCTGCTGGGCGTGCCCCTGGTGGGAGCAGACGTGTGCGGCTTTCTGGGCAATACATCCGAG
GAGCTGTGCGTGAGGTGGACCCAGCTGGGAGCCTTCTATCCCTTCATGCGCAACCACAATAGC
CTGCTGTCCCTGCCTCAGGAGCCATACAGCTTCTCCGAGCCTGCACAGCAGGCAATGAGGAAG
GCCCTGACACTGCGCTATGCCCTGCTGCCACACCTGTACACCCTGTTTCACCAGGCACACGTG
GCAGGAGAGACAGTGGCCCGGCCCCTGTTCCTGGAGTTTCCTAAGGATTCCTCTACCTGGACA
GTGGACCACCAGCTGCTGTGGGGAGAGGCCCTGCTGATCACCCCCGTGCTCCAGGCAGGCAAG
GCAGAGGTGACAGGCTATTTCCCTCTGGGCACATGGTACGACCTCCAGACCGTGCCAGTGGAG
GCCCTGGGCAGCCTGCCTCCACCACCTGCCGCCCCCCGCGAGCCTGCCATCCACTCCGAGGGAC
AGTGGGTGACACTGCCAGCACCTCTGGACACCATCAACGTGCACCTGAGGGCCGGCTATATCA
TCCCCCTCCAGGGCCCTGGCCTGACCACAACCGAGTCCAGACAGCAGCCAATGGCCCTGGCCG
TGGCCCTGACCAAGGGAGGCGAGGCCAGGGGCGAGCTGTTCTGGGACGATGGCGAGTCTCTG
GAGGTGCTGGAGAGAGGCGCCTACACACAGGTCATCTTCCTGGCCAGGAACAATACAATCGT
GAATGAGCTGGTGAGAGTGACCTCTGAGGGAGCAGGACTCCAGCTCCAGAAGGTGACAGTGC
TGGGAGTGGCAACCGCACCACAGCAGGTGCTGAGCAACGGCGTGCCCGTGAGCAATTTCACA
TACTCCCCTGATACCAAGGTGCTGGACATCTGCGTGAGCCTGCTGATGGGCGAGCAGTTTCTG
GTGTCCTGGTGTTGAGAACCCGGGATCCAAGCTTCAATTGTGGTCACTCGACAATCAACCTCT
GGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGGCATTGCCACCACCT
GTCAGCTCCTTTCCGGGACTTTCGCTTTCCCCCTCCCTATTGCCACGGCGGAACTCATCGCCGC
CTGCCTTGCCCGCTGCTGGACAGGGGCTCGGCTGTTGGGCACTGACAATTCCGTGGTGTTGTC
GGGGAAGCTGACGTCCTTTCCATGGCTGCTCGCCTGTGTTGCCACCTGGATTCTGCGCGGGAC
GTCCTTCTGCTACGTCCCTTCGGCCCTCAATCCAGCGGACCTTCCTTCCCGCGGCCTGCTGCCG
GCTCTGCGGCCTCTTCCGCGTCTTCGCCTTCGCCCTCAGACGAGTCGGATCTCCCTTTGGGCCG
CCTCCCCGCCTGCTCGAGACCTAGAAAAACATGGAGCAATCACAAGTAGCAATACAGCAGCT
ACCAATGCTGATTGTGCCTGGCTAGAAGCACAAGAGGAGGAGGAGGTGGGTTTTCCAGTCAC
ACCTCAGGTACCTTTAAGACCAATGACTTACAAGGCAGCTGTAGATCTTAGCCACTTTTTAA
AAGAAAAGGGGGGACTGGAAGGGCTAATTCACTCCCAACGAAGACAAGATCTGCTTTTTGCT
TGTACTGGGTCTCTCTGGTTAGACCAGATCTGAGCCTGGGAGCTCTCTGGCTAACTAGGGAAC
CCACTGCTTAAGCCTCAATAAAGCTTGCCTTGAGTGCTTCAAGTAGTGTGTGCCCGTCTGTTG
TGTGACTCTGGTAACTAGAGATCCCTCAGACCCTTTTAGTCAGTGTGGAAAATCTCTAGCAG
TAGTAGTTCATGTCATCTTATTATTCAGTATTTATAACTTGCAAAGAAATGAATATCAGAGA
GTGAGAGGACGCGTTGGATGCATAGCTTGAGTATTCTATAGTGTCACCTAAATAGCTTGGCG
TAATCATGGTCATAGCTGTTTCCTGTGTGAAATTGTTATCCGCTCACAATTCCACACAACATA
CGAGCCGGAAGCATAAAGTGTAAAGCCTGGGGTGCCTAATGAGTGAGCTAACTCACATTAAT
TGCGTTGCGCTCACTGCCCGCTTTCCAGTCGGGAAACCTGTCGTGCCAGCTGCATTAATGAAT
CGGCCAACGCGCGGGGAGAGGCGGTTTGCGTATTGGGCGCTCTTCCGCTTCCTCGCTCACTGA
CTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGG
CCAGGAACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCGACC
GCTGCGCCTTATCCGGTAACTATCGTCTTGAGTCCAACCCGGTAAGACACGACTTATCGCCAC
TGGCAGCAGCCACTGGTAACAGGATTAGCAGAGCGAGGTATGTAGGCGGTGCTACAGAGTTC
TTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGAT
TTTGGTCATGAGATTATCAAAAAGGATCTTCACCTAGATCCTTTTAAATTAAAAATGAAGTT
TTAAATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGCGAGA
CCCACGCTCACCGGCTCCAGATTTATCAGCAATAAACCAGCCAGCCGGAAGGGCCGAGCGCAG
AAGTGGTCCTGCAACTTTATCCGCCTCCATCCAGTCTATTAATTGTTGCCGGGAAGCTAGAGT
AAGTAGTTCGCCAGTTAATAGTTTGCGCAACGTTGTTGCCATTGCTACAGGCATCGTGGTGT
CACGCTCGTCGTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTTGTCAGAAGTA
AGTTGGCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAATAGTGT
ATGCGGCGACCGAGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCAAGGATCTTACC
GCTGTTGAGATCCAGTTCGATGTAACCCACTCGTGCACCCAACTGATCTTCAGCATCTTTTAC
TTTCACCAGCGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAGCATTTAT
CAGGGTTATTGTCTCATGAGCGGATACATATTTGAATGTATTTAGAAAAATAAACAAATAGG
GGTTCCGCGCACATTTCCCCGAAAAGTGCCACCTGATGCGGTGTGAAATACCGCACAGATGCG
TAAGGAGAAAATACCGCATCAGG
GBA transgene
>co.hGBA
SEQ ID NO: 7
ATGGAGTTCTCAAGCCCCTCTCGGGAAGAATGCCCAAAACCTCTGTCACGGGTGTCTATCATG
GCTGGATCACTGACTGGCCTGCTGCTGCTGCAGGCCGTGAGCTGGGCCTCCGGAGCCCGGCCT
TGCATCCCAAAGTCTTTCGGCTACAGCTCCGTGGTGTGCGTGTGCAACGCCACCTATTGTGAC
TCCTTCGATCCCCCTACCTTTCCCGCCCTGGGCACATTTTCTCGGTACGAGTCTACACGCAGC
GGCAGGAGAATGGAGCTGAGCATGGGCCCTATCCAGGCCAATCACACCGGAACAGGCCTGCT
GCTGACCCTGCAGCCAGAGCAGAAGTTCCAGAAGGTGAAGGGCTTTGGAGGAGCAATGACAG
ACGCAGCCGCCCTGAACATCCTGGCCCTGTCCCCACCCGCCCAGAATCTGCTGCTGAAGTCCT
ACTTCTCTGAGGAGGGCATCGGCTATAACATCATCAGGGTGCCCATGGCCAGCTGCGACTTTT
CCATCAGAACCTACACATATGCCGATACCCCTGACGATTTCCAGCTGCACAATTTTTCCCTGC
CAGAGGAGGATACAAAGCTGAAGATCCCACTGATCCACAGGGCCCTGCAGCTGGCCCAGAGG
CCCGTGAGCCTGCTGGCCAGCCCCTGGACCTCCCCTACATGGCTGAAGACCAACGGCGCCGTG
AATGGCAAGGGCTCTCTGAAGGGACAGCCAGGCGACATCTACCACCAGACATGGGCCCGCTAT
TTCGTGAAGTTTCTGGATGCCTACGCCGAGCACAAGCTGCAGTTCTGGGCCGTGACCGCAGAG
AACGAGCCTTCTGCCGGCCTGCTGAGCGGCTATCCCTTCCAGTGCCTGGGCTTTACACCTGAG
CACCAGAGGGACTTTATCGCCAGAGATCTGGGCCCAACCCTGGCCAACTCCACACACCACAAT
GTGCGGCTGCTGATGCTGGACGATCAGCGCCTGCTGCTGCCTCACTGGGCCAAGGTGGTGCTG
ACCGACCCAGAGGCCGCCAAGTACGTGCACGGCATCGCCGTGCACTGGTATCTGGATTTCCTG
GCACCAGCAAAGGCCACCCTGGGAGAGACACACAGGCTGTTCCCTAACACCATGCTGTTTGCC
AGCGAGGCCTGCGTGGGCTCCAAGTTTTGGGAGCAGTCCGTGCGGCTGGGCTCTTGGGACAG
GGGCATGCAGTACTCCCACTCTATCATCACCAATCTGCTGTATCACGTGGTGGGCTGGACAGA
CTGGAACCTGGCCCTGAATCCAGAGGGCGGCCCCAACTGGGTGAGAAATTTCGTGGATAGCCC
CATCATCGTGGACATCACCAAGGATACATTCTACAAGCAGCCAATGTTTTATCACCTGGGCCA
CTTCTCTAAGTTTATCCCAGAGGGCAGCCAGAGGGTGGGCCTGGTGGCCAGCCAGAAGAACG
ACCTGGATGCAGTGGCCCTGATGCACCCTGACGGCTCCGCCGTGGTGGTGGTGCTGAATCGCT
CTAGCAAGGACGTGCCTCTGACCATCAAGGACCCCGCCGTGGGCTTTCTGGAGACCATTTCAC
CCGGCTATTCTATTCATACCTATCTGTGGAGGAGGCAGTAA
ASAH1 transgene
>hASAH1
SEQ ID NO: 8
ATGCCGGGCCGGAGTTGCGTCGCCTTAGTCCTCCTGGCTGCCGCCGTCAGCTGTGCCGTCGCG
CAGCACGCGCCGCCGTGGACAGAGGACTGCAGAAAATCAACCTATCCTCCTTCAGGACCAACG
TACAGAGGTGCAGTTCCATGGTACACCATAAATCTTGACTTACCACCCTACAAAAGATGGCA
TGAATTGATGCTTGACAAGGCACCAGTGCTAAAGGTTATAGTGAATTCTCTGAAGAATATGA
TAAATACATTCGTGCCAAGTGGAAAAATTATGCAGGTGGTGGATGAAAAATTGCCTGGCCTA
CTTGGCAACTTTCCTGGCCCTTTTGAAGAGGAAATGAAGGGTATTGCCGCTGTTACTGATAT
ACCTTTAGGAGAGATTATTTCATTCAATATTTTTTATGAATTATTTACCATTTGTACTTCAA
TAGTAGCAGAAGACAAAAAAGGTCATCTAATACATGGGAGAAACATGGATTTTGGAGTATTT
CTTGGGTGGAACATAAATAATGATACCTGGGTCATAACTGAGCAACTAAAACCTTTAACAGT
GAATTTGGATTTCCAAAGAAACAACAAAACTGTCTTCAAGGCTTCAAGCTTTGCTGGCTATG
TGGGCATGTTAACAGGATTCAAACCAGGACTGTTCAGTCTTACACTGAATGAACGTTTCAGT
ATAAATGGTGGTTATCTGGGTATTCTAGAATGGATTCTGGGAAAGAAAGATGTCATGTGGAT
AGGGTTCCTCACTAGAACAGTTCTGGAAAATAGCACAAGTTATGAAGAAGCCAAGAATTTAT
TGACCAAGACCAAGATATTGGCCCCAGCCTACTTTATCCTGGGAGGCAACCAGTCTGGGGAA
GGTTGTGTGATTACACGAGACAGAAAGGAATCATTGGATGTATATGAACTCGATGCTAAGCA
GGGTAGATGGTATGTGGTACAAACAAATTATGACCGTTGGAAACATCCCTTCTTCCTTGATG
ATCGCAGAACGCCTGCAAAGATGTGTCTGAACCGCACCAGCCAAGAGAATATCTCATTTGAA
ACCATGTATGATGTCCTGTCAACAAAACCTGTCCTCAACAAGCTGACCGTATACACAACCTTG
ATAGATGTTACCAAAGGTCAATTCGAAACTTACCTGCGGGACTGCCCTGACCCTTGTATAGG
TTGGTGA
(GAA transgene)
>co.hGAA
SEQ ID NO: 9
ATGGGCGTGAGGCACCCCCCTTGCTCTCACAGGCTGCTGGCCGTGTGCGCACTGGTGAGCCTG
GCCACCGCCGCCCTGCTGGGCCACATCCTGCTGCACGACTTCCTGCTGGTGCCCAGGGAGCTG
TCCGGCAGCTCCCCAGTGCTGGAGGAGACCCACCCAGCACACCAGCAGGGCGCCTCTCGGCCA
GGCCCCCGCGATGCACAGGCACACCCAGGCCGGCCCCGCGCCGTGCCAACCCAGTGCGACGTG
CCACCCAACAGCCGGTTTGACTGTGCCCCCGATAAGGCCATCACACAGGAGCAGTGCGAGGCC
AGGGGCTGCTGTTATATCCCTGCAAAGCAGGGCCTCCAGGGCGCCCAGATGGGACAGCCATGG
TGTTTCTTTCCTCCATCTTACCCCAGCTATAAGCTGGAGAATCTGTCTAGCTCCGAGATGGGC
TACACAGCCACCCTGACAAGAACCACACCAACATTCTTTCCCAAGGACATCCTGACCCTGCGG
CTGGACGTGATGATGGAGACAGAGAACCGCCTGCACTTCACCATCAAGGACCCCGCCAATAG
GAGATATGAGGTGCCTCTGGAGACCCCACACGTGCACTCTCGGGCCCCTAGCCCACTGTACTC
CGTGGAGTTCTCTGAGGAGCCATTTGGCGTGATCGTGCGGCGCCAGCTGGATGGACGCGTGCT
GCTGAACACCACAGTGGCCCCCCTGTTCTTTGCCGACCAGTTCCTCCAGCTGAGCACATCCCT
GCCCTCCCAGTATATCACCGGCCTGGCCGAGCACCTGTCTCCTCTGATGCTGTCTACCAGCTG
GACAAGGATCACCCTGTGGAACAGAGACCTGGCACCAACCCCTGGCGCAAATCTGTACGGCAG
CCACCCTTTCTATCTGGCCCTGGAGGATGGAGGCTCCGCCCACGGCGTGTTTCTGCTGAACTC
TAATGCCATGGACGTGGTGCTCCAGCCAAGCCCCGCCCTGTCCTGGCGGTCTACCGGCGGCAT
CCTGGACGTGTACATCTTCCTGGGCCCTGAGCCAAAGTCCGTGGTGCAGCAGTACCTGGACGT
GGTGGGCTATCCTTTCATGCCCCCTTACTGGGGACTGGGATTTCACCTGTGCCGCTGGGGCTA
TTCTAGCACAGCCATCACCCGGCAGGTGGTGGAGAACATGACCCGCGCCCACTTTCCACTGGA
TGTGCAGTGGAATGACCTGGATTACATGGACTCCAGGAGAGACTTCACCTTCAACAAGGACG
GCTTCAGGGATTTTCCCGCCATGGTGCAGGAGCTGCACCAGGGCGGCCGGCGCTACATGATGA
TCGTGGACCCCGCCATCTCCTCTAGCGGACCTGCCGGCAGCTACAGACCATATGACGAGGGCC
TGAGGAGAGGCGTGTTCATCACAAACGAGACCGGCCAGCCTCTGATCGGCAAGGTCTGGCCA
GGCTCCACCGCCTTCCCAGACTTCACCAATCCAACCGCCCTGGCCTGGTGGGAGGACATGGTG
GCCGAGTTCCACGACCAGGTGCCTTTTGATGGCATGTGGATCGACATGAACGAGCCATCTAA
TTTCATCAGGGGCAGCGAGGACGGCTGCCCCAACAATGAGCTGGAGAACCCACCATATGTGCC
TGGCGTGGTGGGAGGCACCCTCCAGGCAGCAACCATCTGTGCCTCCTCTCACCAGTTTCTGTC
TACACACTATAACCTGCACAATCTGTACGGACTGACCGAGGCAATCGCCAGCCACAGAGCCCT
GGTGAAGGCCAGGGGCACAAGACCTTTCGTGATCTCCAGGTCTACCTTTGCCGGACACGGCAG
ATACGCAGGACACTGGACCGGCGACGTGTGGAGCAGCTGGGAGCAGCTGGCCTCTAGCGTGCC
AGAGATCCTCCAGTTCAACCTGCTGGGCGTGCCCCTGGTGGGAGCAGACGTGTGCGGCTTTCT
GGGCAATACATCCGAGGAGCTGTGCGTGAGGTGGACCCAGCTGGGAGCCTTCTATCCCTTCAT
GCGCAACCACAATAGCCTGCTGTCCCTGCCTCAGGAGCCATACAGCTTCTCCGAGCCTGCACA
GCAGGCAATGAGGAAGGCCCTGACACTGCGCTATGCCCTGCTGCCACACCTGTACACCCTGTT
TCACCAGGCACACGTGGCAGGAGAGACAGTGGCCCGGCCCCTGTTCCTGGAGTTTCCTAAGGA
TTCCTCTACCTGGACAGTGGACCACCAGCTGCTGTGGGGAGAGGCCCTGCTGATCACCCCCGT
GCTCCAGGCAGGCAAGGCAGAGGTGACAGGCTATTTCCCTCTGGGCACATGGTACGACCTCCA
GACCGTGCCAGTGGAGGCCCTGGGCAGCCTGCCTCCACCACCTGCCGCCCCCCGCGAGCCTGCC
ATCCACTCCGAGGGACAGTGGGTGACACTGCCAGCACCTCTGGACACCATCAACGTGCACCTG
AGGGCCGGCTATATCATCCCCCTCCAGGGCCCTGGCCTGACCACAACCGAGTCCAGACAGCAG
CCAATGGCCCTGGCCGTGGCCCTGACCAAGGGAGGCGAGGCCAGGGGCGAGCTGTTCTGGGAC
GATGGCGAGTCTCTGGAGGTGCTGGAGAGAGGCGCCTACACACAGGTCATCTTCCTGGCCAG
GAACAATACAATCGTGAATGAGCTGGTGAGAGTGACCTCTGAGGGAGCAGGACTCCAGCTCC
AGAAGGTGACAGTGCTGGGAGTGGCAACCGCACCACAGCAGGTGCTGAGCAACGGCGTGCCC
GTGAGCAATTTCACATACTCCCCTGATACCAAGGTGCTGGACATCTGCGTGAGCCTGCTGATG
GGCGAGCAGTTTCTGGTGTCCTGGTGTTGA
(Dual promoter vector)
>pDY-DP
SEQ ID NO: 10
AAATTGTAAGCGTTAATATTTTGTTAAAATTCGCGTTAAATTTTTGTTAAATCAGCTCATTTTTTAACCAATAGG
CCGAAATCGGCAAAATCCCTTATAAATCAAAAGAATAGACCGAGATAGGGTTGAGTGTTGTTCCAGTTTGGAA
CAAGAGTCCACTATTAAAGAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCACTAAATCGGAACCCTAAA
GGGAGCCCCCGATTTAGAGCTTGACGGGGAAAGCCGGCGAACGTGGCGAGAAAGGAAGGGAAGAAAGCGA
AAGGAGCGGGCGCTAGGGCGCTGGCAAGTGTAGCGGTCACGCTGCGCGTAACCACCACACCCGCCGCGCTT
AATGCGCCGCTACAGGGCGCGTCCATTCGCCATTCAGGCTGCGCAACTGTTGGGAAGGGCGATCGGTGCGGG
CCTCTTCGCTATTACGCCAGCTGGCGAAAGGGGGATGTGCTGCAAGGCGATTAAGTTGGGTAACGCCAGGGT
TTTCCCAGTCACGACGTTGTAAAACGACGGCCAGTGAATTGTAATACGACTCACTATAGGGCGAATTGGGCCC
GACGTCGCATGCTTGGAAGGGCTAATTCACTCCCAAAGAAGACAAGATATCCTTGATCTGTGGATCTACCACA
CACAAGGCTACTTCCCTGATTAGCAGAACTACACACCAGGGCCAGGGGTCAGATATCCACTGACCTTTGGATG
GTGCTACAAGCTAGTACCAGTTGAGCCAGATAAGGTAGAAGAGGCCAATAAAGGAGAGAACACCAGCTTGTT
ACACCCTGTGAGCCTGCATGGGATGGATGACCCGGAGAGAGAAGTGTTAGAGTGGAGGTTTGACAGCCGCC
TAGCATTTCATCACGTGGCCCGAGAGCTGCATCCGGAGTACTTCAAGAACTGCTGATATCGAGCTTGCTACAA
GGGACTTTCCGCTGGGGACTTTCCAGGGAGGCGTGGCCTGGGCGGGACTGGGGAGTGGCGAGCCCTCAGAT
CCTGCATATAAGCAGCTGCTTTTTGCCTGTACTGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTTCAAGTAGTGTGTGCCCGT
CTGTTGTGTGACTCTGGTAACTAGAGATCCCTCAGACCCTTTTAGTCAGTGTGGAAAATCTCTAGCAGTGGCGC
CCGAACAGGGACTTGAAAGCGAAAGGGAAACCAGAGGAGCTCTCTCGACGCAGGACTCGGCTTGCTGAAGC
GCGCACGGCAAGAGGCGAGGGGCGGCGACTGGTGAGTACGCCAAAAATTTTGACTAGCGGAGGCTAGAAG
GAGAGAGATGGGTGCGAGAGCGTCAGTATTAAGCGGGGGAGAATTAGATCGCGATGGGAAAAAATTCGGTT
AAGGCCAGGGGGAAAGAAAAAATATAAATTAAAACATATAGTATGGGCAAGCAGGGAGCTAGAACGATTCG
CAGTTAATCCTGGCCTGTTAGAAACATCAGAAGGCTGTAGACAAATACTGGGACAGCTACAACCATCCCTTCA
GACAGGATCAGAAGAACTTAGATCATTATATAATAGAGTAGCAACCCTCTATTGTGTGCATCAAAGGATAGAG
ATAAAAGACACCAAGGAAGCTTTAGACAAGATAGAGGAAGAGCAAAACAAAAGTAAGACCACCGCACAGCA
AGCGGCCGCTGATCTTCAGACCTGGAGGAGGAGATATGAGGGACAATTGGAGAAGTGAATTATATAAATATA
AAGTAGTAAAAATTGAACCATTAGGAGTAGCACCCACCAAGGCAAAGAGAAGAGTGGTGCAGAGAGAAAAA
AGAGCAGTGGGAATAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAATTTGCTGAGGGCT
ATTGAGGCGCAACAGCATCTGTTGCAACTCACAGTCTGGGGCATCAAGCAGCTCCAGGCAAGAATCCTGGCT
GTGGAAAGATACCTAAAGGATCAACAGCTCCTGGGGATTTGGGGTTGCTCTGGAAAACTCATTTGCACCACTG
CTGTGCCTTGGAATGCTAGTTGGAGTAATAAATCTCTGGAACAGATTTGGAATCACACGACCTGGATGGAGTG
GGACAGAGAAATTAACAATTACACAAGCTTAATACACTCCTTAATTGAAGAATCGCAAAACCAGCAAGAAAAG
AATGAACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTTCTATA
GTGAATAGAGTTAGGCAGGGATATTCACCATTATCGTTTCAGACCCACCTCCCAACCCCGAGGGGACCCGACA
GGCCCGAAGGAATAGAAGAAGAAGGTGGAGAGAGAGACAGAGACAGATCCATTCGATTAGTGAACGGATCT
CGACGGGATCGATTTTAAAAGAAAAGGGGGGATTGGGGGGTACAGTGCAGGGGAAAGAATAGTAGACATA
ATAGCAACAGACATACAAACTAAAGAATTACAAAAACAAATTACAAAAATTCAAAATTTTATCGATAAGCTTTG
CAAAGATGGATAAAGTTTTAAACAGAGAGGAATCTTTGCAGCTAATGGACCTTCTAGGTCTGACCCCGTACGC
CTCGAGAGATCTGATCATAATCAGCCATACCACATTTGTAGAGGTTTTACTTGCTTTAAAAAACCTCCCACACCT
CCCCCTGAACCTGAAACATAAAATGAATGCAATTGTTGTTGTTAACTTGTTTATTGCAGCTTATAATGGTTACA
AATAAGGCAATAGCATCACAAATTTCACAAATAAGGCATTTTTTTCACTGCATTCTAGTTTTGGTTTGTCCAAAC
TCATCAATGTATCTTATCATGTCTGGATCTCAAATCCCTCGGAAGCTGCGCCTGTCTTAGGTTGGAGTGATACA
TTTTTATCACTTTTACCCGTCTTTGGATTAGGCAGTAGCTCTGACGGCCCTCCTGTCTTAGGTTAGTGAAAAATG
TCACTCTCTTACCCGTCATTGGCTGTCCAGCTTAGCTCGCAGGGGAGGTGGTCTGCCTGCAGGCGGATGGCGT
TAACATATGACAACTTTCTCCCGGGTAATCTGACCGTTCGCTAGCCCTGGGGAGAGAGGTCGGTGATTCGGTC
AACGAGGGAGCCGACTGCCGACGTGCGCTCCGGAGGCTTGCAGAATGCGGAACACCGCGCGGGCAGGAACA
GGGCCCACACTACCGCCCCACACCCCGCCTCCCGCACCGCCCCTTCCCGGCCGCTGCTCTCGGCGCGCCCCGCT
GAGCAGCCGCTATTGGCCACAGCCCATCGCGGTCGGCGCGCTGCCATTGCTCCCTGGCGCTGTCCGTCTGCGA
GGGTACTAGTGAGACGTGCGGCTTCCGTTTGTCACGTCCGGCACGCCGCGAACCGCAAGGAACCTTCCCGACT
TAGGGGCGGAGCAGGAAGCGTCGCCGGGGGGCCCACAAGGGTAGCGGCGAAGATCCGGGTGACGCTGCGA
ACGGACGTGAAGAATGTGCGAGACCCAGGGTCGGCGCCGCTGCGTTTCCCGGAACCACGCCCAGAGCAGCC
GCGTCCCTGCGCAAACCCAGGGCTGCCAAGGAAAAGGCGCAACCCCAACCCCGTGGTTAATTAAGGTGAAAG
GAGTGGGAATTGGCTCCGGTGCCCGTCAGTGGGCAGAGCGCACATCGCCCACAGTCCCCGAGAAGTTGGGG
GGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTT
TCGCAACGGGTTTGCCGCCAGAACACAGGTAAGTGCCGTGTGTGGTTCCCGCGGGCCTGGCCTCTTTACGGG
TTATGGCCCTTGCGTGCCTTGAATTACTTCCACCTGGCTGCAGTACGTGATTCTTGATCCCGAGCTTCGGGTTG
GAAGTGGGTGGGAGAGTTCGAGGCCTTGCGCTTAAGGAGCCCCTTCGCCTCGTGCTTGAGTTGAGGCCTGGC
CTGGGCGCTGGGGCCGCCGCGTGCGAATCTGGTGGCACCTTCGCGCCTGTCTCGCTGCTTTCGATAAGTCTCT
AGCCATTTAAAATTTTTGATGACCTGCTGCGACGCTTTTTTTCTGGCAAGATAGTCTTGTAAATGCGGGCCAAG
ATCTGCACACTGGTATTTCGGTTTTTGGGGCCGCGGGCGGCGACGGGGCCCGTGCGTCCCAGCGCACATGTT
CGGCGAGGCGGGGCCTGCGAGCGCGGCCACCGAGAATCGGACGGGGGTAGTCTCAAGCTGGCCGGCCTGCT
CTGGTGCCTGGCCTCGCGCCGCCGTGTATCGCCCCGCCCTGGGCGGCAAGGCTGGCCCGGTCGGCACCAGTT
GCGTGAGCGGAAAGATGGCCGCTTCCCGGCCCTGCTGCAGGGAGCTCAAAATGGAGGACGCGGCGCTCGGG
AGAGCGGGCGGGTGAGTCACCCACACAAAGGAAAAGGGCCTTTCCGTCCTCAGCCGTCGCTTCATGTGACTC
CACGGAGTACCGGGCGCCGTCCAGGCACCTCGATTAGTTCTCGAGCTTTTGGAGTACGTCGTCTTTAGGTTGG
GGGGAGGGGTTTTATGCGATGGAGTTTCCCCACACTGAGTGGGTGGAGACTGAAGTTAGGCCAGCTTGGCAC
TTGATGTAATTCTCCTTGGAATTTGCCCTTTTTGAGTTTGGATCTTGGTTCATTCTCAAGCCTCAGACAGTGGTT
CAAAGTTTTTTTCTTCCATTTCAGGTGTCGTGAGGAATTCTGCAGTCGACGGTACCGCGGGCGCGCCCCGGGA
TCCAAGCTTCAATTGTGGTCACTCGACAATCAACCTCTGGATTACAAAATTTGTGAAAGATTGACTGGTATTCT
TAACTATGTTGCTCCTTTTACGCTATGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAGGAGTTGTGGCCCGTTGTCAGGCAA
CGTGGCGTGGTGTGCACTGTGTTTGCTGACGCAACCCCCACTGGTTGGGGCATTGCCACCACCGTCAGCTCC
TTTCCGGGACTTTCGCTTTCCCCCTCCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGAAGCTGACGTCCTTTCCATGGCTG
CTCGCCTGTGTTGCCACCTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGCCCTCAGACGAGTCGGA
TCTCCCTTTGGGCCGCCTCCCCGCCTGCTCGAGACCTAGAAAAACATGGAGCAATCACAAGTAGCAATACAGC
AGCTACCAATGCTGATTGTGCCTGGCTAGAAGCACAAGAGGAGGAGGAGGTGGGTTTTCCAGTCACACCTCA
GGTACCTTTAAGACCAATGACTTACAAGGCAGCTGTAGATCTTAGCCACTTTTTAAAAGAAAAGGGGGGACTG
GAAGGGCTAATTCACTCCCAACGAAGACAAGATCTGCTTTTTGCTTGTACTGGGTCTCTCTGGTTAGACCAGAT
CTGAGCCTGGGAGCTCTCTGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGACCCTTTTAGTCAGTGTGGA
AAATCTCTAGCAGTAGTAGTTCATGTCATCTTATTATTCAGTATTTATAACTTGCAAAGAAATGAATATCAGAG
AGTGAGAGGACGCGTTGGATGCATAGCTTGAGTATTCTATAGTGTCACCTAAATAGCTTGGCGTAATCATGGT
CATAGCTGTTTCCTGTGTGAAATTGTTATCCGCTCACAATTCCACACAACATACGAGCCGGAAGCATAAAGTGT
AAAGCCTGGGGTGCCTAATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGGTATCAGCTCACTCAAAG
GCGGTAATACGGTTATCCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCCCCTGGAAGC
TCCCTCGTGCGCTCTCCTGTTCCGACCCTGCCGCTTACCGGATACCTGTCCGCCTTTCTCCCTTCGGGAAGCGTG
GCGCTTTCTCATAGCTCACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTAGCGGTGGTTTTTTTGTT
TGCAAGCAGCAGATTACGCGCAGAAAAAAAGGATCTCAAGAAGATCCTTTGATCTTTTCTACGGGGTCTGACG
CTCAGTGGAACGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAGTTACCAATGCT
TAATCAGTGAGGCACCTATCTCAGCGATCTGTCTATTTCGTTCATCCATAGTTGCCTGACTCCCCGTCGTGTAG
ATAACTACGATACGGGAGGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATCGTTGTC
AGAAGTAAGTTGGCCGCAGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTCGATGTAACCCACTCGTGC
ACCCAACTGATCTTCAGCATCTTTTACTTTCACCAGCGTTTCTGGGTGAGCAAAAACAGGAAGGCAAAATGCC
GCAAAAAAGGGAATAAGGGCGACACGGAAATGTTGAATACTCATACTCTTCCTTTTTCAATATTATTGAAGCA
TTTATCAGGGTTATTGTCTCATGAGCGGATACATATTTGAATGTATTTAGAAAAATAAACAAATAGGGGTTCCG
CGCACATTTCCCCGAAAAGTGCCACCTGATGCGGTGTGAAATACCGCACAGATGCGTAAGGAGAAAATACCG
CATCAGG
>pDY-[MCS] + (IY)
SEQ ID NO: 11
AAATTGTAAGCGTTAATATTTTGTTAAAATTCGCGTTAAATTTTTGTTAAATCAGCTCATTTTTTAACCAATAGG
CCGAAATCGGCAAAATCCCTTATAAATCAAAAGAATAGACCGAGATAGGGTTGAGTGTTGTTCCAGTTTGGAA
CAAGAGTCCACTATTAAAGAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCACTAAATCGGAACCCTAAA
GGGAGCCCCCGATTTAGAGCTTGACGGGGAAAGCCGGCGAACGTGGCGAGAAAGGAAGGGAAGAAAGCGA
AAGGAGCGGGCGCTAGGGCGCTGGCAAGTGTAGCGGTCACGCTGCGCGTAACCACCACACCCGCCGCGCTT
AATGCGCCGCTACAGGGCGCGTCCATTCGCCATTCAGGCTGCGCAACTGTTGGGAAGGGCGATCGGTGCGGG
CCTCTTCGCTATTACGCCAGCTGGCGAAAGGGGGATGTGCTGCAAGGCGATTAAGTTGGGTAACGCCAGGGT
TTTCCCAGTCACGACGTTGTAAAACGACGGCCAGTGAATTGTAATACGACTCACTATAGGGCGAATTGGGCCC
GACGTCGCATGCTTGGAAGGGCTAATTCACTCCCAAAGAAGACAAGATATCCTTGATCTGTGGATCTACCACA
CACAAGGCTACTTCCCTGATTAGCAGAACTACACACCAGGGCCAGGGGTCAGATATCCACTGACCTTTGGATG
GTGCTACAAGCTAGTACCAGTTGAGCCAGATAAGGTAGAAGAGGCCAATAAAGGAGAGAACACCAGCTTGTT
ACACCCTGTGAGCCTGCATGGGATGGATGACCCGGAGAGAGAAGTGTTAGAGTGGAGGTTTGACAGCCGCC
TAGCATTTCATCACGTGGCCCGAGAGCTGCATCCGGAGTACTTCAAGAACTGCTGATATCGAGCTTGCTACAA
GGGACTTTCCGCTGGGGACTTTCCAGGGAGGCGTGGCCTGGGCGGGACTGGGGAGTGGCGAGCCCTCAGAT
CCTGCATATAAGCAGCTGCTTTTTGCCTGTACTGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTTCAAGTAGTGTGTGCCCGT
CTGTTGTGTGACTCTGGTAACTAGAGATCCCTCAGACCCTTTTAGTCAGTGTGGAAAATCTCTAGCAGTGGCGC
CCGAACAGGGACTTGAAAGCGAAAGGGAAACCAGAGGAGCTCTCTCGACGCAGGACTCGGCTTGCTGAAGC
GCGCACGGCAAGAGGCGAGGGGCGGCGACTGGTGAGTACGCCAAAAATTTTGACTAGCGGAGGCTAGAAG
GAGAGAGATGGGTGCGAGAGCGTCAGTATTAAGCGGGGGAGAATTAGATCGCGATGGGAAAAAATTCGGTT
AAGGCCAGGGGGAAAGAAAAAATATAAATTAAAACATATAGTATGGGCAAGCAGGGAGCTAGAACGATTCG
CAGTTAATCCTGGCCTGTTAGAAACATCAGAAGGCTGTAGACAAATACTGGGACAGCTACAACCATCCCTTCA
GACAGGATCAGAAGAACTTAGATCATTATATAATACAGTAGCAACCCTCTATTGTGTGCATCAAAGGATAGAG
ATAAAAGACACCAAGGAAGCTTTAGACAAGATAGAGGAAGAGCAAAACAAAAGTAAGACCACCGCACAGCA
AGCGGCCGCTGATCTTCAGACCTGGAGGAGGAGATATGAGGGACAATTGGAGAAGTGAATTATATAAATATA
AAGTAGTAAAAATTGAACCATTAGGAGTAGCACCCACCAAGGCAAAGAGAAGAGTGGTGCAGAGAGAAAAA
AGAGCAGTGGGAATAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAATTTGCTGAGGGCT
ATTGAGGCGCAACAGCATCTGTTGCAACTCACAGTCTGGGGCATCAAGCAGCTCCAGGCAAGAATCCTGGCT
GTGGAAAGATACCTAAAGGATCAACAGCTCCTGGGGATTTGGGGTTGCTCTGGAAAACTCATTTGCACCACTG
CTGTGCCTTGGAATGCTAGTTGGAGTAATAAATCTCTGGAACAGATTTGGAATCACACGACCTGGATGGAGTG
GGACAGAGAAATTAACAATTACACAAGCTTAATACACTCCTTAATTGAAGAATCGCAAAACCAGCAAGAAAAG
AATGAACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTTCTATA
GTGAATAGAGTTAGGCAGGGATATTCACCATTATCGTTTCAGACCCACCTCCCAACCCCGAGGGGACCCGACA
GGCCCGAAGGAATAGAAGAAGAAGGTGGAGAGAGAGACAGAGACAGATCCATTCGATTAGTGAACGGATCT
CGACGGGATCGATTTTAAAAGAAAAGGGGGGATTGGGGGGTACAGTGCAGGGGAAAGAATAGTAGACATA
ATAGCAACAGACATACAAACTAAAGAATTACAAAAACAAATTACAAAAATTCAAAATTTTATCGATAAGCTTTG
CAAAGATGGATAAAGTTTTAAACAGAGAGGAATCTTTGCAGCTAATGGACCTTCTAGGTCTGACCCCGTACGC
CTCGAGAGATCTGATCATAATCAGCCATACCACATTTGTAGAGGTTTTACTTGCTTTAAAAAACCTCCCACACCT
CCCCCTGAACCTGAAACATAAAATGAATGCAATTGTTGTTGTTAACTTGTTTATTGCAGCTTATAATGGTTACA
AATAAGGCAATAGCATCACAAATTTCACAAATAAGGCATTTTTTTCACTGCATTCTAGTTTTGGTTTGTCCAAAC
TCATCAATGTATCTTATCATGTCTGGATCTCAAATCCCTCGGAAGCTGCGCCTGTCTTAGGTTGGAGTGATACA
TTTTTATCACTTTTACCCGTCTTTGGATTAGGCAGTAGCTCTGACGGCCCTCCTGTCTTAGGTTAGTGAAAAATG
TCACTCTCTTACCCGTCATTGGCTGTCCAGCTTAGCTCGCAGGGGAGGTGGTCTGCCTGCAGGTTAGAACAGT
CTCTTTTCGTATGAGTGCAGTGAGTGGACGCCGCCTTCGACCTGGGCTGAAGAAGTTCTTTTCTCGAACTTCAG
TTCGCCGGAATACATCATTGCCCGCACCTGTGTCAGGCTCTTAGCGCCGATATCCTGGCATGAATGCTGAATTC
CGGCGATCAGGTAAGGCACGAATTTGTGAATACTGCCCTTATCCTGGACAGCTCCAGACACGCCCTGTGCGAC
TTTGATCTTGTCTGCCTCGGAAAAATACCTGTTCTGAGAGGACAGATGCTTATCCATGGCGTCCAGTGACCCCA
TGCCCCTATATTTCTTCAGTCTGAACCCATCACTAAAGAAGTACTCGCCGGGGGCTTCTGTGGTTGCAGCCAGC
AGGCTGCCCATCATCACTGTGCTTGCCCCCAGAGCCAGGGCTTTTGCGATGTGGCCCACATTCTGAATTCCCCC
GTCAGCGATCACTGGGACTCCGAATCTCCGGGCATACTCGTACACCTTGTAGACAGCAGTTGCCTGAGGTCGT
CCACAGGCCAGCACTTCCTGAATGATGCAGATTGATCCACTCCCCATTCCGACCCTCAGAGCATCCACTCCTGC
GTCAATCAGGTTTTTGGCCTGGGCTGCGGTCACGACATTGCCTCCGATGACCTGCAGATTTGGGTACTTGTCCT
TAATGTACTTGATCATATTAATCTGGAAGATGCTGTTTCCCTGGCTTGAATCCAGCACGACCACGTCCACCCCT
GCCTGAGCCAGCAGATCCAGGCGATATTTATCGTCCTCGTGTGTGCCAATAGCGGCTCCACACAGCAGCTGTT
TCTTTGCGTCCTTACTAGCCAGAGGGTAATCTCGATTTTTCTTCAGGTCGGTGCGGGCAATGATTGCCACCAGC
TCATCGTCTTCATTCACGATAGGCAGTTTTCCTTTCTTAGACCGCTGCAGAATCTCGTTGGCTTCCTTCAGTGTG
ATGCCGGCAGGTGCGACCACCAGATCTTCGCGTTTGGTCATAATCTCTTCCAGAAAACAGTCATGCTCTTCCTC
CTTCAGGAAATCGATGTCTCGACTAGAAATGATTCCTCACCAGTCGGCTGCCCATTCGTCCAGTATCTGTAATGG
GGATGCCGCAAAATCCGTGCCTAGCTTTGGCCTCGAACACATCGCGGACCCTGTCCTTGGGGCTCAGGACCAC
TGGGTCGGTGATAAAGCCCTGTTCGTATTTCTTCACCTTTCTGACCTCATTGGCCTGAAATTCTGGAGTGCAGT
TATGGTGAATGAACCCGATCCCGCCTGTCAGTGCCATAGCAATGGCCATGCCAGCCTCGGTGACAGTGTCCAT
AGGGGAGCTCACCAGGGGTGTCTTCAGGGTGATTTTCTTGGTCAGGGCAGAAGTCAGATCCACCTGGTCTGC
GGTAAAATCAATATAGCCGGGCAGGATCAGGAAGTCGTTGTAAGTCAGCCCGTCTCCACAATTAAACAGCTG
CTGGGCGGTCAGTCCATCATCAGGGACATAGGAAGTGCCTCCAGAAATCAGGTAGTCGGCCATGGTGGCGCT
AGCCCTGGGGAGAGAGGTCGGTGATTCGGTCAACGAGGGAGCCGACTGCCGACGTGCGCTCCGGAGGCTTG
CAGAATGCGGAACACCGCGCGGGCAGGAACAGGGCCCACACTACCGCCCCTCACCCCGCCTCCCGCACCGCC
CCTTCCCGGCCGCTGCTCTCGGCGCGCCCCGCTGAGCAGCCGCTATTGGCCACAGCCCATCGCGGTCGGCGCG
CTGCCATTGCTCCCTGGCGCTGTCCGTCTGCGAGGGTACTAGTGAGACGTGCGGCTTCCGTTTGTCACGTCCG
GCACGCCGCGAACCGCAAGGAACCTTCCCGACTTAGGGGCGGAGCAGGAAGCGTCGCCGGGGGGCCCACAA
GGGTAGCGGCGAAGATCCGGGTGACGCTGCGAACGGACGTGAAGAATGTGCGAGACCCAGGGTCGGCGCC
GCTGCGTTTCCCGGAACCACGCCCAGAGCAGCCGCGTCCCTGCGCAAACCCAGGGCTGCCAAGGAAAAGGCG
CAACCCCAACCCCGTGGTTAATTAAGGTGAAAGGAGTGGGAATTGGCTCCGGTGCCCGTCAGTGGGCAGAGC
GCACATCGCCCACAGTCCCCGAGAAGTTGGGGGGAGGGGTCGGCAATTGAACCGGTGCCTAGAGAAGGTGG
CGCGGGGTAAACTGGGAAAGTGATGTCGTGTACTGGCTCCGCCTTTTTCCCGAGGGTGGGGGAGAACCGTAT
ATAAGTGCAGTAGTCGCCGTGAACGTTCTTTTTCGCAACGGGTTTGCCGCCAGAACACAGGTAAGTGCCGTGT
GTGGTTCCCGCGGGCCTGGCCTCTTTACGGGTTATGGCCCTTGCGTGCCTTGAATTACTTCCACCTGGCTGCAG
TACGTGATTCTTGATCCCGAGCTTCGGGTTGGAAGTGGGTGGGAGAGTTCGAGGCCTTGCGCTTAAGGAGCC
CCTTCGCCTCGTGCTTGAGTTGAGGCCTGGCCTGGGCGCTGGGGCCGCCGCGTGCGAATCTGGTGGCACCTTC
GCGCCTGTCTCGCTGCTTTCGATAAGTCTCTAGCCATTTAAAATTTTTGATGACCTGCTGCGACGGTTTTTTTCT
GGCAAGATAGTCTTGTAAATGCGGGCCAAGATCTGCACACTGGTATTTCGGTTTTTGGGGCCGCGGGCGGCG
ACGGGGCCCGTGCGTCCCAGCGCACATGTTCGGCGAGGCGGGGCCTGCGAGCGCGGCCACCGAGAATCGGA
CGGGGGTAGTCTCAAGCTGGCCGGCCTGCTCTGGTGCCTGGCCTCGCGCCGCCGTGTATCGCCCCGCCCTGG
GCGGCAAGGCTGGCCCGGTCGGCACCAGTTGCGTGAGCGGAAAGATGGCCGCTTCCCGGCCCTGCTGCAGG
GAGCTCAAAATGGAGGACGCGGCGCTCGGGAGAGCGGGCGGGTGAGTCACCCACACAAAGGAAAAGGGCC
TTTCCGTCCTCAGCCGTCGCTTCATGTGACTCCACGGAGTACCGGGCGCCGTCCAGGCACCTCGATTAGTTCTC
GAGCTTTTGGAGTACGTCGTCTTTAGGTTGGGGGGAGGGGTTTTATGCGATGGAGTTTCCCCACACTGAGTG
GGTGGAGACTGAAGTTAGGCCAGCTTGGCACTTGATGTAATTCTCCTTGGAATTTGCCCTTTTTGAGTTTGGAT
AGTCGACGGTACCGCGGGCGCGCCCCGGGATCCAAGCTTCAATTGTGGTCACTCGACAATCAACCTCTGGATT
ACAAAATTTGTGAAAGATTGACTGGTATTCTTAACTATGTTGCTCCTTTTACGCTATGTGGATACGCTGCTTTAA
TGCCTTTGTATCATGCTATTGCTTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTGCTGACGCAACCCCCACTG
GTTGGGGCATTGCCACCACCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTGACAATTCCGTGGTGTTGT
CGGGGAAGCTGACGTCCTTTCCATGGCTGCTCGCCTGTGTTGCCACCTGGATTCTGCGCGGGACGTCCTTCTG
CTACGTCCCTTCGGCCCTCAATCCAGCGGACCTTCCTTCCCGCGGCCTGCTGCCGGCTCTGCGGCCTCTTCCGC
GTCTTCGCCTTCGCCCTCAGACGAGTCGGATCTCCCTTTGGGCCGCCTCCCCGCCTGCTCGAGACCTAGAAAAA
CATGGAGCAATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCTGTAGATCTTA
GCCACTTTTTAAAAGAAAAGGGGGGACTGGAAGGGCTAATTCACTCCCAACGAAGACAAGATCTGCTTTTTGC
TTGTACTGGGTCTCTCTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGACTCTGGTAACTAGA
GATCCCTCAGACCCTTTTAGTCAGTGTGGAAAATCTCTAGCAGTAGTAGTTCATGTCATCTTATTATTCAGTATT
TATAACTTGCAAAGAAATGAATATCAGAGAGTGAGAGGACGCGTTGGATGCATAGCTTGAGTATTCTATAGT
GTCACCTAAATAGCTTGGCGTAATCATGGTCATAGCTGTTTCCTGTGTGAAATTGTTATCCGCTCACAATTCCAC
ACAACATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCAACGCGC
GGGGAGAGGCGGTTTGCGTATTGGGCGCTCTTCCGCTTCCTCGCTCACTGACTCGCTGCGCTCGGTCGTTCGG
CTGCGGCGAGCGGTATCAGCTCACTCAAAGGGGGTAATACGGTTATCCACAGAATCAGGGGATAACGCAGGA
AAGAACATGTGAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCCGACAGGACTA
TAAAGATACCAGGCGTTTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGGTATCTCAGTTCGGTGT
AGGTCGTTCGCTCCAAGCTGGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTGATCCGGCAAAC
AAACCACCGCTGGTAGCGGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAATCAATCTAAAGTATAT
ATGAGTAAACTTGGTCTGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTACCATCTGGCCCCAGTG
CTGCAATGATACCGCGAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGCCGGGAAGCTAGAGT
AAGTAGTTCGCCAGTTAATAGTTTGCGCAACGTTGTTGCCATTGCTACAGGCATCGTGGTGTCACGCTCGTCGT
TTGGTATGGCTTCATTCAGCTCCGGTTCCCAACGATCAAGGCGAGTTACATGATCCCCCATGTTGTGCAAAAAA
GCGGTTAGCTCCTTCGGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTGAGTACTCAACCAAGT
CATTCTGAGAATAGTGTATGCGGCGACCGAGTTGCTCTTGCCCGGCGTCAATACGGGATAATACCGCGCCACA
TAGCAGAACTTTTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTGATGCGGTGTGAAA
TACCGCACAGATGCGTAAGGAGAAAATACCGCATCAGG

1. A method of producing a population of transduced T-Rapa cells that express an enzyme encoded by a transgene of interest for the treatment of a lysosomal storage disorder, the method comprising:
(a) conditioning T-cells from a subject or suitable donor with rapamycin ex vivo to generate T-Rapa cells; and
(b) transducing the T-Rapa cells in vitro with a vector comprising the transgene of interest to produce a population of transduced T-Rapa cells.

2. The method of claim 1, wherein the method further comprises:
(c) expanding the transduced T-Rapa cells in in vitro culture.

3. The method of claim 2, wherein a portion of the expanded transduced T-Rapa cells are cryopreserved for future use.

4. The method of claim 2, further comprising detecting and isolating CD4+ T-cells from a subject or suitable donor and culturing the CD4+ T-cells in vitro prior to step (a), wherein the purity of the isolated CD4+ T cells is at least 75%.

5. The method of claim 1, wherein step (a) comprises culturing the T-cells in chemically defined medium comprising about 0.1 to about 2 micromolar rapamycin.

6. The method of claim 5, wherein the chemically defined medium further comprises recombinant human IL-4 and recombinant human IL-2.

7. The method of claim 1, wherein the vector is a lentiviral vector.

8. The method of claim 1, wherein the transgene is selected from the group consisting of AGA, GBA, ASAH1, and GAA.

9. A transduced T-Rapa cell population made by the method of claim 1.


SEQUENCE LISTING (11 sequences)

SEQ ID NO: 1 - DNA, 1290 bp, synthetic construct
  misc_feature 1..1290: note = AGA transgene
  source 1..1290: mol_type = other DNA
atgcaacttcgaaacccagagctccacctcggatgtgcccttgctctgaggttcctggcgctggtgtcttgggatatacccggagcacgcgctctggacaacgggctggcccggactccaaccatgggttggctccattgggaaaggtttatgtgcaacttggactgccaggaagaacccgactcctgtatttccgagaaactcttcatggagatggccgagctgatggttagcgaaggctggaaggatgccggttatgaatacttgtgtatcgacgattgttggatggctccccagcgggacagtgaaggacgactccaggcagatccgcaacggttccctcatggcatacggcagctcgccaattacgtgcacagcaagggtttgaagctggggatatatgctgacgtgggcaacaaaacctgtgctggtttccccggcagcttcggctactatgatatagatgcacaaaccttcgctgattggggcgtggacctgcttaaatttgacggctgttactgcgacagcttggaaaacctcgccgatggatataaacacatgagccttgcactcaatcggactggccggagcattgtctactcttgcgagtggccattgtacatgtggcctttccagaagcctaactatacggagattagacagtattgtaatcactggagaaactttgcagatatcgacgactcatggaagtccatcaaatctattctggactggacttcattcaatcaggagcgcatcgtcgatgttgccggtccaggtggatggaacgaccctgacatgctcgtaattgggaatttcggactgtcctggaatcagcaggtcacacagatggctttgtgggctatcatggcagccccactctttatgtctaacgatttgcggcatatttcaccacaggccaaagccctgctgcaagataaggacgtcatagcgattaaccaggacccactgggaaagcagggctaccagctgagacagggcgacaattttgaggtctgggaaagacctcttagcgggctggcgtgggccgtagccatgattaatcgccaggaaattggcggccctcgctcttacactatcgcggtcgccagtctgggcaagggagtcgcttgtaaccccgcctgcttcataactcagttgctgcccgtgaaacggaagctgggcttctatgaatggactagcagactccgcagtcatattaatccgactggtacggtgctgctgcaactggagaataccatgcagatgtcacttaaggatcttctgtga

SEQ ID NO: 2 - DNA, 8767 bp, synthetic construct
  misc_feature 1..8767: note = Lentiviral vector comprising AGA transgene
  source 1..8767: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgccaccatgcaacttcgaaacccagagctccacctcggatgtgcccttgctctgaggttcctggcgctggtgtcttgggatatacccggagcacgcgctctggacaacgggctggcccggactccaaccatgggttggctccattgggaaaggtttatgtgcaacttggactgccaggaagaacccgactcctgtatttccgagaaactcttcatggagatggccgagctgatggttagcgaaggctggaaggatgccggttatgaatacttgtgtatcgacgattgttggatggctccccagcgggacagtgaaggacgactccaggcagatccgcaacggttccctcatggcatacggcagctcgccaattacgtgcacagcaagggtttgaagctggggatatatgctgacgtgggcaacaaaacctgtgctggtttccccggcagcttcggctactatgatatagatgcacaaaccttcgctgattggggcgtggacctgcttaaatttgacggctgttactgcgacagcttggaaaacctcgccgatggatataaacacatgagccttgcactcaatcggactggccggagcattgtctactcttgcgagtggccattgtacatgtggcctttccagaagcctaactatacggagattagacagtattgtaatcactggagaaactttgcagatatcgacgactcatggaagtccatcaaatctattctggactggacttcattcaatcaggagcgcatcgtcgatgttgccggtccaggtggatggaacgaccctgacatgctcgtaattgggaatttcggactgtcctggaatcagcaggtcacacagatggctttgtgggctatcatggcagccccactctttatgtctaacgatttgcggcatatttcaccacaggccaaagccctgctgcaagataaggacgtcatagcgattaaccaggacccactgggaaagcagggctaccagctgagacagggcgacaattttgaggtctgggaaagacctcttagcgggctggcgtgggccgtagccatgattaatcgccaggaaattggcggccctcgctcttacactatcgcggtcgccagtctgggcaagggagtcgcttgtaaccccgcctgcttcataactcagttgctgcccgtgaaacggaagctgggcttctatgaatggactagcagactccgcagtcatattaatccgactggtacggtgctgctgcaactggagaataccatgcagatgtcacttaaggatcttctgtgagaacccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 3 - DNA, 11298 bp, synthetic construct
  misc_feature 1..11298: note = Dual promoter lentiviral vector comprising AGA transgene
  source 1..11298: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggttagaacagtctcttttcgtatgagtgcagtgagtggacgccgccttcgacctgggctgaagaagttcttttctcgaacttcagttcgccggaatacatcattgcccgcacctgtgtcaggctcttagcgccgatatcctggcatgaatgctgaattccggcgatcaggtaaggcacgaatttgtgaatactgcccttatcctggacagctccagacacgccctgtgcgactttgatcttgtctgcctcggaaaaatacctgttctgagaggacagatgcttatccatggcgtccagtgaccccatgcccctatatttcttcagtctgaacccatcactaaagaagtactcgccgggggcttctgtggttgcagccagcaggctgcccatcatcactgtgcttgcccccagagccagggcttttgcgatgtggcccacattctgaattcccccgtcagcgatcactgggactccgaatctccgggcatactcgtacaccttgtagacagcagttgcctgaggtcgtccacaggccagcacttcctgaatgatgcagattgatccactccccattccgaccctcagagcatccactcctgcgtcaatcaggtttttggcctgggctgcggtcacgacattgcctccgatgacctgcagatttgggtacttgtccttaatgtacttgatcatattaatctggaagatgctgtttccctggcttgaatccagcacgaccacgtccacccctgcctgagccagcagatccaggcgatatttatcgtcctcgtgtgtgccaatagcggctccacacagcagctgtttctttgcgtccttactagccagagggtaatctcgatttttcttcaggtcggtgcgggcaatgattgccaccagctcatcgtcttcattcacgataggcagttttcctttcttagaccgctgcagaatctcgttggcttccttcagtgtgatgccggcaggtgcgaccaccagatcttcgcgtttggtcataatctcttccagaaaacagtcatgctcttcctccttcaggaaatcgatgtctcgactagaaatgattcccaccagtcggctgcccattcgtccagtatctgtaatggggatgccgcaaaatccgtgcctagctttggcctcgaacacatcgcggaccctgtccttggggctcaggaccactgggtcggtgataaagccctgttcgtatttcttcacctttctgacctcattggcctgaaattctggagtgcagttatggtgaatgaacccgatcccgcctgtcagtgccatagcaatggccatgccagcctcggtgacagtgtccataggggagctcaccaggggtgtcttcagggtgattttcttggtcagggcagaagtcagatccacctggtctgcggtaaaatcaatatagccgggcaggatcaggaagtcgttgtaagtcagcccgtctccacaattaaacagctgctgggcggtcagtccatcatcagggacataggaagtgcctccagaaatcaggtagtcggccatggtggcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgccaccatgcaacttcgaaacccagagctccacctcggatgtgcccttgctctgaggttcctggcgctggtgtcttgggatatacccggagcacgcgctctggacaacgggctggcccggactccaaccatgggttggctccattgggaaaggtttatgtgcaacttggactgccaggaagaacccgactcctgtatttccgagaaactcttcatggagatggccgagctgatggttagcgaaggctggaaggatgccggttatgaatacttgtgtatcgacgattgttggatggctccccagcgggacagtgaaggacgactccaggcagatccgcaacggttccctcatggcatacggcagctcgccaattacgtgcacagcaagggtttgaagctggggatatatgctgacgtgggcaacaaaacctgtgctggtttccccggcagcttcggctactatgatatagatgcacaaaccttcgctgattggggcgtggacctgcttaaatttgacggctgttactgcgacagcttggaaaacctcgccgatggatataaacacatgagccttgcactcaatcggactggccggagcattgtctactcttgcgagtggccattgtacatgtggcctttccagaagcctaactatacggagattagacagtattgtaatcactggagaaactttgcagatatcgacgactcatggaagtccatcaaatctattctggactggacttcattcaatcaggagcgcatcgtcgatgttgccggtccaggtggatggaacgaccctgacatgctcgtaattgggaatttcggactgtcctggaatcagcaggtcacacagatggctttgtgggctatcatggcagccccactctttatgtctaacgatttgcggcatatttcaccacaggccaaagccctgctgcaagataaggacgtcatagcgattaaccaggacccactgggaaagcagggctaccagctgagacagggcgacaattttgaggtctgggaaagacctcttagcgggctggcgtgggccgtagccatgattaatcgccaggaaattggcggccctcgctcttacactatcgcggtcgccagtctgggcaagggagtcgcttgtaaccccgcctgcttcataactcagttgctgcccgtgaaacggaagctgggcttctatgaatggactagcagactccgcagtcatattaatccgactggtacggtgctgctgcaactggagaataccatgcagatgtcacttaaggatcttctgtgagaacccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 4 - DNA, 9095 bp, synthetic construct
  misc_feature 1..9095: note = Lentiviral vector comprising GBA transgene
  source 1..9095: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgctagcgccaccatggagttctcaagcccctctcgggaagaatgcccaaaacctctgtcacgggtgtctatcatggctggatcactgactggcctgctgctgctgcaggccgtgagctgggcctccggagcccggccttgcatcccaaagtctttcggctacagctccgtggtgtgcgtgtgcaacgccacctattgtgactccttcgatccccctacctttcccgccctgggcacattttctcggtacgagtctacacgcagcggcaggagaatggagctgagcatgggccctatccaggccaatcacaccggaacaggcctgctgctgaccctgcagccagagcagaagttccagaaggtgaagggctttggaggagcaatgacagacgcagccgccctgaacatcctggccctgtccccacccgcccagaatctgctgctgaagtcctacttctctgaggagggcatcggctataacatcatcagggtgcccatggccagctgcgacttttccatcagaacctacacatatgccgatacccctgacgatttccagctgcacaatttttccctgccagaggaggatacaaagctgaagatcccactgatccacagggccctgcagctggcccagaggcccgtgagcctgctggccagcccctggacctcccctacatggctgaagaccaacggcgccgtgaatggcaagggctctctgaagggacagccaggcgacatctaccaccagacatgggcccgctatttcgtgaagtttctggatgcctacgccgagcacaagctgcagttctgggccgtgaccgcagagaacgagccttctgccggcctgctgagcggctatcccttccagtgcctgggctttacacctgagcaccagagggactttatcgccagagatctgggcccaaccctggccaactccacacaccacaatgtgcggctgctgatgctggacgatcagcgcctgctgctgcctcactgggccaaggtggtgctgaccgacccagaggccgccaagtacgtgcacggcatcgccgtgcactggtatctggatttcctggcaccagcaaaggccaccctgggagagacacacaggctgttccctaacaccatgctgtttgccagcgaggcctgcgtgggctccaagttttgggagcagtccgtgcggctgggctcttgggacaggggcatgcagtactcccactctatcatcaccaatctgctgtatcacgtggtgggctggacagactggaacctggccctgaatccagagggcggccccaactgggtgagaaatttcgtggatagccccatcatcgtggacatcaccaaggatacattctacaagcagccaatgttttatcacctgggccacttctctaagtttatcccagagggcagccagagggtgggcctggtggccagccagaagaacgacctggatgcagtggccctgatgcaccctgacggctccgccgtggtggtggtgctgaatcgctctagcaaggacgtgcctctgaccatcaaggaccccgccgtgggctttctggagaccatttcacccggctattctattcatacctatctgtggaggaggcagtaacctgcaggggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 5 - DNA, 8681 bp, synthetic construct
  misc_feature 1..8681: note = Lentiviral vector comprising ASAH1 transgene
  source 1..8681: mol_type = other DNA
gggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacgccaccatgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgagcggccgcctcgaggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 6 - DNA, 10336 bp, synthetic construct
  misc_feature 1..10336: note = Lentiviral vector comprising GAA transgene
  source 1..10336: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgccaccatgggcgtgaggcaccccccttgctctcacaggctgctggccgtgtgcgcactggtgagcctggccaccgccgccctgctgggccacatcctgctgcacgacttcctgctggtgcccagggagctgtccggcagctccccagtgctggaggagacccacccagcacaccagcagggcgcctctcggccaggcccccgcgatgcacaggcacacccaggccggccccgcgccgtgccaacccagtgcgacgtgccacccaacagccggtttgactgtgcccccgataaggccatcacacaggagcagtgcgaggccaggggctgctgttatatccctgcaaagcagggcctccagggcgcccagatgggacagccatggtgtttctttcctccatcttaccccagctataagctggagaatctgtctagctccgagatgggctacacagccaccctgacaagaaccacaccaacattctttcccaaggacatcctgaccctgcggctggacgtgatgatggagacagagaaccgcctgcacttcaccatcaaggaccccgccaataggagatatgaggtgcctctggagaccccacacgtgcactctcgggcccctagcccactgtactccgtggagttctctgaggagccatttggcgtgatcgtgcggcgccagctggatggacgcgtgctgctgaacaccacagtggcccccctgttctttgccgaccagttcctccagctgagcacatccctgccctcccagtatatcaccggcctggccgagcacctgtctcctctgatgctgtctaccagctggacaaggatcaccctgtggaacagagacctggcaccaacccctggcgcaaatctgtacggcagccaccctttctatctggccctggaggatggaggctccgcccacggcgtgtttctgctgaactctaatgccatggacgtggtgctccagccaagccccgccctgtcctggcggtctaccggcggcatcctggacgtgtacatcttcctgggccctgagccaaagtccgtggtgcagcagtacctggacgtggtgggctatcctttcatgcccccttactggggactgggatttcacctgtgccgctggggctattctagcacagccatcacccggcaggtggtggagaacatgacccgcgcccactttccactggatgtgcagtggaatgacctggattacatggactccaggagagacttcaccttcaacaaggacggcttcagggattttcccgccatggtgcaggagctgcaccagggcggccggcgctacatgatgatcgtggaccccgccatctcctctagcggacctgccggcagctacagaccatatgacgagggcctgaggagaggcgtgttcatcacaaacgagaccggccagcctctgatcggcaaggtctggccaggctccaccgccttcccagacttcaccaatccaaccgccctggcctggtgggaggacatggtggccgagttccacgaccaggtgccttttgatggcatgtggatcgacatgaacgagccatctaatttcatcaggggcagcgaggacggctgccccaacaatgagctggagaacccaccatatgtgcctggcgtggtgggaggcaccctccaggcagcaaccatctgtgcctcctctcaccagtttctgtctacacactataacctgcacaatctgtacggactgaccgaggcaatcgccagccacagagccctggtgaaggccaggggcacaagacctttcgtgatctccaggtctacctttgccggacacggcagatacgcaggacactggaccggcgacgtgtggagcagctgggagcagctggcctctagcgtgccagagatcctccagttcaacctgctgggcgtgcccctggtgggagcagacgtgtgcggctttctgggcaatacatccgaggagctgtgcgtgaggtggacccagctgggagccttctatcccttcatgcgcaaccacaatagcctgctgtccctgcctcaggagccatacagcttctccgagcctgcacagcaggcaatgaggaaggccctgacactgcgctatgccctgctgccacacctgtacaccctgtttcaccaggcacacgtggcaggagagacagtggcccggcccctgttcctggagtttcctaaggattcctctacctggacagtggaccaccagctgctgtggggagaggccctgctgatcacccccgtgctccaggcaggcaaggcagaggtgacaggctatttccctctgggcacatggtacgacctccagaccgtgccagtggaggccctgggcagcctgcctccaccacctgccgccccccgcgagcctgccatccactccgagggacagtgggtgacactgccagcacctctggacaccatcaacgtgcacctgagggccggctatatcatccccctccagggccctggcctgaccacaaccgagtccagacagcagccaatggccctggccgtggccctgaccaagggaggcgaggccaggggcgagctgttctgggacgatggcgagtctctggaggtgctggagagaggcgcctacacacaggtcatcttcctggccaggaacaatacaatcgtgaatgagctggtgagagtgacctctgagggagcaggactccagctccagaaggtgacagtgctgggagtggcaaccgcaccacagcaggtgctgagcaacggcgtgcccgtgagcaatttcacatactcccctgataccaaggtgctggacatctgcgtgagcctgctgatgggcgagcagtttctggtgtcctggtgttgagaacccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 7 - DNA, 1611 bp, synthetic construct
  misc_feature 1..1611: note = GBA transgene
  source 1..1611: mol_type = other DNA
atggagttctcaagcccctctcgggaagaatgcccaaaacctctgtcacgggtgtctatcatggctggatcactgactggcctgctgctgctgcaggccgtgagctgggcctccggagcccggccttgcatcccaaagtctttcggctacagctccgtggtgtgcgtgtgcaacgccacctattgtgactccttcgatccccctacctttcccgccctgggcacattttctcggtacgagtctacacgcagcggcaggagaatggagctgagcatgggccctatccaggccaatcacaccggaacaggcctgctgctgaccctgcagccagagcagaagttccagaaggtgaagggctttggaggagcaatgacagacgcagccgccctgaacatcctggccctgtccccacccgcccagaatctgctgctgaagtcctacttctctgaggagggcatcggctataacatcatcagggtgcccatggccagctgcgacttttccatcagaacctacacatatgccgatacccctgacgatttccagctgcacaatttttccctgccagaggaggatacaaagctgaagatcccactgatccacagggccctgcagctggcccagaggcccgtgagcctgctggccagcccctggacctcccctacatggctgaagaccaacggcgccgtgaatggcaagggctctctgaagggacagccaggcgacatctaccaccagacatgggcccgctatttcgtgaagtttctggatgcctacgccgagcacaagctgcagttctgggccgtgaccgcagagaacgagccttctgccggcctgctgagcggctatcccttccagtgcctgggctttacacctgagcaccagagggactttatcgccagagatctgggcccaaccctggccaactccacacaccacaatgtgcggctgctgatgctggacgatcagcgcctgctgctgcctcactgggccaaggtggtgctgaccgacccagaggccgccaagtacgtgcacggcatcgccgtgcactggtatctggatttcctggcaccagcaaaggccaccctgggagagacacacaggctgttccctaacaccatgctgtttgccagcgaggcctgcgtgggctccaagttttgggagcagtccgtgcggctgggctcttgggacaggggcatgcagtactcccactctatcatcaccaatctgctgtatcacgtggtgggctggacagactggaacctggccctgaatccagagggcggccccaactgggtgagaaatttcgtggatagccccatcatcgtggacatcaccaaggatacattctacaagcagccaatgttttatcacctgggccacttctctaagtttatcccagagggcagccagagggtgggcctggtggccagccagaagaacgacctggatgcagtggccctgatgcaccctgacggctccgccgtggtggtggtgctgaatcgctctagcaaggacgtgcctctgaccatcaaggaccccgccgtgggctttctggagaccatttcacccggctattctattcatacctatctgtggaggaggcagtaa

SEQ ID NO: 8 - DNA, 1188 bp, synthetic construct
  misc_feature 1..1188: note = ASAH1 transgene
  source 1..1188: mol_type = other DNA
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 9 - DNA, 2859 bp, synthetic construct
  misc_feature 1..2859: note = GAA transgene
  source 1..2859: mol_type = other DNA
atgggcgtgaggcaccccccttgctctcacaggctgctggccgtgtgcgcactggtgagcctggccaccgccgccctgctgggccacatcctgctgcacgacttcctgctggtgcccagggagctgtccggcagctccccagtgctggaggagacccacccagcacaccagcagggcgcctctcggccaggcccccgcgatgcacaggcacacccaggccggccccgcgccgtgccaacccagtgcgacgtgccacccaacagccggtttgactgtgcccccgataaggccatcacacaggagcagtgcgaggccaggggctgctgttatatccctgcaaagcagggcctccagggcgcccagatgggacagccatggtgtttctttcctccatcttaccccagctataagctggagaatctgtctagctccgagatgggctacacagccaccctgacaagaaccacaccaacattctttcccaaggacatcctgaccctgcggctggacgtgatgatggagacagagaaccgcctgcacttcaccatcaaggaccccgccaataggagatatgaggtgcctctggagaccccacacgtgcactctcgggcccctagcccactgtactccgtggagttctctgaggagccatttggcgtgatcgtgcggcgccagctggatggacgcgtgctgctgaacaccacagtggcccccctgttctttgccgaccagttcctccagctgagcacatccctgccctcccagtatatcaccggcctggccgagcacctgtctcctctgatgctgtctaccagctggacaaggatcaccctgtggaacagagacctggcaccaacccctggcgcaaatctgtacggcagccaccctttctatctggccctggaggatggaggctccgcccacggcgtgtttctgctgaactctaatgccatggacgtggtgctccagccaagccccgccctgtcctggcggtctaccggcggcatcctggacgtgtacatcttcctgggccctgagccaaagtccgtggtgcagcagtacctggacgtggtgggctatcctttcatgcccccttactggggactgggatttcacctgtgccgctggggctattctagcacagccatcacccggcaggtggtggagaacatgacccgcgcccactttccactggatgtgcagtggaatgacctggattacatggactccaggagagacttcaccttcaacaaggacggcttcagggattttcccgccatggtgcaggagctgcaccagggcggccggcgctacatgatgatcgtggaccccgccatctcctctagcggacctgccggcagctacagaccatatgacgagggcctgaggagaggcgtgttcatcacaaacgagaccggccagcctctgatcggcaaggtctggccaggctccaccgccttcccagacttcaccaatccaaccgccctggcctggtgggaggacatggtggccgagttccacgaccaggtgccttttgatggcatgtggatcgacatgaacgagccatctaatttcatcaggggcagcgaggacggctgccccaacaatgagctggagaacccaccatatgtgcctggcgtggtgggaggcaccctccaggcagcaaccatctgtgcctcctctcaccagtttctgtctacacactataacctgcacaatctgtacggactgaccgaggcaatcgccagccacagagccctggtgaaggccaggggcacaagacctttcgtgatctccaggtctacctttgccggacacggcagatacgcaggacactggaccggcgacgtgtggagcagctgggagcagctggcctctagcgtgccagagatcctccagttcaacctgctgggcgtgcccctggtgggagcagacgtgtgcggctttctgggcaatacatccgaggagctgtgcgtgaggtggacccagctgggagccttctatcccttcatgcgcaaccacaatagcctgctgtccctgcctcaggagccatacagcttctccgagcctgcacagcaggcaatgaggaaggccctgacactgcgctatgccctgctgccacacctgtacaccctgtttcaccaggcacacgtggcaggagagacagtggcccggcccctgttcctggagtttcctaaggattcctctacctggacagtggaccaccagctgctgtggggagaggccctgctgatcacccccgtgctccaggcaggcaaggcagaggtgacaggctatttccctctgggcacatggtacgacctccagaccgtgccagtggaggccctgggcagcctgcctccaccacctgccgccccccgcgagcctgccatccactccgagggacagtgggtgacactgccagcacctctggacaccatcaacgtgcacctgagggccggctatatcatccccctccagggccctggcctgaccacaaccgagtccagacagcagccaatggccctggccgtggccctgaccaagggaggcgaggccaggggcgagctgttctgggacgatggcgagtctctggaggtgctggagagaggcgcctacacacaggtcatcttcctggccaggaacaatacaatcgtgaatgagctggtgagagtgacctctgagggagcaggactccagctccagaaggtgacagtgctgggagtggcaaccgcaccacagcaggtgctgagcaacggcgtgcccgtgagcaatttcacatactcccctgataccaaggtgctggacatctgcgtgagcctgctgatgggcgagcagtttctggtgtcctggtgttga

SEQ ID NO: 10 - DNA, 8523 bp, synthetic construct
  misc_feature 1..8523: note = Dual promoter lentiviral vector
  source 1..8523: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggcggatggcgttaacatatgacaactttctcccgggtaatctgaccgttcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcgggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 11 - DNA, 10025 bp, synthetic construct
  misc_feature 1..10025: note = Dual promoter lentiviral vector comprising IMPDH2(IY) resistance gene
  source 1..10025: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggttagaacagtctcttttcgtatgagtgcagtgagtggacgccgccttcgacctgggctgaagaagttcttttctcgaacttcagttcgccggaatacatcattgcccgcacctgtgtcaggctcttagcgccgatatcctggcatgaatgctgaattccggcgatcaggtaaggcacgaatttgtgaatactgcccttatcctggacagctccagacacgccctgtgcgactttgatcttgtctgcctcggaaaaatacctgttctgagaggacagatgcttatccatggcgtccagtgaccccatgcccctatatttcttcagtctgaacccatcactaaagaagtactcgccgggggcttctgtggttgcagccagcaggctgcccatcatcactgtgcttgcccccagagccagggcttttgcgatgtggcccacattctgaattcccccgtcagcgatcactgggactccgaatctccgggcatactcgtacaccttgtagacagcagttgcctgaggtcgtccacaggccagcacttcctgaatgatgcagattgatccactccccattccgaccctcagagcatccactcctgcgtcaatcaggtttttggcctgggctgcggtcacgacattgcctccgatgacctgcagatttgggtacttgtccttaatgtacttgatcatattaatctggaagatgctgtttccctggcttgaatccagcacgaccacgtccacccctgcctgagccagcagatccaggcgatatttatcgtcctcgtgtgtgccaatagcggctccacacagcagctgtttctttgcgtccttactagccagagggtaatctcgatttttcttcaggtcggtgcgggcaatgattgccaccagctcatcgtcttcattcacgataggcagttttcctttcttagaccgctgcagaatctcgttggcttccttcagtgtgatgccggcaggtgcgaccaccagatcttcgcgtttggtcataatctcttccagaaaacagtcatgctcttcctccttcaggaaatcgatgtctcgactagaaatgattcccaccagtcggctgcccattcgtccagtatctgtaatggggatgccgcaaaatccgtgcctagctttggcctcgaacacatcgcggaccctgtccttggggctcaggaccactgggtcggtgataaagccctgttcgtatttcttcacctttctgacctcattggcctgaaattctggagtgcagttatggtgaatgaacccgatcccgcctgtcagtgccatagcaatggccatgccagcctcggtgacagtgtccataggggagctcaccaggggtgtcttcagggtgattttcttggtcagggcagaagtcagatccacctggtctgcggtaaaatcaatatagccgggcaggatcaggaagtcgttgtaagtcagcccgtctccacaattaaacagctgctgggcggtcagtccatcatcagggacataggaagtgcctccagaaatcaggtagtcggccatggtggcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcgggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg